SEQUENCE LISTING (236 sequences)

SEQ ID NO: 1 - RNA, 80 nt, synthetic construct
  misc_feature 1..80: note = Synthetic aptamer sequences
  source 1..80: mol_type = other RNA
gggtgcatcgtttacgcgattagtctgataaacaaaaagatttcgctaaaaatcaatctgctgctgaggaaggatatgag

SEQ ID NO: 2 - RNA, 80 nt, synthetic construct
  misc_feature 1..80: note = Synthetic aptamer sequences
  source 1..80: mol_type = other RNA
gggtgcatcgtttacgcagatagcagcaggaatcaagcggtaggagtctagcagaagctgctgctgaggaaggatatgag

SEQ ID NO: 3 - RNA, 80 nt, synthetic construct
  misc_feature 1..80: note = Synthetic aptamer sequences
  source 1..80: mol_type = other RNA
gggtgcatcgtttacgcattttcgttttatttcagtttaattgcgtttagtatctggctgctgctgaggaaggatatgag

SEQ ID NO: 4 - RNA, 80 nt, synthetic construct
  misc_feature 1..80: note = Synthetic aptamer sequences
  source 1..80: mol_type = other RNA
gggtgcatcgtttacgcgcaacataaaaatttaaagtgctcagttgtcaatctatgactgctgctgaggaaggatatgag

SEQ ID NO: 5 - RNA, 80 nt, synthetic construct
  misc_feature 1..80: note = Synthetic aptamer sequences
  source 1..80: mol_type = other RNA
gggtgcatcgtttacgcgtaaatggtccgctattaaaagaaaagaatgaagtctcagctgctgctgaggaaggatatgag

SEQ ID NO: 6 - RNA, 80 nt, synthetic construct
  misc_feature 1..80: note = Synthetic aptamer sequences
  source 1..80: mol_type = other RNA
gggtgcatcgtttacgctattttcatttgtttttttaatttactagtgtaaacaatcctgctgctgaggaaggatatgag

SEQ ID NO: 7 - RNA, 80 nt, synthetic construct
  misc_feature 1..80: note = Synthetic aptamer sequences
  source 1..80: mol_type = other RNA
gggtgcatcgtttacgcgtaaataagtagataaagtggcagtttgttttccttggaactgctgctgaggaaggatatgag

SEQ ID NO: 8 - RNA, 80 nt, synthetic construct
  misc_feature 1..80: note = Synthetic aptamer sequences
  source 1..80: mol_type = other RNA
gggtgcatcgtttacgcgtaaaaattaaagagattaaggtccttaagcagttttgtcctgctgctgaggaaggatatgag

SEQ ID NO: 9 - RNA, 80 nt, synthetic construct
  misc_feature 1..80: note = Synthetic aptamer sequences
  source 1..80: mol_type = other RNA
gggtgcatcgtttacgcgtaaaaaaatcaaaacttcagcaaattatttatcaacgtcctgctgctgaggaaggatatgag

SEQ ID NO: 10 - RNA, 80 nt, synthetic construct
  misc_feature 1..80: note = Synthetic aptamer sequences
  source 1..80: mol_type = other RNA
gggtgcatcgtttacgcgtaaaataaattaaaaagaacttcttcagcaatcaatatcctgctgctgaggaaggatatgag

SEQ ID NO: 11 - RNA, 80 nt, synthetic construct
  misc_feature 1..80: note = Synthetic aptamer sequences
  source 1..80: mol_type = other RNA
gggtgcatcgtttacgcgtaaataaaaatgaaaaattgtctctcagctttcaaagtcctgctgctgaggaaggatatgag

SEQ ID NO: 12 - RNA, 80 nt, synthetic construct
  misc_feature 1..80: note = Synthetic aptamer sequences
  source 1..80: mol_type = other RNA
gggtgcatcgtttacgcgtaaaaaaaaaatatcttcggagaattcagcaattttatcctgctgctgaggaaggatatgag

SEQ ID NO: 13 - RNA, 80 nt, synthetic construct
  misc_feature 1..80: note = Synthetic aptamer sequences
  source 1..80: mol_type = other RNA
gggtgcatcgtttacgcgtaaaaattttcatctcagcaattaaatccaaagaatccactgctgctgaggaaggatatgag

SEQ ID NO: 14 - RNA, 80 nt, synthetic construct
  misc_feature 1..80: note = Synthetic aptamer sequences
  source 1..80: mol_type = other RNA
gggtgcatcgtttacgcgtaaaatatatcagcaaagtagtttaagcctcctcagtttctgctgctgaggaaggatatgag

SEQ ID NO: 15 - RNA, 80 nt, synthetic construct
  misc_feature 1..80: note = Synthetic aptamer sequences
  source 1..80: mol_type = other RNA
gggtgcatcgtttacgcgtaaattatgaaaaatacagcaaggatttaacctcagtttctgctgctgaggaaggatatgag

SEQ ID NO: 16 - RNA, 80 nt, synthetic construct
  misc_feature 1..80: note = Synthetic aptamer sequences
  source 1..80: mol_type = other RNA
gggtgcatcgtttacgcgtaaaataaataaatcttcaaagtacagacctcgatttttctgctgctgaggaaggatatgag

SEQ ID NO: 17 - RNA, 80 nt, synthetic construct
  misc_feature 1..80: note = Synthetic aptamer sequences
  source 1..80: mol_type = other RNA
gggtgcatcgtttacgcttataggtattagacattttcaattaaagtgaattagtgtctgctgctgaggaaggatatgag

SEQ ID NO: 18 - RNA, 80 nt, synthetic construct
  misc_feature 1..80: note = Synthetic aptamer sequences
  source 1..80: mol_type = other RNA
gggtgcatcgtttacgcgtaaaatgtgacagcaggataataaaataagtactcagtactgctgctgaggaaggatatgag

SEQ ID NO: 19 - RNA, 80 nt, synthetic construct
  misc_feature 1..80: note = Synthetic aptamer sequences
  source 1..80: mol_type = other RNA
gggtgcatcgtttacgcgtaattaagaaaaataaaagtactctgcagtttttatccactgctgctgaggaaggatatgag

SEQ ID NO: 20 - RNA, 80 nt, synthetic construct
  misc_feature 1..80: note = Synthetic aptamer sequences
  source 1..80: mol_type = other RNA
gggtgcatcgtttacgcgtaaaaataaaattttcccagaccagttatctgccttaaactgctgctgaggaaggatatgag

SEQ ID NO: 21 - RNA, 80 nt, synthetic construct
  misc_feature 1..80: note = Synthetic aptamer sequences
  source 1..80: mol_type = other RNA
gggtgcatcgtttacgcgtaaagaaaaaaatcagcttttagtcgccttccattttgactgctgctgaggaaggatatgag

SEQ ID NO: 22 - RNA, 80 nt, synthetic construct
  misc_feature 1..80: note = Synthetic aptamer sequences
  source 1..80: mol_type = other RNA
gggtgcatcgtttacgcgtaaataaataatcaaaattacactcagtggcaatttcctctgctgctgaggaaggatatgag

SEQ ID NO: 23 - RNA, 80 nt, synthetic construct
  misc_feature 1..80: note = Synthetic aptamer sequences
  source 1..80: mol_type = other RNA
gggtgcatcgtttacgcgtaaaatacaggatacgacaataactcagcagattttatcctgctgctgaggaaggatatgag

SEQ ID NO: 24 - RNA, 80 nt, synthetic construct
  misc_feature 1..80: note = Synthetic aptamer sequences
  source 1..80: mol_type = other RNA
gggtgcatcgtttacgcgttaaaaattgtgcactgagatgacgcagcattaactacactgctgctgaggaaggatatgag

SEQ ID NO: 25 - RNA, 80 nt, synthetic construct
  misc_feature 1..80: note = Synthetic aptamer sequences
  source 1..80: mol_type = other RNA
gggtgcatcgtttacgcgtaaataaaaattaatcagcaattttccactcagttgtacctgctgctgaggaaggatatgag

SEQ ID NO: 26 - RNA, 80 nt, synthetic construct
  misc_feature 1..80: note = Synthetic aptamer sequences
  source 1..80: mol_type = other RNA
gggtgcatcgtttacgcgtaaaaataaaaaatctcgatcactgcagttttattccggctgctgctgaggaaggatatgag

SEQ ID NO: 27 - RNA, 80 nt, synthetic construct
  misc_feature 1..80: note = Synthetic aptamer sequences
  source 1..80: mol_type = other RNA
gggtgcatcgtttacgcgtaaacaaatatcgattaaaataaaatctcagcaagaatcctgctgctgaggaaggatatgag

SEQ ID NO: 28 - RNA, 80 nt, synthetic construct
  misc_feature 1..80: note = Synthetic aptamer sequences
  source 1..80: mol_type = other RNA
gggtgcatcgtttacgcgtaaaataaataaaattatcccaggagcaaattttcttcgctgctgctgaggaaggatatgag

SEQ ID NO: 29 - RNA, 80 nt, synthetic construct
  misc_feature 1..80: note = Synthetic aptamer sequences
  source 1..80: mol_type = other RNA
gggtgcatcgtttacgcgtagaagaattaatagtggacatatcaatagcagtttatcctgctgctgaggaaggatatgag

SEQ ID NO: 30 - RNA, 80 nt, synthetic construct
  misc_feature 1..80: note = Synthetic aptamer sequences
  source 1..80: mol_type = other RNA
gggtgcatcgtttacgcgtaaacatattcagcagttaaaatttagtaggttcagtagctgctgctgaggaaggatatgag

SEQ ID NO: 31 - RNA, 80 nt, synthetic construct
  misc_feature 1..80: note = Synthetic aptamer sequences
  source 1..80: mol_type = other RNA
gggtgcatcgtttacgcgtaaaaaagataaaacttagttgcagaatttgccttcattctgctgctgaggaaggatatgag

SEQ ID NO: 32 - RNA, 80 nt, synthetic construct
  misc_feature 1..80: note = Synthetic aptamer sequences
  source 1..80: mol_type = other RNA
gggtgcatcgtttacgcgtaaaaagtttgatggaagcagattagtttagtcaaatttctgctgctgaggaaggatatgag

SEQ ID NO: 33 - RNA, 80 nt, synthetic construct
  misc_feature 1..80: note = Synthetic aptamer sequences
  source 1..80: mol_type = other RNA
gggtgcatcgtttacgcgtaaaatgaaataaggaatccttcagcagtatttatccttctgctgctgaggaaggatatgag

SEQ ID NO: 34 - RNA, 80 nt, synthetic construct
  misc_feature 1..80: note = Synthetic aptamer sequences
  source 1..80: mol_type = other RNA
gggtgcatcgtttacgcgtaaagaataaaaatgacaaaattctcagcttttgtcaacctgctgctgaggaaggatatgag

SEQ ID NO: 35 - RNA, 80 nt, synthetic construct
  misc_feature 1..80: note = Synthetic aptamer sequences
  source 1..80: mol_type = other RNA
gggtgcatcgtttacgcgtaaaaaatgaaatgaaaaaattctcagctgtctatcttcctgctgctgaggaaggatatgag

SEQ ID NO: 36 - RNA, 80 nt, synthetic construct
  misc_feature 1..80: note = Synthetic aptamer sequences
  source 1..80: mol_type = other RNA
gggtgcatcgtttacgcgtaaataagtaaaaaactcagttttcagttaagtatccaactgctgctgaggaaggatatgag

SEQ ID NO: 37 - RNA, 80 nt, synthetic construct
  misc_feature 1..80: note = Synthetic aptamer sequences
  source 1..80: mol_type = other RNA
gggtgcatcgtttacgcgtaaatttcagcagagtaataataacacttcttcagtttgctgctgctgaggaaggatatgag

SEQ ID NO: 38 - RNA, 80 nt, synthetic construct
  misc_feature 1..80: note = Synthetic aptamer sequences
  source 1..80: mol_type = other RNA
gggtgcatcgtttacgcgtaaaattaagaagtattatcagttagctttttcttccaactgctgctgaggaaggatatgag

SEQ ID NO: 39 - RNA, 80 nt, synthetic construct
  misc_feature 1..80: note = Synthetic aptamer sequences
  source 1..80: mol_type = other RNA
gggtgcatcgtttacgcgtaaaataaaaagttttcctatcagcaaactcacaaattcctgctgctgaggaaggatatgag

SEQ ID NO: 40 - RNA, 80 nt, synthetic construct
  misc_feature 1..80: note = Synthetic aptamer sequences
  source 1..80: mol_type = other RNA
gggtgcatcgtttacgcgtaaaatgaaatgtaaaagaattgaacttggcagattttcctgctgctgaggaaggatatgag

SEQ ID NO: 41 - RNA, 80 nt, synthetic construct
  misc_feature 1..80: note = Synthetic aptamer sequences
  source 1..80: mol_type = other RNA
gggtgcatcgtttacgcgtaaattaaagtagcagtaatttcagcagtttttacctctctgctgctgaggaaggatatgag

SEQ ID NO: 42 - RNA, 80 nt, synthetic construct
  misc_feature 1..80: note = Synthetic aptamer sequences
  source 1..80: mol_type = other RNA
gggtgcatcgtttacgcgtaaataaaggataaaataatttcagggcagtttctcatcctgctgctgaggaaggatatgag

SEQ ID NO: 43 - RNA, 80 nt, synthetic construct
  misc_feature 1..80: note = Synthetic aptamer sequences
  source 1..80: mol_type = other RNA
gggtgcatcgtttacgcaggatcgttttaagtaaaataaaagatttccttggtaatcctgctgctgaggaaggatatgag

SEQ ID NO: 44 - RNA, 80 nt, synthetic construct
  misc_feature 1..80: note = Synthetic aptamer sequences
  source 1..80: mol_type = other RNA
gggtgcatcgtttacgcgtaaaataaagatcaattaaaggctttgatcgattttcctctgctgctgaggaaggatatgag

SEQ ID NO: 45 - RNA, 80 nt, synthetic construct
  misc_feature 1..80: note = Synthetic aptamer sequences
  source 1..80: mol_type = other RNA
gggtgcatcgtttacgcgtaaaaattagagattaaaatagttcctttcagttttgtcctgctgctgaggaaggatatgag

SEQ ID NO: 46 - RNA, 80 nt, synthetic construct
  misc_feature 1..80: note = Synthetic aptamer sequences
  source 1..80: mol_type = other RNA
gggtgcatcgtttacgcgtaaaattgacaatgtgaaaagcagacagcaaatattcctctgctgctgaggaaggatatgag

SEQ ID NO: 47 - RNA, 80 nt, synthetic construct
  misc_feature 1..80: note = Synthetic aptamer sequences
  source 1..80: mol_type = other RNA
gggtgcatcgtttacgcgtaaataaccagttatacagaaagatctcagcaatttatcctgctgctgaggaaggatatgag

SEQ ID NO: 48 - RNA, 80 nt, synthetic construct
  misc_feature 1..80: note = Synthetic aptamer sequences
  source 1..80: mol_type = other RNA
gggtgcatcgtttacgcttacagaaggattgcaccacatgcgtactcgatgaaacacctgctgctgaggaaggatatgag

SEQ ID NO: 49 - RNA, 80 nt, synthetic construct
  misc_feature 1..80: note = Synthetic aptamer sequences
  source 1..80: mol_type = other RNA
gggtgcatcgtttacgcgtaaaataataattaaactcagcaaattcaatccaactttctgctgctgaggaaggatatgag

SEQ ID NO: 50 - RNA, 80 nt, synthetic construct
  misc_feature 1..80: note = Synthetic aptamer sequences
  source 1..80: mol_type = other RNA
gggtgcatcgtttacgcgtaaacaagaataaattcagcagtggttttgatcctttgactgctgctgaggaaggatatgag

SEQ ID NO: 51 - RNA, 80 nt, synthetic construct
  misc_feature 1..80: note = Synthetic aptamer sequences
  source 1..80: mol_type = other RNA
gggtgcatcgtttacgcgtaaattaatcagattgaacaaaagttttccctcagttttctgctgctgaggaaggatatgag

SEQ ID NO: 52 - RNA, 80 nt, synthetic construct
  misc_feature 1..80: note = Synthetic aptamer sequences
  source 1..80: mol_type = other RNA
gggtgcatcgtttacgcgtaaagaaaaacatcagagcagttataatagtcctttttcctgctgctgaggaaggatatgag

SEQ ID NO: 53 - RNA, 80 nt, synthetic construct
  misc_feature 1..80: note = Synthetic aptamer sequences
  source 1..80: mol_type = other RNA
gggtgcatcgtttacgcgtaaagaaaataaacttgatcaaacttagcagtttttatcctgctgctgaggaaggatatgag

SEQ ID NO: 54 - RNA, 80 nt, synthetic construct
  misc_feature 1..80: note = Synthetic aptamer sequences
  source 1..80: mol_type = other RNA
gggtgcatcgtttacgcattttcgttatatttctggtttttatgcgtgagaatcctgctgctgctgaggaaggatatgag

SEQ ID NO: 55 - RNA, 80 nt, synthetic construct
  misc_feature 1..80: note = Synthetic aptamer sequences
  source 1..80: mol_type = other RNA
gggtgcatcgtttacgcgtaaaaataagatctcacagcgacaaatttttcttccagtctgctgctgaggaaggatatgag

SEQ ID NO: 56 - RNA, 80 nt, synthetic construct
  misc_feature 1..80: note = Synthetic aptamer sequences
  source 1..80: mol_type = other RNA
gggtgcatcgtttacgcgtaaatttaagacatgacagcagacattttatcttcagacctgctgctgaggaaggatatgag

SEQ ID NO: 57 - RNA, 80 nt, synthetic construct
  misc_feature 1..80: note = Synthetic aptamer sequences
  source 1..80: mol_type = other RNA
gggtgcatcgtttacgcgtaataacagaaatataactcagctgaattaatttttccgctgctgctgaggaaggatatgag

SEQ ID NO: 58 - RNA, 80 nt, synthetic construct
  misc_feature 1..80: note = Synthetic aptamer sequences
  source 1..80: mol_type = other RNA
gggtgcatcgtttacgcgtaaaaataaattccaaaatattcagcagaaatcctcgaactgctgctgaggaaggatatgag

SEQ ID NO: 59 - RNA, 80 nt, synthetic construct
  misc_feature 1..80: note = Synthetic aptamer sequences
  source 1..80: mol_type = other RNA
gggtgcatcgtttacgcgtaaaaataataggttccaatcaagcagtacaaaattcctctgctgctgaggaaggatatgag

SEQ ID NO: 60 - RNA, 80 nt, synthetic construct
  misc_feature 1..80: note = Synthetic aptamer sequences
  source 1..80: mol_type = other RNA
gggtgcatcgtttacgcgtaaaaaatctaaaaagatatcagcaggcaaattttccttctgctgctgaggaaggatatgag

SEQ ID NO: 61 - RNA, 80 nt, synthetic construct
  misc_feature 1..80: note = Synthetic aptamer sequences
  source 1..80: mol_type = other RNA
gggtgcatcgtttacgcgtaaaataaagaggataactacaatcatcagcaatcatatctgctgctgaggaaggatatgag

SEQ ID NO: 62 - RNA, 80 nt, synthetic construct
  misc_feature 1..80: note = Synthetic aptamer sequences
  source 1..80: mol_type = other RNA
gggtgcatcgtttacgcgtaaatttagtagaaaggaaagacgaagtttcctcagtttctgctgctgaggaaggatatgag

SEQ ID NO: 63 - RNA, 80 nt, synthetic construct
  misc_feature 1..80: note = Synthetic aptamer sequences
  source 1..80: mol_type = other RNA
gggtgcatcgtttacgcgtaaaaataatagatctcagaatatgaaagcagttctttcctgctgctgaggaaggatatgag

SEQ ID NO: 64 - RNA, 80 nt, synthetic construct
  misc_feature 1..80: note = Synthetic aptamer sequences
  source 1..80: mol_type = other RNA
gggtgcatcgtttacgcgtaacaagatattcacagcagattttaaaaaattcctcgtctgctgctgaggaaggatatgag

SEQ ID NO: 65 - RNA, 80 nt, synthetic construct
  misc_feature 1..80: note = Synthetic aptamer sequences
  source 1..80: mol_type = other RNA
gggtgcatcgtttacgcgtaaaaagttgacaattaataaaatcttcttagcattttcctgctgctgaggaaggatatgag

SEQ ID NO: 66 - RNA, 80 nt, synthetic construct
  misc_feature 1..80: note = Synthetic aptamer sequences
  source 1..80: mol_type = other RNA
gggtgcatcgtttacgcgtaaaacaaaatgaaacttatagctcagcatattttgatcctgctgctgaggaaggatatgag

SEQ ID NO: 67 - RNA, 80 nt, synthetic construct
  misc_feature 1..80: note = Synthetic aptamer sequences
  source 1..80: mol_type = other RNA
gggtgcatcgtttacgcgtaaattatcaaaaaagcagatttaagtatacctcagttactgctgctgaggaaggatatgag

SEQ ID NO: 68 - RNA, 80 nt, synthetic construct
  misc_feature 1..80: note = Synthetic aptamer sequences
  source 1..80: mol_type = other RNA
gggtgcatcgtttacgcgtaaataaaatagctcagcaaggaagtttttttcctcaaactgctgctgaggaaggatatgag

SEQ ID NO: 69 - RNA, 80 nt, synthetic construct
  misc_feature 1..80: note = Synthetic aptamer sequences
  source 1..80: mol_type = other RNA
gggtgcatcgtttacgcgtaaatttgagaaaagaacagcagactcaaatctttttaactgctgctgaggaaggatatgag

SEQ ID NO: 70 - RNA, 80 nt, synthetic construct
  misc_feature 1..80: note = Synthetic aptamer sequences
  source 1..80: mol_type = other RNA
gggtgcatcgtttacgcgtaacagaaaattaagctcagcaatagtaattatcctagtctgctgctgaggaaggatatgag

SEQ ID NO: 71 - RNA, 80 nt, synthetic construct
  misc_feature 1..80: note = Synthetic aptamer sequences
  source 1..80: mol_type = other RNA
gggtgcatcgtttacgcgtaatgaaaataaatcagtctcacagcattttaaaacttcctgctgctgaggaaggatatgag

SEQ ID NO: 72 - RNA, 80 nt, synthetic construct
  misc_feature 1..80: note = Synthetic aptamer sequences
  source 1..80: mol_type = other RNA
gggtgcatcgtttacgcgtatttacaagcaacaaagttacaatcagcagaatttatcctgctgctgaggaaggatatgag

SEQ ID NO: 73 - RNA, 80 nt, synthetic construct
  misc_feature 1..80: note = Synthetic aptamer sequences
  source 1..80: mol_type = other RNA
gggtgcatcgtttacgcgtaaaaaattgtctatagcacttttagattcccaaactaactgctgctgaggaaggatatgag

SEQ ID NO: 74 - RNA, 80 nt, synthetic construct
  misc_feature 1..80: note = Synthetic aptamer sequences
  source 1..80: mol_type = other RNA
gggtgcatcgtttacgcgtaaaaaaatcagcaaaatcgaaaactcatgcagtttgtcctgctgctgaggaaggatatgag

SEQ ID NO: 75 - RNA, 80 nt, synthetic construct
  misc_feature 1..80: note = Synthetic aptamer sequences
  source 1..80: mol_type = other RNA
gggtgcatcgtttacgcgtaaaaaattccttaaaaatttaactaactggataggtctctgctgctgaggaaggatatgag

SEQ ID NO: 76 - RNA, 80 nt, synthetic construct
  misc_feature 1..80: note = Synthetic aptamer sequences
  source 1..80: mol_type = other RNA
gggtgcatcgtttacgcgtaaaacaaaatttctgacagcaattccttcgttaaaaatctgctgctgaggaaggatatgag

SEQ ID NO: 77 - RNA, 80 nt, synthetic construct
  misc_feature 1..80: note = Synthetic aptamer sequences
  source 1..80: mol_type = other RNA
gggtgcatcgtttacgcgtaaattattaaaaaaatcagcaaagtttatttcccacggctgctgctgaggaaggatatgag

SEQ ID NO: 78 - RNA, 80 nt, synthetic construct
  misc_feature 1..80: note = Synthetic aptamer sequences
  source 1..80: mol_type = other RNA
gggtgcatcgtttacgcgtaattaatcaaacaatagcagcaaatctcagcaattttcctgctgctgaggaaggatatgag

SEQ ID NO: 79 - RNA, 80 nt, synthetic construct
  misc_feature 1..80: note = Synthetic aptamer sequences
  source 1..80: mol_type = other RNA
gggtgcatcgtttacgcgtaatttgaaagtctcataaatttttttttttttttcaatctgctgctgaggaaggatatgag

SEQ ID NO: 80 - RNA, 80 nt, synthetic construct
  misc_feature 1..80: note = Synthetic aptamer sequences
  source 1..80: mol_type = other RNA
gggtgcatcgtttacgcgtaaaaattcagcatgatttcaattactcctttcattgatctgctgctgaggaaggatatgag

SEQ ID NO: 81 - RNA, 80 nt, synthetic construct
  misc_feature 1..80: note = Synthetic aptamer sequences
  source 1..80: mol_type = other RNA
gggtgcatcgtttacgcgtaaaataaataaaaatcagtagcaatctttctcacagtgctgctgctgaggaaggatatgag

SEQ ID NO: 82 - RNA, 80 nt, synthetic construct
  misc_feature 1..80: note = Synthetic aptamer sequences
  source 1..80: mol_type = other RNA
gggtgcatcgtttacgcgtaaataaaaagcagatctcagcaaaactcgtaaattcaactgctgctgaggaaggatatgag

SEQ ID NO: 83 - RNA, 80 nt, synthetic construct
  misc_feature 1..80: note = Synthetic aptamer sequences
  source 1..80: mol_type = other RNA
gggtgcatcgtttacgcgtaaataatgaaggactcagacagttaaaagatgcattaactgctgctgaggaaggatatgag

SEQ ID NO: 84 - RNA, 80 nt, synthetic construct
  misc_feature 1..80: note = Synthetic aptamer sequences
  source 1..80: mol_type = other RNA
gggtgcatcgtttacgcgtaaaaaagatcaatatgaaaatcagcagttaatatcttcctgctgctgaggaaggatatgag

SEQ ID NO: 85 - RNA, 80 nt, synthetic construct
  misc_feature 1..80: note = Synthetic aptamer sequences
  source 1..80: mol_type = other RNA
gggtgcatcgtttacgcgtaaaaataacaaacttctcagctgtttaatatctcctgactgctgctgaggaaggatatgag

SEQ ID NO: 86 - RNA, 80 nt, synthetic construct
  misc_feature 1..80: note = Synthetic aptamer sequences
  source 1..80: mol_type = other RNA
gggtgcatcgtttacgcgtaaaattaaacaaatagctcagcacgaaaatttgcgtaactgctgctgaggaaggatatgag

SEQ ID NO: 87 - RNA, 80 nt, synthetic construct
  misc_feature 1..80: note = Synthetic aptamer sequences
  source 1..80: mol_type = other RNA
gggtgcatcgtttacgcgtaattaaaaaaccttcacacagaaaacattcctcaatttctgctgctgaggaaggatatgag

SEQ ID NO: 88 - RNA, 80 nt, synthetic construct
  misc_feature 1..80: note = Synthetic aptamer sequences
  source 1..80: mol_type = other RNA
gggtgcatcgtttacgcattttcgttttattttagtttaattgcgtttagtatctggctgctgctgaggaaggatatgag

SEQ ID NO: 89 - RNA, 80 nt, synthetic construct
  misc_feature 1..80: note = Synthetic aptamer sequences
  source 1..80: mol_type = other RNA
gggtgcatcgtttacgcgtaaaaagtataaaggttagaaattcagcagtttgatatcctgctgctgaggaaggatatgag

SEQ ID NO: 90 - RNA, 80 nt, synthetic construct
  misc_feature 1..80: note = Synthetic aptamer sequences
  source 1..80: mol_type = other RNA
gggtgcatcgtttacgcgtaaaaaggagaattagtactcaccagtcgtttaaaatttctgctgctgaggaaggatatgag

SEQ ID NO: 91 - RNA, 80 nt, synthetic construct
  misc_feature 1..80: note = Synthetic aptamer sequences
  source 1..80: mol_type = other RNA
gggtgcatcgtttacgcgtaaaaataaataactacgagatctcagcagatcattatcctgctgctgaggaaggatatgag

SEQ ID NO: 92 - RNA, 80 nt, synthetic construct
  misc_feature 1..80: note = Synthetic aptamer sequences
  source 1..80: mol_type = other RNA
gggtgcatcgtttacgcgtaaaatggtttttcagcagttaacataatgcctcagtttctgctgctgaggaaggatatgag

SEQ ID NO: 93 - RNA, 80 nt, synthetic construct
  misc_feature 1..80: note = Synthetic aptamer sequences
  source 1..80: mol_type = other RNA
gggtgcatcgtttacgcgtaaataacaaaaatctcagcttttgcagaatttatccacctgctgctgaggaaggatatgag

SEQ ID NO: 94 - RNA, 80 nt, synthetic construct
  misc_feature 1..80: note = Synthetic aptamer sequences
  source 1..80: mol_type = other RNA
gggtgcatcgtttacgcgtaaataaactcacagcagaaaaaattccttcaacttgtactgctgctgaggaaggatatgag

SEQ ID NO: 95 - RNA, 80 nt, synthetic construct
  misc_feature 1..80: note = Synthetic aptamer sequences
  source 1..80: mol_type = other RNA
gggtgcatcgtttacgcagtagttaataacaaatagtcagcagttttgtccttcattctgctgctgaggaaggatatgag

SEQ ID NO: 96 - RNA, 80 nt, synthetic construct
  misc_feature 1..80: note = Synthetic aptamer sequences
  source 1..80: mol_type = other RNA
gggtgcatcgtttacgcgtaaaaatagcagtagatagcggcagttttgtatttgttactgctgctgaggaaggatatgag

SEQ ID NO: 97 - RNA, 80 nt, synthetic construct
  misc_feature 1..80: note = Synthetic aptamer sequences
  source 1..80: mol_type = other RNA
gggtgcatcgtttacgcgtaaaaatttaaataactcagcaatcatagatccgactgactgctgctgaggaaggatatgag

SEQ ID NO: 98 - RNA, 80 nt, synthetic construct
  misc_feature 1..80: note = Synthetic aptamer sequences
  source 1..80: mol_type = other RNA
gggtgcatcgtttacgcgtaaagaacagctgacaagaaattcaaaccttcagattttctgctgctgaggaaggatatgag

SEQ ID NO: 99 - RNA, 80 nt, synthetic construct
  misc_feature 1..80: note = Synthetic aptamer sequences
  source 1..80: mol_type = other RNA
gggtgcatcgtttacgcgtaaagataataagcagtattcagcagatttgtaaggtttctgctgctgaggaaggatatgag

SEQ ID NO: 100 - RNA, 80 nt, synthetic construct
  misc_feature 1..80: note = Synthetic aptamer sequences
  source 1..80: mol_type = other RNA
gggtgcatcgtttacgcgtaaataagaggcagacagtattacaaatatcctaaaatactgctgctgaggaaggatatgag

SEQ ID NO: 101 - DNA, 80 bp, synthetic construct
  misc_feature 1..80: note = Synthetic aptamer sequences
  source 1..80: mol_type = other DNA
gggtgcatcgtttacgcgattagtctgataaacaaaaagatttcgctaaaaatcaatctgctgctgaggaaggatatgag

SEQ ID NO: 102 - DNA, 80 bp, synthetic construct
  misc_feature 1..80: note = Synthetic aptamer sequences
  source 1..80: mol_type = other DNA
gggtgcatcgtttacgcagatagcagcaggaatcaagcggtaggagtctagcagaagctgctgctgaggaaggatatgag

SEQ ID NO: 103 - DNA, 80 bp, synthetic construct
  misc_feature 1..80: note = Synthetic aptamer sequences
  source 1..80: mol_type = other DNA
gggtgcatcgtttacgcattttcgttttatttcagtttaattgcgtttagtatctggctgctgctgaggaaggatatgag

SEQ ID NO: 104 - DNA, 80 bp, synthetic construct
  misc_feature 1..80: note = Synthetic aptamer sequences
  source 1..80: mol_type = other DNA
gggtgcatcgtttacgcgcaacataaaaatttaaagtgctcagttgtcaatctatgactgctgctgaggaaggatatgag

SEQ ID NO: 105 - DNA, 80 bp, synthetic construct
  misc_feature 1..80: note = Synthetic aptamer sequences
  source 1..80: mol_type = other DNA
gggtgcatcgtttacgcgtaaatggtccgctattaaaagaaaagaatgaagtctcagctgctgctgaggaaggatatgag

SEQ ID NO: 106 - DNA, 80 bp, synthetic construct
  misc_feature 1..80: note = Synthetic aptamer sequences
  source 1..80: mol_type = other DNA
gggtgcatcgtttacgctattttcatttgtttttttaatttactagtgtaaacaatcctgctgctgaggaaggatatgag

SEQ ID NO: 107 - DNA, 80 bp, synthetic construct
  misc_feature 1..80: note = Synthetic aptamer sequences
  source 1..80: mol_type = other DNA
gggtgcatcgtttacgcgtaaataagtagataaagtggcagtttgttttccttggaactgctgctgaggaaggatatgag

SEQ ID NO: 108 - DNA, 80 bp, synthetic construct
  misc_feature 1..80: note = Synthetic aptamer sequences
  source 1..80: mol_type = other DNA
gggtgcatcgtttacgcgtaaaaattaaagagattaaggtccttaagcagttttgtcctgctgctgaggaaggatatgag

SEQ ID NO: 109 - DNA, 80 bp, synthetic construct
  misc_feature 1..80: note = Synthetic aptamer sequences
  source 1..80: mol_type = other DNA
gggtgcatcgtttacgcgtaaaaaaatcaaaacttcagcaaattatttatcaacgtcctgctgctgaggaaggatatgag

SEQ ID NO: 110 - DNA, 80 bp, synthetic construct
  misc_feature 1..80: note = Synthetic aptamer sequences
  source 1..80: mol_type = other DNA
gggtgcatcgtttacgcgtaaaataaattaaaaagaacttcttcagcaatcaatatcctgctgctgaggaaggatatgag

SEQ ID NO: 111 - DNA, 80 bp, synthetic construct
  misc_feature 1..80: note = Synthetic aptamer sequences
  source 1..80: mol_type = other DNA
gggtgcatcgtttacgcgtaaataaaaatgaaaaattgtctctcagctttcaaagtcctgctgctgaggaaggatatgag

SEQ ID NO: 112 - DNA, 80 bp, synthetic construct
  misc_feature 1..80: note = Synthetic aptamer sequences
  source 1..80: mol_type = other DNA
gggtgcatcgtttacgcgtaaaaaaaaaatatcttcggagaattcagcaattttatcctgctgctgaggaaggatatgag

SEQ ID NO: 113 - DNA, 80 bp, synthetic construct
  misc_feature 1..80: note = Synthetic aptamer sequences
  source 1..80: mol_type = other DNA
gggtgcatcgtttacgcgtaaaaattttcatctcagcaattaaatccaaagaatccactgctgctgaggaaggatatgag

SEQ ID NO: 114 - DNA, 80 bp, synthetic construct
  misc_feature 1..80: note = Synthetic aptamer sequences
  source 1..80: mol_type = other DNA
gggtgcatcgtttacgcgtaaaatatatcagcaaagtagtttaagcctcctcagtttctgctgctgaggaaggatatgag

SEQ ID NO: 115 - DNA, 80 bp, synthetic construct
  misc_feature 1..80: note = Synthetic aptamer sequences
  source 1..80: mol_type = other DNA
gggtgcatcgtttacgcgtaaattatgaaaaatacagcaaggatttaacctcagtttctgctgctgaggaaggatatgag

SEQ ID NO: 116 - DNA, 80 bp, synthetic construct
  misc_feature 1..80: note = Synthetic aptamer sequences
  source 1..80: mol_type = other DNA
gggtgcatcgtttacgcgtaaaataaataaatcttcaaagtacagacctcgatttttctgctgctgaggaaggatatgag

SEQ ID NO: 117 - DNA, 80 bp, synthetic construct
  misc_feature 1..80: note = Synthetic aptamer sequences
  source 1..80: mol_type = other DNA
gggtgcatcgtttacgcttataggtattagacattttcaattaaagtgaattagtgtctgctgctgaggaaggatatgag

SEQ ID NO: 118 - DNA, 80 bp, synthetic construct
  misc_feature 1..80: note = Synthetic aptamer sequences
  source 1..80: mol_type = other DNA
gggtgcatcgtttacgcgtaaaatgtgacagcaggataataaaataagtactcagtactgctgctgaggaaggatatgag

SEQ ID NO: 119 - DNA, 80 bp, synthetic construct
  misc_feature 1..80: note = Synthetic aptamer sequences
  source 1..80: mol_type = other DNA
gggtgcatcgtttacgcgtaattaagaaaaataaaagtactctgcagtttttatccactgctgctgaggaaggatatgag

SEQ ID NO: 120 - DNA, 80 bp, synthetic construct
  misc_feature 1..80: note = Synthetic aptamer sequences
  source 1..80: mol_type = other DNA
gggtgcatcgtttacgcgtaaaaataaaattttcccagaccagttatctgccttaaactgctgctgaggaaggatatgag

SEQ ID NO: 121 - DNA, 80 bp, synthetic construct
  misc_feature 1..80: note = Synthetic aptamer sequences
  source 1..80: mol_type = other DNA
gggtgcatcgtttacgcgtaaagaaaaaaatcagcttttagtcgccttccattttgactgctgctgaggaaggatatgag

SEQ ID NO: 122 - DNA, 80 bp, synthetic construct
  misc_feature 1..80: note = Synthetic aptamer sequences
  source 1..80: mol_type = other DNA
gggtgcatcgtttacgcgtaaataaataatcaaaattacactcagtggcaatttcctctgctgctgaggaaggatatgag

SEQ ID NO: 123 - DNA, 80 bp, synthetic construct
  misc_feature 1..80: note = Synthetic aptamer sequences
  source 1..80: mol_type = other DNA
gggtgcatcgtttacgcgtaaaatacaggatacgacaataactcagcagattttatcctgctgctgaggaaggatatgag

SEQ ID NO: 124 - DNA, 80 bp, synthetic construct
  misc_feature 1..80: note = Synthetic aptamer sequences
  source 1..80: mol_type = other DNA
gggtgcatcgtttacgcgttaaaaattgtgcactgagatgacgcagcattaactacactgctgctgaggaaggatatgag

SEQ ID NO: 125 - DNA, 80 bp, synthetic construct
  misc_feature 1..80: note = Synthetic aptamer sequences
  source 1..80: mol_type = other DNA
gggtgcatcgtttacgcgtaaataaaaattaatcagcaattttccactcagttgtacctgctgctgaggaaggatatgag

SEQ ID NO: 126 - DNA, 80 bp, synthetic construct
  misc_feature 1..80: note = Synthetic aptamer sequences
  source 1..80: mol_type = other DNA
gggtgcatcgtttacgcgtaaaaataaaaaatctcgatcactgcagttttattccggctgctgctgaggaaggatatgag

SEQ ID NO: 127 - DNA, 80 bp, synthetic construct
  misc_feature 1..80: note = Synthetic aptamer sequences
  source 1..80: mol_type = other DNA
gggtgcatcgtttacgcgtaaacaaatatcgattaaaataaaatctcagcaagaatcctgctgctgaggaaggatatgag

SEQ ID NO: 128 - DNA, 80 bp, synthetic construct
  misc_feature 1..80: note = Synthetic aptamer sequences
  source 1..80: mol_type = other DNA
gggtgcatcgtttacgcgtaaaataaataaaattatcccaggagcaaattttcttcgctgctgctgaggaaggatatgag

SEQ ID NO: 129 - DNA, 80 bp, synthetic construct
  misc_feature 1..80: note = Synthetic aptamer sequences
  source 1..80: mol_type = other DNA
gggtgcatcgtttacgcgtagaagaattaatagtggacatatcaatagcagtttatcctgctgctgaggaaggatatgag

SEQ ID NO: 130 - DNA, 80 bp, synthetic construct
  misc_feature 1..80: note = Synthetic aptamer sequences
  source 1..80: mol_type = other DNA
gggtgcatcgtttacgcgtaaacatattcagcagttaaaatttagtaggttcagtagctgctgctgaggaaggatatgag

SEQ ID NO: 131 - DNA, 80 bp, synthetic construct
  misc_feature 1..80: note = Synthetic aptamer sequences
  source 1..80: mol_type = other DNA
gggtgcatcgtttacgcgtaaaaaagataaaacttagttgcagaatttgccttcattctgctgctgaggaaggatatgag

SEQ ID NO: 132 - DNA, 80 bp, synthetic construct
  misc_feature 1..80: note = Synthetic aptamer sequences
  source 1..80: mol_type = other DNA
gggtgcatcgtttacgcgtaaaaagtttgatggaagcagattagtttagtcaaatttctgctgctgaggaaggatatgag

SEQ ID NO: 133 - DNA, 80 bp, synthetic construct
  misc_feature 1..80: note = Synthetic aptamer sequences
  source 1..80: mol_type = other DNA
gggtgcatcgtttacgcgtaaaatgaaataaggaatccttcagcagtatttatccttctgctgctgaggaaggatatgag

SEQ ID NO: 134 - DNA, 80 bp, synthetic construct
  misc_feature 1..80: note = Synthetic aptamer sequences
  source 1..80: mol_type = other DNA
gggtgcatcgtttacgcgtaaagaataaaaatgacaaaattctcagcttttgtcaacctgctgctgaggaaggatatgag

SEQ ID NO: 135 - DNA, 80 bp, synthetic construct
  misc_feature 1..80: note = Synthetic aptamer sequences
  source 1..80: mol_type = other DNA
gggtgcatcgtttacgcgtaaaaaatgaaatgaaaaaattctcagctgtctatcttcctgctgctgaggaaggatatgag

SEQ ID NO: 136 - DNA, 80 bp, synthetic construct
  misc_feature 1..80: note = Synthetic aptamer sequences
  source 1..80: mol_type = other DNA
gggtgcatcgtttacgcgtaaataagtaaaaaactcagttttcagttaagtatccaactgctgctgaggaaggatatgag

SEQ ID NO: 137 - DNA, 80 bp, synthetic construct
  misc_feature 1..80: note = Synthetic aptamer sequences
  source 1..80: mol_type = other DNA
gggtgcatcgtttacgcgtaaatttcagcagagtaataataacacttcttcagtttgctgctgctgaggaaggatatgag

SEQ ID NO: 138 - DNA, 80 bp, synthetic construct
  misc_feature 1..80: note = Synthetic aptamer sequences
  source 1..80: mol_type = other DNA
gggtgcatcgtttacgcgtaaaattaagaagtattatcagttagctttttcttccaactgctgctgaggaaggatatgag

SEQ ID NO: 139 - DNA, 80 bp, synthetic construct
  misc_feature 1..80: note = Synthetic aptamer sequences
  source 1..80: mol_type = other DNA
gggtgcatcgtttacgcgtaaaataaaaagttttcctatcagcaaactcacaaattcctgctgctgaggaaggatatgag

SEQ ID NO: 140 - DNA, 80 bp, synthetic construct
  misc_feature 1..80: note = Synthetic aptamer sequences
  source 1..80: mol_type = other DNA
gggtgcatcgtttacgcgtaaaatgaaatgtaaaagaattgaacttggcagattttcctgctgctgaggaaggatatgag

SEQ ID NO: 141 - DNA, 80 bp, synthetic construct
  misc_feature 1..80: note = Synthetic aptamer sequences
  source 1..80: mol_type = other DNA
gggtgcatcgtttacgcgtaaattaaagtagcagtaatttcagcagtttttacctctctgctgctgaggaaggatatgag

SEQ ID NO: 142 - DNA, 80 bp, synthetic construct
  misc_feature 1..80: note = Synthetic aptamer sequences
  source 1..80: mol_type = other DNA
gggtgcatcgtttacgcgtaaataaaggataaaataatttcagggcagtttctcatcctgctgctgaggaaggatatgag

SEQ ID NO: 143 - DNA, 80 bp, synthetic construct
  misc_feature 1..80: note = Synthetic aptamer sequences
  source 1..80: mol_type = other DNA
gggtgcatcgtttacgcaggatcgttttaagtaaaataaaagatttccttggtaatcctgctgctgaggaaggatatgag

SEQ ID NO: 144 - DNA, 80 bp, synthetic construct
  misc_feature 1..80: note = Synthetic aptamer sequences
  source 1..80: mol_type = other DNA
gggtgcatcgtttacgcgtaaaataaagatcaattaaaggctttgatcgattttcctctgctgctgaggaaggatatgag

SEQ ID NO: 145 - DNA, 80 bp, synthetic construct
  misc_feature 1..80: note = Synthetic aptamer sequences
  source 1..80: mol_type = other DNA
gggtgcatcgtttacgcgtaaaaattagagattaaaatagttcctttcagttttgtcctgctgctgaggaaggatatgag

SEQ ID NO: 146 - DNA, 80 bp, synthetic construct
  misc_feature 1..80: note = Synthetic aptamer sequences
  source 1..80: mol_type = other DNA
gggtgcatcgtttacgcgtaaaattgacaatgtgaaaagcagacagcaaatattcctctgctgctgaggaaggatatgag

SEQ ID NO: 147 - DNA, 80 bp, synthetic construct
  misc_feature 1..80: note = Synthetic aptamer sequences
  source 1..80: mol_type = other DNA
gggtgcatcgtttacgcgtaaataaccagttatacagaaagatctcagcaatttatcctgctgctgaggaaggatatgag

SEQ ID NO: 148 - DNA, 80 bp, synthetic construct
  misc_feature 1..80: note = Synthetic aptamer sequences
  source 1..80: mol_type = other DNA
gggtgcatcgtttacgcttacagaaggattgcaccacatgcgtactcgatgaaacacctgctgctgaggaaggatatgag

SEQ ID NO: 149 - DNA, 80 bp, synthetic construct
  misc_feature 1..80: note = Synthetic aptamer sequences
  source 1..80: mol_type = other DNA
gggtgcatcgtttacgcgtaaaataataattaaactcagcaaattcaatccaactttctgctgctgaggaaggatatgag

SEQ ID NO: 150 - DNA, 80 bp, synthetic construct
  misc_feature 1..80: note = Synthetic aptamer sequences
  source 1..80: mol_type = other DNA
gggtgcatcgtttacgcgtaaacaagaataaattcagcagtggttttgatcctttgactgctgctgaggaaggatatgag

SEQ ID NO: 151 - DNA, 80 bp, synthetic construct
  misc_feature 1..80: note = Synthetic aptamer sequences
  source 1..80: mol_type = other DNA
gggtgcatcgtttacgcgtaaattaatcagattgaacaaaagttttccctcagttttctgctgctgaggaaggatatgag

SEQ ID NO: 152 - DNA, 80 bp, synthetic construct
  misc_feature 1..80: note = Synthetic aptamer sequences
  source 1..80: mol_type = other DNA
gggtgcatcgtttacgcgtaaagaaaaacatcagagcagttataatagtcctttttcctgctgctgaggaaggatatgag

SEQ ID NO: 153 - DNA, 80 bp, synthetic construct
  misc_feature 1..80: note = Synthetic aptamer sequences
  source 1..80: mol_type = other DNA
gggtgcatcgtttacgcgtaaagaaaataaacttgatcaaacttagcagtttttatcctgctgctgaggaaggatatgag

SEQ ID NO: 154 - DNA, 80 bp, synthetic construct
  misc_feature 1..80: note = Synthetic aptamer sequences
  source 1..80: mol_type = other DNA
gggtgcatcgtttacgcattttcgttatatttctggtttttatgcgtgagaatcctgctgctgctgaggaaggatatgag

SEQ ID NO: 155 - DNA, 80 bp, synthetic construct
  misc_feature 1..80: note = Synthetic aptamer sequences
  source 1..80: mol_type = other DNA
gggtgcatcgtttacgcgtaaaaataagatctcacagcgacaaatttttcttccagtctgctgctgaggaaggatatgag

SEQ ID NO: 156 - DNA, 80 bp, synthetic construct
  misc_feature 1..80: note = Synthetic aptamer sequences
  source 1..80: mol_type = other DNA
gggtgcatcgtttacgcgtaaatttaagacatgacagcagacattttatcttcagacctgctgctgaggaaggatatgag

SEQ ID NO: 157 - DNA, 80 bp, synthetic construct
  misc_feature 1..80: note = Synthetic aptamer sequences
  source 1..80: mol_type = other DNA
gggtgcatcgtttacgcgtaataacagaaatataactcagctgaattaatttttccgctgctgctgaggaaggatatgag

SEQ ID NO: 158 - DNA, 80 bp, synthetic construct
  misc_feature 1..80: note = Synthetic aptamer sequences
  source 1..80: mol_type = other DNA
gggtgcatcgtttacgcgtaaaaataaattccaaaatattcagcagaaatcctcgaactgctgctgaggaaggatatgag

SEQ ID NO: 159 - DNA, 80 bp, synthetic construct
  misc_feature 1..80: note = Synthetic aptamer sequences
  source 1..80: mol_type = other DNA
gggtgcatcgtttacgcgtaaaaataataggttccaatcaagcagtacaaaattcctctgctgctgaggaaggatatgag

SEQ ID NO: 160 - DNA, 80 bp, synthetic construct
  misc_feature 1..80: note = Synthetic aptamer sequences
  source 1..80: mol_type = other DNA
gggtgcatcgtttacgcgtaaaaaatctaaaaagatatcagcaggcaaattttccttctgctgctgaggaaggatatgag

SEQ ID NO: 161 - DNA, 80 bp, synthetic construct
  misc_feature 1..80: note = Synthetic aptamer sequences
  source 1..80: mol_type = other DNA
gggtgcatcgtttacgcgtaaaataaagaggataactacaatcatcagcaatcatatctgctgctgaggaaggatatgag

SEQ ID NO: 162 - DNA, 80 bp, synthetic construct
  misc_feature 1..80: note = Synthetic aptamer sequences
  source 1..80: mol_type = other DNA
gggtgcatcgtttacgcgtaaatttagtagaaaggaaagacgaagtttcctcagtttctgctgctgaggaaggatatgag

SEQ ID NO: 163 - DNA, 80 bp, synthetic construct
  misc_feature 1..80: note = Synthetic aptamer sequences
  source 1..80: mol_type = other DNA
gggtgcatcgtttacgcgtaaaaataatagatctcagaatatgaaagcagttctttcctgctgctgaggaaggatatgag

SEQ ID NO: 164 - DNA, 80 bp, synthetic construct
  misc_feature 1..80: note = Synthetic aptamer sequences
  source 1..80: mol_type = other DNA
gggtgcatcgtttacgcgtaacaagatattcacagcagattttaaaaaattcctcgtctgctgctgaggaaggatatgag

SEQ ID NO: 165 - DNA, 80 bp, synthetic construct
  misc_feature 1..80: note = Synthetic aptamer sequences
  source 1..80: mol_type = other DNA
gggtgcatcgtttacgcgtaaaaagttgacaattaataaaatcttcttagcattttcctgctgctgaggaaggatatgag

SEQ ID NO: 166 - DNA, 80 bp, synthetic construct
  misc_feature 1..80: note = Synthetic aptamer sequences
  source 1..80: mol_type = other DNA
gggtgcatcgtttacgcgtaaaacaaaatgaaacttatagctcagcatattttgatcctgctgctgaggaaggatatgag

SEQ ID NO: 167 - DNA, 80 bp, synthetic construct
  misc_feature 1..80: note = Synthetic aptamer sequences
  source 1..80: mol_type = other DNA
gggtgcatcgtttacgcgtaaattatcaaaaaagcagatttaagtatacctcagttactgctgctgaggaaggatatgag

SEQ ID NO: 168 - DNA, 80 bp, synthetic construct
  misc_feature 1..80: note = Synthetic aptamer sequences
  source 1..80: mol_type = other DNA
gggtgcatcgtttacgcgtaaataaaatagctcagcaaggaagtttttttcctcaaactgctgctgaggaaggatatgag

SEQ ID NO: 169 - DNA, 80 bp, synthetic construct
  misc_feature 1..80: note = Synthetic aptamer sequences
  source 1..80: mol_type = other DNA
gggtgcatcgtttacgcgtaaatttgagaaaagaacagcagactcaaatctttttaactgctgctgaggaaggatatgag

SEQ ID NO: 170 - DNA, 80 bp, synthetic construct
  misc_feature 1..80: note = Synthetic aptamer sequences
  source 1..80: mol_type = other DNA
gggtgcatcgtttacgcgtaacagaaaattaagctcagcaatagtaattatcctagtctgctgctgaggaaggatatgag

SEQ ID NO: 171 - DNA, 80 bp, synthetic construct
  misc_feature 1..80: note = Synthetic aptamer sequences
  source 1..80: mol_type = other DNA
gggtgcatcgtttacgcgtaatgaaaataaatcagtctcacagcattttaaaacttcctgctgctgaggaaggatatgag

SEQ ID NO: 172 - DNA, 80 bp, synthetic construct
  misc_feature 1..80: note = Synthetic aptamer sequences
  source 1..80: mol_type = other DNA
gggtgcatcgtttacgcgtatttacaagcaacaaagttacaatcagcagaatttatcctgctgctgaggaaggatatgag

SEQ ID NO: 173 - DNA, 80 bp, synthetic construct
  misc_feature 1..80: note = Synthetic aptamer sequences
  source 1..80: mol_type = other DNA
gggtgcatcgtttacgcgtaaaaaattgtctatagcacttttagattcccaaactaactgctgctgaggaaggatatgag

SEQ ID NO: 174 - DNA, 80 bp, synthetic construct
  misc_feature 1..80: note = Synthetic aptamer sequences
  source 1..80: mol_type = other DNA
gggtgcatcgtttacgcgtaaaaaaatcagcaaaatcgaaaactcatgcagtttgtcctgctgctgaggaaggatatgag

SEQ ID NO: 175 - DNA, 80 bp, synthetic construct
  misc_feature 1..80: note = Synthetic aptamer sequences
  source 1..80: mol_type = other DNA
gggtgcatcgtttacgcgtaaaaaattccttaaaaatttaactaactggataggtctctgctgctgaggaaggatatgag

SEQ ID NO: 176 - DNA, 80 bp, synthetic construct
  misc_feature 1..80: note = Synthetic aptamer sequences
  source 1..80: mol_type = other DNA
gggtgcatcgtttacgcgtaaaacaaaatttctgacagcaattccttcgttaaaaatctgctgctgaggaaggatatgag

SEQ ID NO: 177 - DNA, 80 bp, synthetic construct
  misc_feature 1..80: note = Synthetic aptamer sequences
  source 1..80: mol_type = other DNA
gggtgcatcgtttacgcgtaaattattaaaaaaatcagcaaagtttatttcccacggctgctgctgaggaaggatatgag

SEQ ID NO: 178 - DNA, 80 bp, synthetic construct
  misc_feature 1..80: note = Synthetic aptamer sequences
  source 1..80: mol_type = other DNA
gggtgcatcgtttacgcgtaattaatcaaacaatagcagcaaatctcagcaattttcctgctgctgaggaaggatatgag

SEQ ID NO: 179 - DNA, 80 bp, synthetic construct
  misc_feature 1..80: note = Synthetic aptamer sequences
  source 1..80: mol_type = other DNA
gggtgcatcgtttacgcgtaatttgaaagtctcataaatttttttttttttttcaatctgctgctgaggaaggatatgag

SEQ ID NO: 180 - DNA, 80 bp, synthetic construct
  misc_feature 1..80: note = Synthetic aptamer sequences
  source 1..80: mol_type = other DNA
gggtgcatcgtttacgcgtaaaaattcagcatgatttcaattactcctttcattgatctgctgctgaggaaggatatgag

SEQ ID NO: 181 - DNA, 80 bp, synthetic construct
  misc_feature 1..80: note = Synthetic aptamer sequences
  source 1..80: mol_type = other DNA
gggtgcatcgtttacgcgtaaaataaataaaaatcagtagcaatctttctcacagtgctgctgctgaggaaggatatgag

SEQ ID NO: 182 - DNA, 80 bp, synthetic construct
  misc_feature 1..80: note = Synthetic aptamer sequences
  source 1..80: mol_type = other DNA
gggtgcatcgtttacgcgtaaataaaaagcagatctcagcaaaactcgtaaattcaactgctgctgaggaaggatatgag

SEQ ID NO: 183 - DNA, 80 bp, synthetic construct
  misc_feature 1..80: note = Synthetic aptamer sequences
  source 1..80: mol_type = other DNA
gggtgcatcgtttacgcgtaaataatgaaggactcagacagttaaaagatgcattaactgctgctgaggaaggatatgag

SEQ ID NO: 184 - DNA, 80 bp, synthetic construct
  misc_feature 1..80: note = Synthetic aptamer sequences
  source 1..80: mol_type = other DNA
gggtgcatcgtttacgcgtaaaaaagatcaatatgaaaatcagcagttaatatcttcctgctgctgaggaaggatatgag

SEQ ID NO: 185 - DNA, 80 bp, synthetic construct
  misc_feature 1..80: note = Synthetic aptamer sequences
  source 1..80: mol_type = other DNA
gggtgcatcgtttacgcgtaaaaataacaaacttctcagctgtttaatatctcctgactgctgctgaggaaggatatgag

SEQ ID NO: 186 - DNA, 80 bp, synthetic construct
  misc_feature 1..80: note = Synthetic aptamer sequences
  source 1..80: mol_type = other DNA
gggtgcatcgtttacgcgtaaaattaaacaaatagctcagcacgaaaatttgcgtaactgctgctgaggaaggatatgag

SEQ ID NO: 187 - DNA, 80 bp, synthetic construct
  misc_feature 1..80: note = Synthetic aptamer sequences
  source 1..80: mol_type = other DNA
gggtgcatcgtttacgcgtaattaaaaaaccttcacacagaaaacattcctcaatttctgctgctgaggaaggatatgag

SEQ ID NO: 188 - DNA, 80 bp, synthetic construct
  misc_feature 1..80: note = Synthetic aptamer sequences
  source 1..80: mol_type = other DNA
gggtgcatcgtttacgcattttcgttttattttagtttaattgcgtttagtatctggctgctgctgaggaaggatatgag

SEQ ID NO: 189 - DNA, 80 bp, synthetic construct
  misc_feature 1..80: note = Synthetic aptamer sequences
  source 1..80: mol_type = other DNA
gggtgcatcgtttacgcgtaaaaagtataaaggttagaaattcagcagtttgatatcctgctgctgaggaaggatatgag

SEQ ID NO: 190 - DNA, 80 bp, synthetic construct
  misc_feature 1..80: note = Synthetic aptamer sequences
  source 1..80: mol_type = other DNA
gggtgcatcgtttacgcgtaaaaaggagaattagtactcaccagtcgtttaaaatttctgctgctgaggaaggatatgag

SEQ ID NO: 191 - DNA, 80 bp, synthetic construct
  misc_feature 1..80: note = Synthetic aptamer sequences
  source 1..80: mol_type = other DNA
gggtgcatcgtttacgcgtaaaaataaataactacgagatctcagcagatcattatcctgctgctgaggaaggatatgag

SEQ ID NO: 192 - DNA, 80 bp, synthetic construct
  misc_feature 1..80: note = Synthetic aptamer sequences
  source 1..80: mol_type = other DNA
gggtgcatcgtttacgcgtaaaatggtttttcagcagttaacataatgcctcagtttctgctgctgaggaaggatatgag

SEQ ID NO: 193 - DNA, 80 bp, synthetic construct
  misc_feature 1..80: note = Synthetic aptamer sequences
  source 1..80: mol_type = other DNA
gggtgcatcgtttacgcgtaaataacaaaaatctcagcttttgcagaatttatccacctgctgctgaggaaggatatgag

SEQ ID NO: 194 - DNA, 80 bp, synthetic construct
  misc_feature 1..80: note = Synthetic aptamer sequences
  source 1..80: mol_type = other DNA
gggtgcatcgtttacgcgtaaataaactcacagcagaaaaaattccttcaacttgtactgctgctgaggaaggatatgag

SEQ ID NO: 195 - DNA, 80 bp, synthetic construct
  misc_feature 1..80: note = Synthetic aptamer sequences
  source 1..80: mol_type = other DNA
gggtgcatcgtttacgcagtagttaataacaaatagtcagcagttttgtccttcattctgctgctgaggaaggatatgag

SEQ ID NO: 196 - DNA, 80 bp, synthetic construct
  misc_feature 1..80: note = Synthetic aptamer sequences
  source 1..80: mol_type = other DNA
gggtgcatcgtttacgcgtaaaaatagcagtagatagcggcagttttgtatttgttactgctgctgaggaaggatatgag

SEQ ID NO: 197 - DNA, 80 bp, synthetic construct
  misc_feature 1..80: note = Synthetic aptamer sequences
  source 1..80: mol_type = other DNA
gggtgcatcgtttacgcgtaaaaatttaaataactcagcaatcatagatccgactgactgctgctgaggaaggatatgag

SEQ ID NO: 198 - DNA, 80 bp, synthetic construct
  misc_feature 1..80: note = Synthetic aptamer sequences
  source 1..80: mol_type = other DNA
gggtgcatcgtttacgcgtaaagaacagctgacaagaaattcaaaccttcagattttctgctgctgaggaaggatatgag

SEQ ID NO: 199 - DNA, 80 bp, synthetic construct
  misc_feature 1..80: note = Synthetic aptamer sequences
  source 1..80: mol_type = other DNA
gggtgcatcgtttacgcgtaaagataataagcagtattcagcagatttgtaaggtttctgctgctgaggaaggatatgag

SEQ ID NO: 200 - DNA, 80 bp, synthetic construct
  misc_feature 1..80: note = Synthetic aptamer sequences
  source 1..80: mol_type = other DNA
gggtgcatcgtttacgcgtaaataagaggcagacagtattacaaatatcctaaaatactgctgctgaggaaggatatgag

SEQ ID NO: 201 - RNA, 11 nt, synthetic construct
  misc_feature 1..11: note = Synthetic aptamer sequences
  source 1..11: mol_type = other RNA
aaacaaaaaga

SEQ ID NO: 202 - skipped

SEQ ID NO: 203 - DNA, 11 bp, synthetic construct
  misc_feature 1..11: note = Synthetic aptamer sequences
  source 1..11: mol_type = other DNA
aaacaaaaaga

SEQ ID NO: 204 - skipped

SEQ ID NO: 205 - RNA, 13 nt, synthetic construct
  misc_feature 1..13: note = Synthetic aptamer sequences
  source 1..13: mol_type = other RNA
ataaaaatttaaa

SEQ ID NO: 206 - DNA, 13 bp, synthetic construct
  misc_feature 1..13: note = Synthetic aptamer sequences
  source 1..13: mol_type = other DNA
ataaaaatttaaa

SEQ ID NO: 207 - RNA, 12 nt, synthetic construct
  misc_feature 1..12: note = Synthetic aptamer sequences
  source 1..12: mol_type = other RNA
gtaaaaattaaa

SEQ ID NO: 208 - DNA, 12 bp, synthetic construct
  misc_feature 1..12: note = Synthetic aptamer sequences
  source 1..12: mol_type = other DNA
gtaaaaattaaa

SEQ ID NO: 209-212 - skipped (4 entries)

SEQ ID NO: 213 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 214 - protein, 453 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYE

SEQ ID NO: 215 - protein, 63 aa, Homo sapiens
GGSVLVTCSTSCDQPKLLGIETPLPKKELLLPGNNRKVYELSNVQEDSQPMCYSNCPDGQSTA

SEQ ID NO: 216 - protein, 66 aa, Homo sapiens
GKNLTLRCQVEGGAPRANLTVVLLRGEKELKREPAVGEPAEVTTTVLVRRDHHGANFSCRTELDLR

SEQ ID NO: 217 - protein, 68 aa, Homo sapiens
DTQGTVVCSLDGLFPVSEAQVHLALGDQRLNPTVTYGNDSFSAKASVSVTAEDEGTQRLTCAVILGNQ

SEQ ID NO: 218 - protein, 54 aa, Homo sapiens
GTEVTVKCEAHPRAKVTLNGVPAQPLGPRAQLLLKATPEDNGRSFSCSATLEVA

SEQ ID NO: 219 - protein, 53 aa, Homo sapiens
NSQQTPMCQAWGNPLPELKCLKDGTFPLPIGESVTVTRDLEGTYLCRARSTQG

SEQ ID NO: 220 - protein, 13 aa, Homo sapiens
QTSVSPSKVILPR

SEQ ID NO: 221 - protein, 10 aa, Homo sapiens
SCDQPKLLGI

SEQ ID NO: 222 - protein, 16 aa, Homo sapiens
PKKELLLPGNNRKVYE

SEQ ID NO: 223 - protein, 15 aa, Homo sapiens
YSNCPDGQSTAKTFL

SEQ ID NO: 224 - DNA, 17 bp, synthetic construct
  misc_feature 1..17: note = Primer
  source 1..17: mol_type = other DNA
gggtgcatcgtttacgc

SEQ ID NO: 225 - DNA, 23 bp, synthetic construct
  misc_feature 1..23: note = Primer
  source 1..23: mol_type = other DNA
ctgctgctgaggaaggatatgag

SEQ ID NO: 226 - DNA, 34 bp, synthetic construct
  misc_feature 1..34: note = Primer
  source 1..34: mol_type = other DNA
taatacgactcactatagggtgcatcgtttacgc

SEQ ID NO: 227 - DNA, 23 bp, synthetic construct
  misc_feature 1..23: note = Primer
  source 1..23: mol_type = other DNA
ctcatatccttcctcagcagcag

SEQ ID NO: 228 - protein, 461 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEVDHHHHHH

SEQ ID NO: 229 - RNA, 112 nt, synthetic construct
  misc_feature 1..112: note = Aptamer
  source 1..112: mol_type = other RNA
gggtgcatcgtttacgcgcaacataaaaatttaaagtgctcagttgtcaatctatgactgctgctgaggaaggatatgagaaacaaacaaacgtatggcggtctccaacagg

SEQ ID NO: 230 - DNA, 20 bp, synthetic construct
  misc_feature 1..20: note = Antisense Oligo
  source 1..20: mol_type = other DNA
cctgttggagaccgccatac

SEQ ID NO: 231 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 232 - protein, 456 aa, Homo sapiens
QEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSH

SEQ ID NO: 233 - protein, 924 aa, Homo sapiens
MPGPSPGLRRALLGLWAALGLGLFGLSAVSQEPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLVCNVTLGGENRETRENVTIYSFPAPLLTLSEPSVSEGQMVTVTCAAGAQALVTLEGVPAAVPGQPAQLQLNATENDDRRSFFCDATLDVDGETLIKNRSAELRVLYAPRLDDSDCPRSWTWPEGPEQTLRCEARGNPEPSVHCARSDGGAVLALGLLGPVTRALSGTYRCKAANDQGEAVKDVTLTVEYAPALDSVGCPERITWLEGTEASLSCVAHGVPPPDVICVRSGELGAVIEGLLRVAREHAGTYRCEATNPRGSAAKNVAVTVEYGPRFEEPSCPSNWTWVEGSGRLFSCEVDGKPQPSVKCVGSGGATEGVLLPLAPPDPSPRAPRIPRVLAPGIYVCNATNRHGSVAKTVVVSAESPPEMDESTCPSHQTWLEGAEASALACAARGRPSPGVRCSREGIPWPEQQRVSREDAGTYHCVATNAHGTDSRTVTVGVEYRPVVAELAASPPGGVRPGGNFTLTCRAEAWPPAQISWRAPPGALNIGLSSNNSTLSVAGAMGSHGGEYECAATNAHGRHARRITVRVAGPWLWVAVGGAAGGAALLAAGAGLAFYVQSTACKKGEYNVQEAESSGEAVCLNGAGGGAGGAAGAEGGPEAAGGAAESPAEGEVFAIQLTSA

SEQ ID NO: 234 - protein, 804 aa, Homo sapiens
EPFWADLQPRVAFVERGGSLWLNCSTNCPRPERGGLETSLRRNGTQRGLRWLARQLVDIREPETQPVCFFRCARRTLQARGLIRTFQRPDRVELMPLPPWQPVGENFTLSCRVPGAGPRASLTLTLLRGAQELIRRSFAGEPPRARGAVLTATVLARREDHGANFSCRAELDLRPHGLGLFENSSAPRELRTFSLSPDAPRLAAPRLLEVGSERPVSCTLDGLFPASEARVYLALGDQNLSPDVTLEGDAFVATATATASAEQEGARQLVCNVTLGGENRETRENVTIYSFPAPLLTLSEPSVSEGQMVTVTCAAGAQALVTLEGVPAAVPGQPAQLQLNATENDDRRSFFCDATLDVDGETLIKNRSAELRVLYAPRLDDSDCPRSWTWPEGPEQTLRCEARGNPEPSVHCARSDGGAVLALGLLGPVTRALSGTYRCKAANDQGEAVKDVTLTVEYAPALDSVGCPERITWLEGTEASLSCVAHGVPPPDVICVRSGELGAVIEGLLRVAREHAGTYRCEATNPRGSAAKNVAVTVEYGPRFEEPSCPSNWTWVEGSGRLFSCEVDGKPQPSVKCVGSGGATEGVLLPLAPPDPSPRAPRIPRVLAPGIYVCNATNRHGSVAKTVVVSAESPPEMDESTCPSHQTWLEGAEASALACAARGRPSPGVRCSREGIPWPEQQRVSREDAGTYHCVATNAHGTDSRTVTVGVEYRPVVAELAASPPGGVRPGGNFTLTCRAEAWPPAQISWRAPPGALNIGLSSNNSTLSVAGAMGSHGGEYECAATNAHGRHARRITVRVAGPW

SEQ ID NO: 235 - RNA, 12 nt, synthetic construct
  misc_feature 1..12: note = Synthetic Spacer Sequence
  source 1..12: mol_type = other RNA
aaacaaacaaac

SEQ ID NO: 236 - RNA, 20 nt, synthetic construct
  misc_feature 1..20: note = Synthetic Sense Binding Sequence
  source 1..20: mol_type = other RNA
gtatggcggtctccaacagg

(19) United States
(12) Patent Application Publication
(10) Pub. No.: US 2024/0209375 A1
(43) Pub. Date: Jun. 27, 2024

(54) Aptamers For Personal Health Care Applications

(71) Applicant: The Procter & Gamble Company, Cincinnati, OH (US)

(72) Inventors: Juan Esteban Velasquez, Cincinnati, OH (US); Spencer Christopher Rupard, Morrow, OH (US); Amy Violet Trejo, Mason, OH (US); Adam Michael Pitz, Morrow, OH (US); Kelly Lee Schmeichel, Cincinnati, OH (US); Erin Nicole Swigart, Morrow, OH (US); Gregory Allen Penner, London (CA)

(21) Appl. No.: 18/512,207

(22) Filed: Nov. 17, 2023

Related U.S. Application Data
(63) Continuation of application No. 17/356,828, filed on Jun. 24, 2021, now Pat. No. 11,859,187.
(60) Provisional application No. 63/043,952, filed on Jun. 25, 2020.

(51) Int. Cl. C12N 15/113; A61K 31/7088; A61K 47/54; A61K 47/60

(52) U.S. Cl. CPC C12N 15/1138; A61K 31/7088; A61K 47/549; A61K 47/60; C12N 2310/16; C12N 2310/351; C12N 2320/31; C12N 2320/32

(57) ABSTRACT

An aptamer composition is disclosed which has one or more oligonucleotides that include at least one of deoxyribonucleotides, ribonucleotides, derivatives of deoxyribonucleotides, derivatives of ribonucleotides, or mixtures thereof. The aptamer composition has a binding affinity for one or more cellular membrane glycoproteins selected from the group consisting of: intercellular adhesion molecule 1 (ICAM-1), low-density lipoprotein receptor (LDLR) family members, and cadherin-related family member 3 (CDHR3), preferably intercellular adhesion molecule 1 (ICAM-1), and is configured to reduce the binding of one or more human rhinoviruses to the intercellular adhesion molecule 1 (ICAM-1).

### FIELD OF THE INVENTION

Described herein are nucleic acid aptamers that have a high binding affinity and specificity for cellular membrane glycoproteins and preferably for intercellular adhesion molecule-1 (“ICAM-1”), and more particularly the use of such aptamers to inhibit human rhinovirus binding to such glycoproteins and entering into cells within the nasal cavity and throat.

### INCORPORATION BY REFERENCE OF THE SEQUENCE LISTING

This application contains, as a separate part of disclosure, a Sequence Listing in computer-readable form (Filename: 15819MC_ST26.xml; Size 304,712 bytes; Created: Jan. 11, 2024) which is incorporated by reference herein in its entirety.

### BACKGROUND OF THE INVENTION

Aptamers are short single-stranded oligonucleotides, with a specific and complex three-dimensional shape, that bind to target molecules. The molecular recognition of aptamers is based on structure compatibility and intermolecular interactions, including electrostatic forces, van der Waals interactions, hydrogen bonding, and π-π stacking interactions of aromatic rings with the target material. The targets of aptamers include, but are not limited to, peptides, proteins, nucleotides, amino acids, antibiotics, low molecular weight organic or inorganic compounds, and even whole cells. The dissociation constant of aptamers typically varies between micromolar and picomolar levels, which is comparable to the affinity of antibodies to their antigens. Aptamers can also be designed to have high specificity, enabling the discrimination of target molecules from closely related derivatives.

Aptamers are usually designed in vitro from large libraries of random nucleic acids by Systematic Evolution of Ligands by Exponential Enrichment (SELEX). The SELEX method is first introduced in 1990 when single stranded RNAs are selected against low molecular weight dyes (Ellington, A. D., Szostak, J. W., 1990. Nature 346: 818-822). A few years later, single stranded DNA aptamers and aptamers containing chemically modified nucleotides are also described (Ellington, A. D., Szostak, J. W., 1992. Nature 355: 850-852; Green, L. S., et al., 1995. Chem. Biol. 2: 683-695). Since then, aptamers for hundreds of microscopic targets, such as cations, small molecules, proteins, cells, or tissues, have been selected. A compilation of examples from the literature is included in the database at the website: http://www.aptagen.com/aptamer-index/aptamer-list.aspx.

The common cold is the most frequent illness in the U.S., with 62 million people being infected each year. Adults can be infected with a common cold 2-4 times per year, while children can be infected 8-12 times per year. This leads to morbidity, frequent absences from school and work, reduced productivity, and inappropriate use of antibiotics. This translates into costing the U.S. $60 billion annually.

Human rhinoviruses cause 50-80% of common colds. Rhinoviruses are small (30 nm), nonenveloped single-stranded RNA viruses. Although rhinovirus infections are mild and self-limiting in immunocompetent hosts, it is associated with pneumonia in immunosuppressed patients, bronchiolitis in infants, and can exacerbate pre-existing pulmonary diseases such as asthma and chronic obstructive pulmonary disease.

Rhinovirus infection predominately occurs in the nasopharynx when the virus attaches to surface receptors on the nasal epithelium and infects the host cells. Ninety percent of rhinoviruses attach to ICAM-1 receptors that line the airways. Once the virus enters into the cell, it hijacks the cell's replication machinery to make copies of itself. This results in cell lysis and death, allowing the virus progeny to spread to other nearby cells to repeat the infectious cycle. Ultimately, this triggers a host immune response leading to respiratory symptoms (e.g. cough, rhinorrhea, congestion, sore throat, etc.). Despite the enormous public health burden, there are no licensed vaccines or antiviral drugs for human rhinovirus.

Aptamers against target proteins such as intercellular adhesion molecule 1 (ICAM-1) have previously been described. However, no data for the binding of such aptamers to the membrane bound protein or the capacity of these aptamers to prevent the binding of natural ligands or human rhinoviruses to ICAM-1 have been reported. Thus, a need still exists for aptamers that selectively bind to cellular membrane glycoproteins, including ICAM-1, and that prevent the binding of human rhinoviruses to such glycoproteins, mitigating symptoms for common cold or preventing (re)infection.

### SUMMARY OF THE INVENTION

Described herein is the use of SELEX for the selection of aptamers against the intercellular adhesion molecule 1 (ICAM-1) and the use of such aptamers for the prevention of binding of human rhinoviruses to such glycoprotein.

Described herein is also an aptamer composition. The aptamer composition comprises at least one oligonucleotide consisting of: deoxyribonucleotides, ribonucleotides, derivatives of deoxyribonucleotides, derivatives of ribonucleotides, and mixtures thereof; wherein said aptamer composition has a binding affinity for intercellular adhesion molecule 1 (ICAM-1), wherein the aptamer composition can reduce the binding of one or more human rhinoviruses to said intercellular adhesion molecule 1 (ICAM-1) and wherein the aptamer composition comprises
(a) at least one oligonucleotide selected from the group consisting of oligonucleotides with at least 80% nucleotide sequence identity to sequences selected from the group consisting of SEQ ID NO: 1 to SEQ ID NO: 200; and/or;
(b) at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

The aptamer composition may further show a binding affinity for one or more of low-density lipoprotein receptor (LDLR) family members, cadherin-related family member 3 (CDHR3), and combinations thereof.

The aptamer composition may comprise at least one oligonucleotide selected from the group consisting of SEQ ID NO: 2, SEQ ID NO: 4, SEQ ID NO: 5, SEQ ID NO: 7, and SEQ ID NO: 8. Also described herein is a personal health care composition. The personal health care composition comprises the aptamer composition as described herein. The personal health care composition may comprise at least one nucleic acid aptamer; wherein the nucleic acid aptamer has a binding affinity for intercellular adhesion molecule 1 (ICAM-1), wherein the nucleic acid aptamer reduces the binding of one or more human rhinoviruses to the intercellular adhesion molecule 1 (ICAM-1) and wherein the aptamer composition comprises
(a) at least one oligonucleotide selected from the group consisting of oligonucleotides with at least 80% nucleotide sequence identity to sequences selected from the group consisting of SEQ ID NO: 1 to SEQ ID NO: 200; and/or;
(b) at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ TD NO: 210, SEQ ID NO: 211, and SEQ ID NO: 212.

The aptamer composition may further show a binding affinity for one or more of low-density lipoprotein receptor (LDLR) family members, cadherin-related family member 3 (CDHR3), and combinations thereof.

A method for delivering a personal health care composition to the upper respiratory tract is also provided. The method comprises administering a personal health care composition as described herein; the personal health care composition comprises at least one nucleic acid aptamer; wherein the at least one nucleic acid aptamer has a binding affinity for intercellular adhesion molecule 1 (ICAM-1), wherein the nucleic acid aptamer reduces the binding of one or more human rhinoviruses to the intercellular adhesion molecule 1 (ICAM-1) and wherein the aptamer composition comprises
(a) at least one oligonucleotide selected from the group consisting of oligonucleotides with at least 80% nucleotide sequence identity to sequences selected from the group consisting of SEQ ID NO: 1 to SEQ ID NO: 200; and/or;
(b) at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

In one aspect, the personal health care composition can also comprise one or more active ingredients; wherein the at least one nucleic acid aptamer and the one or more active ingredients are covalently or non-covalently attached.

Described herein is further the use of the aptamer composition as disclosed herein and/or the use of the personal health care composition as disclosed herein for inhibiting human rhinovirus infection by inhibiting binding to the intercellular adhesion molecule 1 (ICAM-1) and thereby inhibiting entering into cells within the nasal cavity and throat. The use may include delivering the aptamer composition and/or the personal health care composition as disclosed herein to the upper respiratory tract.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 illustrates a schematic of the DNA library.

FIG. 2 illustrates the aptamer selection strategy during selection rounds 1 to 11.

FIG. 3 shows a schematic of the aptamer splits selection strategy during selection rounds 12 to 14.

FIG. 4 illustrates the enrichment trajectories for the top twenty aptamers.

FIG. 5 illustrates the binding assay results of selected aptamers on HNepC and HEK293 cells.

FIGS. 6A-6D show the fluorescently labelled aptamer Nas.R-4 bound to HNepC and to HEK293 cells. FIG. 6A shows the fluorescence image and FIG. 6B shows the brightfield image of the HNepC cells. FIG. 6C shows the fluorescence image and FIG. 6D shows the brightfield image of the HEK293 cells.

FIGS. 7A-7H show the viral inhibition test on HeLa cells using aptamer Nas.R-2, aptamer Nas.R-8 and a negative control aptamer. FIG. 7A shows the fluorescence image and FIG. 7B shows the brightfield image using the Nas.R-2 aptamer; FIG. 7C shows the fluorescence image and FIG. 7D shows the brightfield image using the Nas.R-8 aptamer; FIG. 7E shows the fluorescence image and FIG. 7F shows the brightfield image using the control aptamer; and FIG. 7G shows the fluorescence image and FIG. 7H shows the brightfield image of cells only.

FIG. 8 illustrates the surface plasmon resonance curve of aptamers Nas.R-1, Nas.R-2, Nas.R-4, and Nas.R-8 with 250 nM exogenous ICAM-1.

FIG. 9 illustrates the surface plasmon resonance curve of aptamers Nas.R-1, Nas.R-2, Nas.R-4, and Nas.R-8 with 250 nM human serum albumin.

FIG. 10 shows the amino acid sequence alignment of ICAM-1 (see SEQ ID NO: 214), ICAM-3 (see SEQ ID NO: 232), and ICAM-5 (see SEQ ID NO: 234).

FIG. 11 illustrates examples of sequences that exhibited higher enrichment levels with nasal cells positive selection than with HEK293 cells positive selection.

FIG. 12 illustrates examples of sequences in selection round 14 that exhibited higher enrichment levels with HEK293 positive selection than with positive selection against nasal cells.

FIG. 13 shows alignment of exemplary sequences with at least 90% nucleotide sequence identity that are identified during the selection process (Nas-3 (see, e.g., SEQ ID NO: 103) and Nas-88 (see, e.g., SEQ ID NO: 188)).

FIG. 14 shows alignment of exemplary sequences with at least 70% nucleotide sequence identity that are identified during the selection process (Nas-45 (see, e.g., SEQ ID NO: 145), Nas-8 (see, e.g., SEQ ID NO: 108), Nas-47 (see, e.g., SEQ ID NO: 147), and Nas-78 (see, e.g., SEQ ID NO: 178).

FIG. 15 shows alignment of exemplary sequences with at least 50% nucleotide sequence identity that are identified during the selection process (Nas-13 (see, e.g., SEQ ID NO: 113), Nas-97 (see, e.g., SEQ ID NO: 197), Nas-31 (see, e.g., SEQ ID NO: 131), Nas-93 (see, e.g., SEQ ID NO: 193), Nas-39 (see, e.g., SEQ ID NO: 139), Nas-82 (see, e.g., SEQ ID NO: 182), Nas-61 (see, e.g., SEQ ID NO: 161), Nas-91 (see, e.g., SEQ ID NO: 191), Nas-87 (see, e.g., SEQ ID NO: 187), and Nas-94 (see, e.g., SEQ ID NO: 194).

FIG. 16 illustrates the results of the motif analysis of random region of aptamer Nas.R-1.

FIG. 17 illustrates the predicted secondary structures of aptamer Nas.R-1 and its conserved motifs (see, e.g., SEQ ID NO: 201, and SEQ ID NO: 202).

FIG. 18 illustrates the results of the motif analysis of random region of aptamer Nas.R-4.

FIG. 19 illustrates the results of the motif analysis of random region of aptamer Nas.R-8.

FIG. 20 illustrates the motif analysis of the random region of the top 100 aptamers shown as DNA sequences.

### DETAILED DESCRIPTION OF THE INVENTION

### I. Definitions

As used herein, the term “aptamer” refers to a single stranded oligonucleotide or a peptide that has a binding affinity for a specific target.

As used herein, the term “nucleic acid” refers to a polymer or oligomer of nucleotides. Nucleic acids are also referred as “ribonucleic acids” when the sugar moiety of the nucleotides is D-ribose and as “deoxyribonucleic acids” when the sugar moiety is 2-deoxy-D-ribose.

As used herein, the term “nucleotide” refers to a compound consisting of a nucleoside esterified to a monophosphate, polyphosphate, or phosphate-derivative group via the hydroxyl group of the 5-carbon of the sugar moiety. Nucleotides are also referred as “ribonucleotides” when the sugar moiety is D-ribose and as “deoxyribonucleotides” when the sugar moiety is 2-deoxy-D-ribose.

As used herein, the term “nucleoside” refers to a glycosylamine consisting of a nucleobase, such as a purine or pyrimidine, usually linked to a 5-carbon sugar (e.g. D-ribose or 2-deoxy-D-ribose) via a β-glycosidic linkage. Nucleosides are also referred as “ribonucleosides” when the sugar moiety is D-ribose and as “deoxyribonucleosides” when the sugar moiety is 2-deoxy-D-ribose.

As used herein, the term “nucleobase” refers to a compound containing a nitrogen atom that has the chemical properties of a base. Non-limiting examples of nucleobases are compounds comprising pyridine, purine, or pyrimidine moieties, including but not limited to, adenine, guanine, hypoxanthine, thymine, cytosine, and uracil.

As used herein, the term “oligonucleotide” refers to an oligomer composed of nucleotides.

As used herein, the term “identical” or “sequence identity”, in the context of two or more oligonucleotides, nucleic acids, or aptamers, refers to two or more sequences that are the same or have a specified percentage of nucleotides that are the same, when compared and aligned for maximum correspondence, as measured using sequence comparison algorithms or by visual inspection.

As used herein, the term “substantially homologous” or “substantially identical”, in the context of two or more oligonucleotides, nucleic acids, or aptamers, generally refers to two or more sequences or subsequences that have at least 40%, 60%, 80%, 90%, 95%, 96%, 97%, 98% or 99% nucleotide identity, when compared and aligned for maximum correspondence, as measured using sequence comparison algorithms or by visual inspection.

As used herein, the term “epitope” refers to the region of a target that interacts with the aptamer. An epitope can be a contiguous stretch within the target or can be represented by multiple points that are physically proximal in a folded form of the target.

As used herein, the term “motif” refers to the sequence of contiguous, or series of contiguous, nucleotides occurring in a library of aptamers with binding affinity towards a specific target and that exhibits a statistically significant higher probability of occurrence than would be expected compared to a library of random oligonucleotides. The motif sequence is frequently the result or driver of the aptamer selection process.

As used herein, the term “personal health care compositions” refers to compositions in a form that is directly deliverable to the upper respiratory tract.

As used herein, “a pharmaceutically effective amount” refers to an amount sufficient to confer a therapeutic effect on the subject. In some aspects the therapeutic effect is reduced rhinovirus binding to cellular membrane glycoproteins such as ICAM-1, reduced severity and/or duration of a cold, or reduced incidence of respiratory illness due to rhinovirus.

### II. Aptamer Composition

The human rhinoviruses (RV) are the predominant cause of the common cold. They are classified in three groups (RV-A, RV-B, and RV-C), including around 160 types that express different surface proteins. Despite this diversity, rhinoviruses utilize mostly three glycoproteins of epithelial cells to cross the cellular membrane and access the host cell replication machinery: intercellular adhesion molecule 1 or ICAM-1 protein, utilized by the majority of RV-A and all RV-B types; low-density lipoprotein receptor or LDLR family members, utilized by at least twelve RV-A types; and cadherin-related family member 3 or CADHR3 proteins, utilized mostly by RV-C types.

An aptamer composition may comprise at least one oligonucleotide selected from the group consisting of deoxyribonucleotides, ribonucleotides, derivatives of deoxyribonucleotides, derivatives of ribonucleotides, and mixtures thereof, wherein the aptamer composition has a binding affinity for intercellular adhesion molecule 1 (ICAM-1). In one aspect, the aptamer composition may have a binding affinity for one or more cellular membrane glycoproteins selected from the group consisting of intercellular adhesion molecule 1 (ICAM-1), low-density lipoprotein receptor (LDLR) family members, and cadherin-related family member 3 (CDHR3) and combinations thereof. Preferably the one or more cellular membrane glycoprotein is intercellular adhesion molecule 1 (ICAM-1). The aptamer composition can reduce the binding of one or more human rhinoviruses to the intercellular adhesion molecule 1 (ICAM-1).

The aptamer composition may comprise at least one oligonucleotide selected from the group consisting of oligonucleotides with at least 80% nucleotide sequence identity to sequences selected from the group consisting of SEQ ID NO: 1 to SEQ ID NO: 200. The aptamer composition may comprise at least one oligonucleotide selected from the group consisting of oligonucleotides with at least 90% nucleotide sequence identity to sequences selected from the group consisting of SEQ ID NO: 1 to SEQ ID NO: 200. The aptamer composition may comprise at least one oligonucleotide selected from the group consisting of oligonucleotides with at least 95% nucleotide sequence identity to sequences selected from the group consisting of SEQ ID NO: 1 to SEQ ID NO: 200. The aptamer composition may comprise at least one oligonucleotide selected from the group consisting of SEQ ID NO: 1 to SEQ ID NO: 200. A non-limiting example of oligonucleotide with at least 90% nucleotide sequence identity to SEQ ID NO: 3 is SEQ ID NO: 88.

The aptamer composition may comprise at least one oligonucleotide selected from the group consisting of oligonucleotides containing at least 10 contiguous nucleotides from sequences selected from the group consisting of SEQ ID NO: 201 to SEQ ID NO: 212.

The aptamer composition may comprise at least one oligonucleotide selected from the group consisting of SEQ ID NO: 2, SEQ ID NO: 4, SEQ ID NO: 5, SEQ ID NO: 7, and SEQ ID NO: 8. The aptamer composition may comprise at least one oligonucleotide selected from the group consisting of oligonucleotides with at least 50% nucleotide sequence identity to sequences selected from the group consisting of SEQ ID NO: 2, SEQ ID NO: 4, SEQ ID NO: 5, SEQ ID NO: 7, and SEQ ID NO: 8. The aptamer composition may comprise at least one oligonucleotide selected from the group consisting of oligonucleotides with at least 70% nucleotide sequence identity to sequences selected from the group consisting of SEQ ID NO: 2, SEQ ID NO: 4, SEQ ID NO: 5, SEQ ID NO: 7, and SEQ ID NO: 8. The aptamer composition may comprise at least one oligonucleotide selected from the group consisting of oligonucleotides with at least 90% nucleotide sequence identity to sequences selected from the group consisting of SEQ ID NO: 2, SEQ ID NO: 4, SEQ ID NO: 5, SEQ ID NO: 7, and SEQ ID NO: 8. A non-limiting example of oligonucleotide with at least 50% nucleotide sequence identity to SEQ ID NO: 4 is SEQ ID NO: 35. Non-limiting examples of oligonucleotides with at least 50% nucleotide sequence identity to SEQ ID NO: 7 are SEQ ID NO: 36, SEQ ID NO: 50, SEQ ID NO: 77, and SEQ ID NO: 97. Non-limiting examples of oligonucleotides with at least 50% nucleotide sequence identity to SEQ ID NO: 8 are SEQ ID NO: 12, SEQ ID NO: 22, SEQ ID NO: 29, SEQ ID NO: 44, SEQ ID NO: 45, SEQ ID NO: 53, SEQ ID NO: 63, SEQ ID NO: 74, and SEQ ID NO: 89.

The at least one oligonucleotide can comprise one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212. The aptamer composition may comprise at least one oligonucleotide comprising a sequence of nucleotides with at least 80% nucleotide sequence identity to sequences selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212. The aptamer composition may comprise at least one oligonucleotide comprising a sequence of nucleotides with at least 90% nucleotide sequence identity to sequences selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212. The aptamer composition may comprise at least one oligonucleotide comprising a sequence of nucleotides with at least 95% nucleotide sequence identity to sequences selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

In one aspect, the aptamer composition has a binding affinity for the human intercellular adhesion molecule 1 (ICAM-1) (SEQ ID NO: 213), its natural variants, polymorphic variants, or any post-translationally modified versions of said protein. Non-limiting examples of posttranslational modifications of ICAM-1 are disulfide bonds (e.g. between Cys48 and Cys92, Cys52 and Cys96, Cys135 and Cys186, Cys237 and Cys290, Cys332 and Cys371, Cys403 and Cys419, Cys431 and Cys457), glycosylations (e.g. at Asn130, Asn145, Asn183, Asn202, Asn267, Asn296, Asn385, and Asn406), phosphorylations (e.g. at Thr521 or Thr530), and ubiquitination.

In one aspect, the aptamer composition has a binding affinity for the extracellular domain of human intercellular adhesion molecule 1 (ICAM-1) (SEQ ID NO: 214) or any post-translationally modified versions of said domain. In one aspect, the aptamer composition has a binding affinity for one or more domains of the intercellular adhesion molecule 1 (ICAM-1) selected from the group consisting of: Ig-like C2-type 1 domain (SEQ ID NO: 215), Ig-like C2-type 2 domain (SEQ ID NO: 216), Ig-like C2-type 3 domain (SEQ ID NO: 217), Ig-like C2-type 4 domain (SEQ ID NO: 218), Ig-like C2-type 5 domain (SEQ ID NO: 219), any post-translationally modified versions of said domains, and mixtures thereof. In one aspect, the aptamer composition has a binding affinity for the Ig-like C2-type 1 domain (SEQ ID NO: 215) of the intercellular adhesion molecule 1 (ICAM-1), any post-translationally modified versions of said domain, and mixtures thereof.

In one aspect, the aptamer composition has a binding affinity for one or more regions of the human intercellular adhesion molecule 1, wherein said regions comprise an amino acid sequence selected from the group consisting of SEQ ID NO: 220, SEQ ID NO: 221, SEQ ID NO: 222, SEQ ID NO: 223, and fragments of said sequences.

Chemical modifications can introduce new features into the aptamers such as different molecular interactions with the target, improved binding capabilities, enhanced stability of oligonucleotide conformations, or increased resistance to nucleases. In one aspect, the at least one oligonucleotide of the aptamer composition may comprise natural or non-natural nucleobases. Natural nucleobases are adenine, cytosine, guanine, thymine, and uracil. Non-limiting examples of non-natural nucleobases can include hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, bromouracil, 5-iodouracil, and mixtures thereof.

Modifications of the phosphate backbone of the oligonucleotides can also increase the resistance against nuclease digestion. In one aspect, the nucleosides of the oligonucleotides may be linked by a chemical motif selected from the group consisting of natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, fluorophosphate, and mixtures thereof. In one aspect, the nucleosides of the oligonucleotides may be linked by natural phosphate diesters.

In one aspect, the sugar moiety of the nucleosides of the oligonucleotides may be selected from the group consisting of ribose, deoxyribose, 2′-fluoro deoxyribose, 2′-O-methyl ribose, 2′-O-(3-amino)propyl ribose, 2′-O-(2-methoxy)ethyl ribose, 2′-O-2-(N,N-dimethylaminooxy)ethyl ribose, 2′-O-2-[2-(N,N-dimethylamino)ethyloxy]ethyl ribose, 2′-O—N,N-dimethylacetamidyl ribose, N-morpholinophosphordiamidate, α-deoxyribofuranosyl, other pentoses, hexoses, and mixtures thereof.

In one aspect, the derivatives of ribonucleotides or said derivatives of deoxyribonucleotides may be selected from the group consisting of locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.

In one aspect, the nucleotides at the 5′- and 3′-ends of the at least one oligonucleotide may be inverted. In one aspect, at least one nucleotide of the at least one oligonucleotide may be fluorinated at the 2′ position of the pentose group. In one aspect, the pyrimidine nucleotides of said at least one oligonucleotide may be fluorinated at the 2′ position of the pentose group. In one aspect, said aptamer composition may comprise at least one polymeric material, wherein said at least one polymeric material is covalently linked to said at least one oligonucleotide. In one aspect, said at least one polymeric material may be polyethylene glycol.

In one aspect, said at least one oligonucleotide may be between about 10 and about 200 nucleotides in length. In one aspect, said at least one oligonucleotide may be less than about 100 nucleotides in length, alternatively said at least one oligonucleotide may be less than about 50 nucleotides in length.

In one aspect, said at least one oligonucleotide may be covalently or non-covalently attached to one or more active ingredients. In one aspect, said one or more active ingredients may be selected from the group comprising respiratory illness treatment agents, cold-treatment agents, flu-treatment agents, antiviral agents, antimicrobial agents, cooling sensates, warming sensates, malodor absorbing agents, natural extracts, peptides, enzymes, pharmaceutical active ingredients, metal compounds, and mixtures thereof. In one aspect, said one or more active ingredients can include, but are not limited to, pharmaceutical active ingredients, menthol, levomenthol, zinc and salts thereof, eucalyptus, camphor, and combinations thereof. Suitable active ingredients include any material that is generally considered as safe and that provides health care benefits.

In one aspect, said at least one oligonucleotide may be non-covalently attached to said one or more active ingredients via molecular interactions. Examples of molecular interactions are electrostatic forces, van der Waals interactions, hydrogen bonding, and π-π stacking interactions of aromatic rings.

In one aspect, said at least one oligonucleotide may be covalently attached to said one or more active ingredients using one or more linkers or spacers. Non-limiting examples of linkers are chemically labile linkers, enzyme-labile linkers, and non-cleavable linkers. Examples of chemically labile linkers are acid-cleavable linkers and disulfide linkers. Acid-cleavable linkers take advantage of low pH to trigger hydrolysis of an acid-cleavable bond, such as a hydrazone bond, to release the active ingredient or payload. Disulfide linkers can release the active ingredients under reducing environments. Examples of enzyme-labile linkers are peptide linkers that can be cleaved in the presence of proteases and β-glucuronide linkers that are cleaved by glucuronidases releasing the payload. Non-cleavable linkers can also release the active ingredient if the aptamer is degraded by nucleases.

In one aspect, said at least one oligonucleotide may be covalently or non-covalently attached to one or more nanomaterials. In the present invention, said at least one oligonucleotide and said one or more active ingredients may be covalently or non-covalently attached to one or more nanomaterials. In one aspect, said one or more active ingredients may be carried by said one or more nanomaterials. Non-limiting examples of nanomaterials can include gold nanoparticles, nano-scale iron oxides, carbon nanomaterials (such as single-walled carbon nanotubes and graphene oxide), mesoporous silica nanoparticles, quantum dots, liposomes, poly (lactide-co-glycolic acids) nanoparticles, polymeric micelles, dendrimers, serum albumin nanoparticles, DNA-based nanomaterials, and combinations thereof. These nanomaterials can serve as carriers for large volumes of active ingredients, while the aptamers can facilitate the delivery of the nanomaterials with the actives to the expected target.

Nanomaterials can have a variety of shapes or morphologies. Non-limiting examples of shapes or morphologies can include spheres, rectangles, polygons, disks, toroids, cones, pyramids, rods/cylinders, and fibers. In the context of the present invention, nanomaterials usually have at least one spatial dimension that is less than about 100 μm and more preferably less than about 10 μm. Nanomaterials comprise materials in solid phase, semi-solid phase, or liquid phase.
1. Aptamers can also be peptides that bind to targets with high affinity and specificity. These peptide aptamers can be part of a scaffold protein. Peptide aptamers can be isolated from combinatorial libraries and improved by directed mutation or rounds of variable region mutagenesis and selection. In one aspect, said aptamer composition may comprise at least one peptide or protein; wherein said aptamer composition has a binding affinity for one or more cellular membrane glycoproteins, wherein said one or more cellular membrane glycoproteins can be selected from the group consisting of: intercellular adhesion molecule 1 (ICAM-1), low-density lipoprotein receptor (LDLR) family members, and cadherin-related family member 3 (CDHR3); preferably intercellular adhesion molecule 1 (ICAM-1) and wherein said aptamer is configured to reduce the binding of one or more human rhinoviruses to said cellular membrane glycoproteins, preferably the intercellular adhesion molecule 1 (ICAM-1). In particular said aptamer composition may comprise at least one peptide or protein translated from
   (a) at least one oligonucleotide selected from the group consisting of oligonucleotides with at least 80% nucleotide sequence identity to sequences selected from the group consisting of SEQ ID NO: 1 to SEQ ID NO: 200; and/or;
   (b) at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

### III. Methods of Designing Aptamer Compositions

The method of designing nucleic acid aptamers known as Systematic Evolution of Ligands by Exponential Enrichment (SELEX) has been broadly studied and improved for the selection of aptamers against small molecules and proteins (WO 91/19813). In brief, in the conventional version of SELEX, the process starts with the synthesis of a large library of oligonucleotides consisting of randomly generated sequences of fixed length flanked by constant 5′- and 3′-ends that serve as primers. The oligonucleotides in the library are then exposed to the target ligand and those that do not bind the target are removed. The bound sequences are eluted and amplified by PCR (polymerase chain reaction) to prepare for subsequent rounds of selection in which the stringency of the elution conditions is usually increased to identify the tightest-binding oligonucleotides. In addition to conventional SELEX, there are improved versions such as capillary electrophoresis-SELEX, magnetic bead-based SELEX, cell-SELEX, automated SELEX, complex-target SELEX, among others. A review of aptamer screening methods is found in (1) Kim, Y. S. and M. B. Gu, “Advances in Aptamer Screening and Small Molecule Aptasensors”, Adv. Biochem. Eng. Biotechnol., 2014 140:29-67 (Biosensors based on Aptamers and Enzymes) and (2) Stoltenburg, R., et al. (2007) “SELEX-A (r)evolutionary method to generate high-affinity nucleic acid ligands” Biomol. Eng. 2007 24(4): 381-403, the contents of which are incorporated herein by reference. Although the SELEX method has been broadly applied, it is neither predictive nor standardized for every target. Instead, a method must be developed for each particular target in order for the method to lead to viable aptamers.

Despite the large number of selected aptamers, SELEX has not been routinely applied for the selection of aptamers with binding affinities towards cellular membrane glycoproteins such as intercellular adhesion molecule 1 (ICAM-1), low-density lipoprotein receptor (LDLR) family members, and cadherin-related family member 3 (CDHR3) and that prevent the binding of human rhinoviruses to such proteins. Unexpectedly, the inventors have found that SELEX can be used for the design of aptamers that prevent the binding of human rhinoviruses to the ICAM-1 receptor.

### Selection Library

In SELEX, the initial candidate library is generally a mixture of chemically synthesized DNA oligonucleotides, each comprising a long variable region of n nucleotides flanked at the 3′ and 5′ ends by conserved regions or primer recognition regions for all the candidates of the library. These primer recognition regions allow the central variable region to be manipulated during SELEX in particular by means of PCR.

The length of the variable region determines the diversity of the library, which is equal to 4ⁿ since each position can be occupied by one of four nucleotides A, T, G or C. For long variable regions, huge library complexities arise. For instance, when n=50, the theoretical diversity is 4⁵⁰ or 10³⁰, which is an inaccessible value in practice as it corresponds to more than 105 tons of material for a library wherein each sequence is represented once. The experimental limit is around 10¹⁵ different sequences, which is that of a library wherein all candidates having a variable region of 25 nucleotides are represented. If one chooses to manipulate a library comprising a 30-nucleotide variable region whose theoretical diversity is about 10¹⁸, only 1/1000 of the possibilities will thus be explored. In practice, that is generally sufficient to obtain aptamers having the desired properties. Additionally, since the polymerases used are unreliable and introduce errors at a rate on the order of 10⁻⁴, they contribute to significantly enrich the diversity of the sequence pool throughout the SELEX process. One candidate in 100 will be modified in each amplification cycle for a library with a random region of 100 nucleotides in length, thus leading to the appearance of 10¹³ new candidates for the overall library.

In one aspect, the starting mixture of oligonucleotides may comprise more than about 10⁶ different oligonucleotides and more preferably between about 10¹³ to about 10¹⁵ different oligonucleotides. In one aspect, the length of the variable region may be between about 10 and about 100 nucleotides. In one aspect, the length of the variable region may be between about 20 and about 60 nucleotides. In one aspect, the length of the variable region may be about 40 nucleotides. Random regions shorter than 10 nucleotides may be used but may be constrained in their ability to form secondary or tertiary structures and in their ability to bind to target molecules. Random regions longer than 100 nucleotides may also be used but may present difficulties in terms of cost of synthesis. The randomness of the variable region is not a constraint of the present invention. For instance, if previous knowledge exists regarding oligonucleotides that bind to a given target, libraries spiked with such sequences may work as well or better than completely random ones.

In the design of primer recognition sequences, care should be taken to minimize potential annealing among sequences, fold back regions within sequences, or annealing of the same sequence itself. In one aspect, the length of primer recognition sequences may be between about 10 and about 40 nucleotides. In one aspect, the length of primer recognition sequences may be between about 12 and about 30 nucleotides. In one aspect, the length of primer recognition sequences may be between about 18 and about 26 nucleotides, i.e., about 18, 19, 20, 21, 22, 23, 24, 25 or 26 nucleotides. The length and sequence of the primer recognition sequences determine their annealing temperature. In one aspect, the primer recognition sequences of said oligonucleotides may have an annealing temperature between about 60° C. and about 72° C.

Aptamers can be ribonucleotides (RNA), deoxynucleotides (DNA), or their derivatives. When aptamers are ribonucleotides, the first SELEX step may consist of transcribing the initial mixture of chemically synthesized DNA oligonucleotides via the primer recognition sequence at the 5′ end. After selection, the candidates are converted back into DNA by reverse transcription before being amplified. RNA and DNA aptamers having comparable characteristics have been selected against the same target and reported in the art. Additionally, both types of aptamers can be competitive inhibitors of one another, suggesting potential overlapping of interaction sites.

New functionalities, such as hydrophobicity or photoreactivity, can be incorporated into the oligonucleotides by modifications of the nucleobases before or after selection. Modifications at the C-5 position of pyrimidines or at the C-8 or N-7 positions of purines are especially common and compatible with certain enzymes used during the amplification step in SELEX. In one aspect, said oligonucleotides may comprise natural or non-natural nucleobases. Natural nucleobases are adenine, cytosine, guanine, thymine, and uracil. Non-limiting examples of non-natural nucleobases are hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, 5-bromouracil, 5-iodouracil, and mixtures thereof. Some non-natural nucleobases, such as 5-bromouracil or 5-iodouracil, can be used to generate photo-crosslinkable aptamers, which can be activated by UV light to form a covalent link with the target.

In one aspect, the nucleosides of said oligonucleotides may be linked by a chemical motif selected from the group comprising natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, fluorophosphate, and mixtures thereof. In one aspect, the nucleosides of said oligonucleotides may be linked by natural phosphate diesters.

In one aspect, the sugar moiety of the nucleosides of said oligonucleotides may be selected from the group comprising ribose, deoxyribose, 2′-fluoro deoxyribose, 2′-O-methyl ribose, 2′-O-(3-amino)propyl ribose, 2′-O-(2-methoxy)ethyl ribose, 2′-O-2-(N,N-dimethylaminooxy)ethyl ribose, 2′-O-2-[2-(N,N-dimethylamino)ethyloxy]ethyl ribose, 2′-O—N,N-dimethylacetamidyl ribose, N-morpholinophosphordiamidate, α-deoxyribofuranosyl, other pentoses, hexoses, and mixtures thereof.

In one aspect, said derivatives of ribonucleotides or said derivatives of deoxyribonucleotides may be selected from the group comprising locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.

When using modified nucleotides during the SELEX process, they should be compatible with the enzymes used during the amplification step. Non-limiting examples of modifications that are compatible with commercial enzymes include modifications at the 2′ position of the sugar in RNA libraries. The ribose 2′-OH group of pyrimidine nucleotides can be replaced with 2′-amino, 2′-fluoro, 2′-methyl, or 2′-O-methyl, which protect the RNA from degradation by nucleases. Additional modifications in the phosphate linker, such as phosphorothionate and boranophosphate, are also compatible with the polymerases and confer resistance to nucleases.

In one aspect, at least one nucleotide of said oligonucleotides may be fluorinated at the 2′ position of the pentose group. In one aspect, the pyrimidine nucleotides of said oligonucleotides may be at least partially fluorinated at the 2′ position of the pentose group. In one aspect, all the pyrimidine nucleotides of said oligonucleotides may be fluorinated at the 2′ position of the pentose group. In one aspect, at least one nucleotide of said oligonucleotides may be aminated at the 2′ position of the pentose group.

Another approach, recently described as two-dimensional SELEX, simultaneously applies in vitro oligonucleotide selection and dynamic combinatorial chemistry (DCC), e.g., a reversible reaction between certain groups of the oligonucleotide (amine groups) and a library of aldehyde compounds. The reaction produces imine oligonucleotides, which are selected on the same principles as for conventional SELEX. It is thus possible to identify for a target hairpin RNA modified aptamers that differ from natural aptamers.

A very different approach relates to the use of optical isomers. Natural oligonucleotides are D-isomers. L-analogs are resistant to nucleases but cannot be synthesized by polymerases. According to the laws of optical isomerism, an L-series aptamer can form with its target (T) a complex having the same characteristics as the complex formed by the D-series isomer and the enantiomer (T′) of the target (T). Consequently, if compound T′ can be chemically synthesized, it can be used to perform the selection of a natural aptamer (D). Once identified, this aptamer can be chemically synthesized in an L-series. This L-aptamer is a ligand of the natural target (T).

### Selection Step

Single stranded oligonucleotides can fold to generate secondary and tertiary structures, resembling the formation of base pairs. The initial sequence library is thus a library of three-dimensional shapes, each corresponding to a distribution of units that can trigger electrostatic interactions, create hydrogen bonds, etc. Selection becomes a question of identifying in the library the shape suited to the target, i.e., the shape allowing the greatest number of interactions and the formation of the most stable aptamer-target complex. For small targets (dyes, antibiotics, etc.) the aptamers identified are characterized by equilibrium dissociation constants in the micromolar range, whereas for protein targets Kd values below 10⁻⁹ M are not rare.

Selection in each round occurs by means of physical separation of oligonucleotides associated with the target from free oligonucleotides. Multiple techniques may be applied (chromatography, filter retention, electrophoresis, etc.). The selection conditions are adjusted (relative concentration of target/candidates, ion concentration, temperature, washing, etc.) so that a target-binding competition occurs between the oligonucleotides. Generally, stringency is increased as the rounds proceed in order to promote the capture of oligonucleotides with the highest affinity. In addition, counter-selections or negative selections are carried out to eliminate oligonucleotides that recognize the support or unwanted targets (e.g., filter, beads, etc.).

The SELEX process for the selection of target-specific aptamers is characterized by repetition of five main steps: (1) binding of oligonucleotides to the target, (2) partition or removal of oligonucleotides with low binding affinity, (3) elution of oligonucleotides with high binding affinity, (4) amplification or replication of oligonucleotides with high binding affinity, and (5) conditioning or preparation of the oligonucleotides for the next cycle. This selection process is designed to identify the oligonucleotides with the greatest affinity and specificity for the target material.

In one aspect, a method of designing an aptamer composition may comprise the step of contacting: a) a mixture of oligonucleotides, b) a selection buffer, and c) a target material comprising one or more cellular membrane glycoproteins selected from the group consisting of: intercellular adhesion molecule 1 (ICAM-1), low-density lipoprotein receptor (LDLR) family members, cadherin-related family member 3 (CDHR3), truncations thereof, and mixtures thereof; preferably intercellular adhesion molecule 1 (ICAM-1) and truncations thereof. In another aspect, the method of designing an aptamer composition may comprise the step of contacting: a) a mixture of oligonucleotides, b) a selection buffer, and c) cells expressing one or more cellular membrane glycoproteins selected from the group consisting of: intercellular adhesion molecule 1 (ICAM-1), low-density lipoprotein receptor (LDLR) family members, cadherin-related family member 3 (CDHR3), truncations thereof, and mixtures thereof; preferably intercellular adhesion molecule 1 (ICAM-1) and truncations thereof. In yet another aspect, the method of designing an aptamer composition may comprise the step of contacting: a) a mixture of oligonucleotides, b) a selection buffer, and c) human nasal epithelial cells expressing one or more cellular membrane glycoproteins selected from the group consisting of: intercellular adhesion molecule 1 (ICAM-1), low-density lipoprotein receptor (LDLR) family members, cadherin-related family member 3 (CDHR3), truncations thereof, and mixtures thereof; preferably intercellular adhesion molecule 1 (ICAM-1) and truncations thereof.

In one aspect, said mixture of oligonucleotides may comprise oligonucleotides selected from the group consisting of deoxyribonucleotides, ribonucleotides, derivatives of deoxyribonucleotides, derivatives of ribonucleotides, and mixtures thereof. Furthermore, said one or more cellular membrane glycoproteins or truncations thereof can be isolated, in mixture with other materials such as proteins or peptides, or part of a cell expressing said glycoproteins.

SELEX cycles are usually repeated several times until oligonucleotides with high binding affinity are identified. The number of cycles depends on multiple variables, including target features and concentration, design of the starting random oligonucleotide library, selection conditions, ratio of target binding sites to oligonucleotides, and the efficiency of the partitioning step. In one aspect, said contacting step may be performed at least 5 times. In one aspect, said contacting step may be performed between 6 and 30 times. In one aspect, said method further may comprise the step of removing the oligonucleotides that do not bind said target material during said contacting step.

Oligonucleotides are oligo-anions, each unit having a charge and hydrogen-bond donor/acceptor sites at a particular pH. Thus, the pH and ionic strength of the selection buffer are important and should represent the conditions of the intended aptamer application. In one aspect, the pH of said selection buffer may be between about 2 and about 9, alternatively between about 5 and about 8.

Cations do not only facilitate the proper folding of the oligonucleotides, but also can provide benefits. In one aspect, said selection buffer may comprise cations. Non-limiting examples of cations are Na⁺, K⁺, Mg²⁺, Ca²⁺.

In order for the aptamers to maintain their structures and function during their application, the in vitro selection process can be carried out under conditions similar to those for which they are being developed. In one aspect, said selection buffer may comprise a solution or suspension of a personal health care composition selected from the group comprising tablets, lyophilized tablets, lollipops, lozenges, liquid center-filled confectioneries, candies, powders, granular substances, films, liquids, solutions, suspensions, mouth rinses or gargles, saline washes, dispersible fluids, sprays, quick dissolving fibers, vapors, creams, ointments, powders, granular substances, films, and combinations thereof.

In one aspect, said selection buffer may comprise at least one surfactant. In one aspect, the at least one surfactant may be selected from the group consisting of anionic surfactants, amphoteric or zwitterionic surfactants, and mixtures thereof. Non-limiting examples of anionic surfactants are alkyl and alkyl ether sulfates or sulfonates, including ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate, and combinations thereof. Non-limiting amphoteric surfactants include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate, including cocoamphoacetate, cocoamphodiacetate, lauroamphoacetate, lauroamphodiacetate, and mixtures thereof. Non-limiting examples of zwitterionic surfactants include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate, and betaine.

The selection buffer may comprise at least one material selected from the group comprising: aqueous carriers, gel matrixes, silicone conditioning agents, organic conditioning materials, non-ionic polymers, deposition aids, rheology modifier/suspending agents, benefit agents, and mixtures thereof. Non-limiting examples of aqueous carriers are water and water solutions of lower alkyl alcohols and polyhydric alcohols, including ethanol, isopropanol, propylene glycol, hexylene glycol, glycerin, and propane diol. Non-limiting examples of gel matrixes include water solutions of fatty alcohols, including cetyl alcohol, stearyl alcohol, behenyl alcohol, and mixtures thereof. Non-limiting examples of silicone conditioning agents include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, sugar or polysaccharide groups, fluorine-modified alkyl groups, alkoxy groups, or combinations of such groups. Non-limiting examples of organic conditioning materials include hydrocarbon oils, polyolefins, fatty esters, fluorinated conditioning compounds, fatty alcohols, alkyl glucosides and alkyl glucoside derivatives, quaternary ammonium compounds, polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M, and mixtures thereof. Non-limiting examples of non-ionic polymers include polyalkylene glycols, such as polyethylene glycols. Non-limiting examples of deposition aids include copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone; vinyl esters, vinyl alcohol (made by hydrolysis of polyvinyl acetate), maleic anhydride, propylene glycol, and ethylene glycol, cationic celluloses, cationic starches, and cationic guar gums. Non-limiting examples of rheology modifier/suspending agents include homopolymers based on acrylic acid, methacrylic acid or other related derivatives, alginic acid-based materials, and cellulose derivatives. Non-limiting examples of benefit agents include brightening agents, strengthening agents, anti-fungal agents, anti-bacterial agents, anti-microbial agents, anti-dandruff agents, anti-malodor agents, perfumes, olfactory enhancement agents, anti-itch agents, cooling agents, anti-adherence agents, moisturization agents, smoothness agents, surface modification agents, antioxidants, natural extracts and essential oils, dyes, pigments, bleaches, nutrients, peptides, vitamins, enzymes, chelants, and mixtures thereof.

Negative selection or counter-selection steps can minimize the enrichment of oligonucleotides that bind to undesired targets or undesired epitopes within a target. In one aspect, said method of designing an aptamer composition may further comprise the step of contacting: a) a mixture of oligonucleotides, b) a selection buffer, and c) one or more undesired targets. Methods for negative selection or counter-selection of aptamers against unbound targets have been published in WO201735666, the content of which is incorporated herein by reference.

The method of designing an aptamer composition may comprise the steps of: a) synthesizing a mixture of oligonucleotides; b) contacting: i. said mixture of oligonucleotides, ii. a selection buffer, and iii. a target material comprising one or more cellular membrane glycoproteins; wherein said glycoproteins are selected from the group consisting of: intercellular adhesion molecule 1 (ICAM-1), its fragments, and combinations thereof, to produce a target suspension; c) removing the liquid phase from said target suspension to produce a target-oligonucleotide mixture; d) contacting said target-oligonucleotide mixture with a washing buffer and removing the liquid phase to produce a target-aptamer mixture; and e) contacting said target-aptamer mixture with an elution buffer and recovering the liquid phase to produce an aptamer mixture. In one aspect, said steps may be performed repetitively at least 5 times. In one aspect, said steps may be performed between 6 and 30 times, preferably less than 20 times.

In another aspect, a method of designing an aptamer composition comprising the steps of: a) synthesizing a random mixture of deoxyribonucleotides comprising oligonucleotides consisting of: i. a T7 promoter sequence at the 5′-end, ii. a variable 40-nucleotide sequence in the middle, and iii. a conserved reverse primer recognition sequence at the 3′end; b) transcribing said random mixture of deoxyribonucleotides using pyrimidine nucleotides fluorinated at the 2′ position of the pentose group and natural purine nucleotides and a mutant T7 polymerase to produce a mixture of fluorinated ribonucleotides; c) contacting: i. said mixture of fluorinated ribonucleotides, ii. a selection buffer, and iii. a target material comprising one or more cellular membrane glycoproteins; wherein said glycoproteins are selected from the group consisting of: intercellular adhesion molecule 1 (ICAM-1), its fragments, and combinations thereof, to produce a target suspension; d) removing the liquid phase from said target suspension to produce a target-oligonucleotide mixture; e) contacting said target-oligonucleotide mixture with a washing buffer and removing the liquid phase to produce a target-aptamer mixture; f) contacting said target-aptamer mixture with an elution buffer and recovering the liquid phase to produce an RNA aptamer mixture; g) reserve transcribing and amplifying said RNA aptamer mixture to produce a DNA copy of said RNA aptamer mixture; and h) sequencing said DNA copy of said RNA aptamer mixture.

### Post-Selection Modification

To enhance stability of the aptamers, chemical modifications can be introduced in the aptamer after the selection process. For instance, the 2′-OH groups of the ribose moieties can be replaced by 2′-fluoro, 2′-amino, or 2′-O-methyl groups. Furthermore, the 3′- and 5′-ends of the aptamers can be capped with different groups, such as streptavidin-biotin, inverted thymidine, amine, phosphate, polyethylene-glycol, cholesterol, fatty acids, proteins, enzymes, fluorophores, among others, making the oligonucleotides resistant to exonucleases or providing some additional benefits. Other modifications are described in previous sections of the present disclosure.

Unlike backbone modifications which can cause aptamer-target interaction properties to be lost, it is possible to conjugate various groups at one of the 3′- or 5′-ends of the oligonucleotide in order to convert it into a delivery vehicle, tool, probe, or sensor without disrupting its characteristics. This versatility constitutes a significant advantage of aptamers, in particular for their application in the current invention. In one aspect, one or more personal care active ingredients may be covalently attached to the 3′-end of said at least one oligonucleotide. In one aspect, one or more personal care active ingredients may be covalently attached to the 5′-end of said at least one oligonucleotide. In one aspect, one or more personal care active ingredients may be covalently attached to random positions of said at least one oligonucleotide.

Incorporation of modifications to aptamers can be performed using enzymatic or chemical methods. Non-limiting examples of enzymes used for modification of aptamers are terminal deoxynucleotidyl transferases (TdT), T4 RNA ligases, T4 polynucleotide kinases (PNK), DNA polymerases, RNA polymerases, and other enzymes known by those skilled in the art. TdTs are template-independent polymerases that can add modified deoxynucleotides to the 3′ terminus of deoxyribonucleotides. T4 RNA ligases can be used to label ribonucleotides at the 3′-end by using appropriately modified nucleoside 3′,5′-bisphosphates. PNK can be used to phosphorylate the 5′-end of synthetic oligonucleotides, enabling other chemical transformations (see below). DNA and RNA polymerases are commonly used for the random incorporation of modified nucleotides throughout the sequence, provided such nucleotides are compatible with the enzymes.

Non-limiting examples of chemical methods used for modification of aptamers are periodate oxidation of ribonucleotides, EDC activation of 5′-phosphate, random chemical labeling methods, and other chemical methods known by those skilled in the art, incorporated herein.

During periodate oxidation, meta- and ortho-periodates cleave the C—C bonds between vicinal diols of 3′-ribonucleotides, creating two aldehyde moieties that enable the conjugation of labels or active ingredients at the 3′-end of RNA aptamers. The resulting aldehydes can be easily reacted with hydrazine- or primary amine-containing molecules. When amines are used, the produced Schiff bases can be reduced to more stable secondary amines with sodium cyanoborohydride (NaCNBH3).

When EDC activation of 5′-phosphate is used, the 5′-phosphate of oligonucleotides is frequently activated with EDC (1-Ethyl-3-[3-dimethylaminopropyl]carbodiimide hydrochloride) and imidazole to produce a reactive imidazolide intermediate, followed by reaction with a primary amine to generate aptamers modified at the 5′end. Because the 5′ phosphate group is required for the reaction, synthetic oligonucleotides can be first treated with a kinase (e.g. PNK).

Random chemical labeling can be performed with different methods. Because they allow labeling at random sites along the aptamer, a higher degree of modification can be achieved compared to end-labeling methods. However, since the nucleobases are modified, binding of the aptamers to their target can be disrupted. The most common random chemical modification methods involve the use of photoreactive reagents, such as phenylazide-based reagents. When the phenylazide group is exposed to UV light, it forms a labile nitrene that reacts with double bonds and C—H and N—H sites of the aptamers.

Additional information about methods for modification of aptamers is summarized in Hermanson G. T., “Bioconjugate Techniques”, pp. 969-1002, 2nd Edition, Academic Press, San Diego, 2008, the content of which is incorporated herein by reference.

After selection, in addition to chemical modifications, sequence truncations can be performed to remove regions that are not essential for binding or for folding into the structure. Moreover, aptamers can be linked together to provide different features or better affinity. Thus, any truncations or combinations of the aptamers described herein can also be incorporated in the aptamer composition.

### IV. Application of Aptamer Compositions in Personal Health Care Products

Described herein are personal health care compositions and methods for using such compositions for the prevention and treatment of cold-like symptoms due to respiratory tract viral infections. In some aspects, a personal health care composition comprises at least one aptamer as disclosed herein; wherein the at least one aptamer has a binding affinity for ICAM-1 and is configured to reduce the binding of one or more human rhinoviruses to the intercellular adhesion molecule 1 (ICAM-1). The personal health care composition can be preferably applied to areas of the upper respiratory tract, such as the nasal cavity and throat, to provide a barrier to rhinovirus binding and entrance into cells.

The personal health care composition preferably comprises a pharmaceutically effective amount of at least one aptamer. In some aspects, the personal health care composition can comprise between about 0.001% to about 1% of the at least one aptamer, alternatively from about 0.005% to about 0.5%, alternatively from about 0.01% to about 0.1%, all by weight of the composition.

The personal health care compositions can be administered orally or intranasally. In one aspect, the personal health care composition can be an oral composition. An oral composition can be in liquid form, semi-solid form, suspension form, or in any solid form that is capable of quickly dissolving in the mouth. Non-limiting examples of oral dosage forms can include tablets, lyophilized tablets, lollipops, lozenges, liquid center-filled confectioneries, candies, powders, granular substances, films, liquids, solutions, suspensions, mouth rinses or gargles, saline washes, dispersible fluids, sprays, quick dissolving fibers, such as polyvinylpyrrolidone and poly(vinyl alcohol), and combinations thereof. Solid oral dosage forms can be of any desired size, shape, weight, consistency or hardness, bearing in mind that it should not be swallowed before it disintegrates and can easily fit inside the mouth. Alternatively, the personal health care composition can be a nasal composition. A nasal composition can be in any dosage form capable of quickly dispersing in the nose. Non-limiting examples of nasal dosage forms can include vapors, creams, ointments, powders, granular substances, films, liquids, dispersible fluids, sprays, and combinations thereof.

As used herein, the term “administering” with respect to a human/mammal means that the human/mammal ingests or is directed to ingest, or does ingest, or deliver, or chew, or drink, or spray, or place in mouth or nose, or inhale one or more of the personal health care compositions. Administration may be on an as-needed or as-desired basis, for example, once-weekly, or daily, including multiple times daily, for example, at least once daily, at least twice daily, at least three times daily, or at least four times daily.

The personal health care compositions may be administered to prevent and treat cold-like symptoms. As used herein “cold-like symptoms” refer to symptoms typically associated with respiratory tract viral infections. These symptoms include, but are not limited to, nasal congestion, chest congestion, sneezing, rhinorrhea, fatigue or malaise, coughing, fever, sore throat, headache, and other known cold symptoms.

As further used herein, “treat” or “treatment” with respect to respiratory illness means that administration of the referenced composition prevents, alleviates, ameliorates, inhibits, or mitigates one or more symptoms of the respiratory illness or the respiratory illness itself, or any like benefit with respect to the respiratory illness in a mammalian subject in need thereof, preferably in humans. As such, this includes, for example: preventing a respiratory illness or its associated symptoms from occurring in a mammal, for example when the mammal is predisposed to acquiring the respiratory illness, but has not yet been diagnosed with the illness; inhibiting the respiratory illness or its associated symptoms; and/or alleviating, reversing, or curing the respiratory illness or its associated symptoms. Insofar as the methods of the present invention are directed to preventing a respiratory illness, it is understood that the term “prevent” does not require that the respiratory illness be completely thwarted. Rather, as used herein, the term “preventing” or the like refers to the ability of the skilled artisan to identify susceptibility to respiratory illness (such as, for example, in humans during winter months), such that administration of the referenced compositions may occur prior to the onset of the symptoms associated with the illness.

The personal health care compositions and methods of the present invention can comprise, consist of, or consist essentially of, the essential elements and limitations of the invention described herein, as well as any additional or optional ingredients, components, or limitations described herein or otherwise useful in personal health care compositions intended for use by a subject.

All parts, percentages, and ratios herein are by weight unless otherwise specified. All such weights as they pertain to listed ingredients are based on the active level and, therefore do not include solvents or by-products that may be included in commercially available materials, unless otherwise specified. All measurements referred to herein are made at 25° C. unless otherwise specified.

The personal health care compositions of the present invention may include one or more of the following:

The personal health care composition can comprise a solvent. Non-limiting examples of solvents include water, propylene glycol, ethanol, glycerin, polyethylene glycol, and combinations thereof. Solvent can be present in an amount of from about 2% to about 99%, by weight of the composition, alternatively from about 5% to about 95%, alternatively from about 10% to about 80, alternatively from about 12% to about 65%, alternatively from about 20% to about 50%.

The personal health care composition can comprise a thickening agent. Non-limiting examples of thickening agents can include carboxymethylcellulose (CMC), carboxymethylcellulose sodium; and mixtures thereof. When present, the composition can comprise from about 0.01% to about 60% of a thickening agent, alternatively from about 0.1% to about 40%, alternatively from about 1% to about 30%, alternatively from about 2% to about 20%, alternatively from about 3% to about 15%, all by weight of the composition. In one aspect, the thickening agent can provide a moisturizing and/or hydration benefit that relieves the cough on contact and/or provides aid in healing the mouth and/or throat.

The personal health care composition can comprise a diluent. Non-limiting examples of diluents can include microcrystalline cellulose, silicified microcrystalline cellulose, such as ProSolv® SMCC 90 (commercially available from JRS Pharma, Patterson, NY, USA), dextrose, mannitol, sorbitol, maltodextrin, maltitol, and combinations thereof. Suitable diluent levels are from about 20% to about 90% diluent, by weight of the composition, alternatively from about 30% to about 85%, alternatively from about 40% to about 83%, alternatively from about 50% to about 80%, alternatively from about 60% to about 78%.

The personal health care composition can comprise a disintegrant. A disintegrant can be included to formulate a rapid disintegration of the solid oral dosage form following administration. Non-limiting examples of disintegrants can include crospovidone, sodium starch glycolate, crosslinked sodium carboxymethyl cellulose, low substituted hydroxypropylcellulose, guar gum, sodium alginate, and mixtures thereof. Suitable disintegrant levels are from about 1% to about 20%, by weight of the composition, alternatively from about 2% to about 15%, alternatively from about 3% to about 10%, alternatively from about 5% to about 8%.

In one aspect, a composition can comprise mannitol and crospovidone to provide quick disintegration and dissolution. One advantage to using a soluble sugar, like mannitol, is that it can pick up water and dissolve quickly. One advantage to using a disintegrant, like crospovidone, is that it can absorb water and swell, thus causing the dosage form to break apart. As a dosage form breaks apart it is exposed to liquid, such as saliva in the oral cavity, and can dissolve faster. The ratio of mannitol to crospovidone can be about 15:1, alternatively about 13:1 alternatively about 10:1.

The personal health care composition can comprise a lubricant. Non-limiting examples of lubricants can include sodium stearyl fumarate, magnesium stearate, calcium stearate, zinc stearate, stearic acid, glyceryl behenate, hydrogenated vegetable oils, talc, polyethylene glycol, mineral oil, and combinations thereof. Suitable levels of lubricant are from about 0.05% to about 5% lubricant, by weight of the composition, alternatively from about 0.1% to about 3%, alternatively from about 0.25% to about 1.5%, alternatively from about 0.3% to about 1%, alternatively from about 0.4% to about 0.6%.

In one aspect, the personal health care composition can be a non-Newtonian, or thixotropic, fluid, exhibiting a reduced apparent viscosity while being subjected to shear forces, but a high apparent viscosity while at rest. One advantage to a non-Newtonian fluid is that it permits application by spraying with a pump spray device or squeeze-type spray bottle immediately following the application of a shearing force (such as those created by vigorously shaking the device) but causes the sprayed material to remain at least temporarily relatively immobile on mucosal membranes or the skin. Preferably, the composition can have a very rapid rate of viscosity recovery following withdrawal of the shearing force.

The personal health care composition can comprise a rheology-modifying agent. Non-limiting examples of rheology-modifying agents can include sodium carboxymethyl cellulose, algin, carrageenans (including iota, kappa, lambda carrageenan, and combinations thereof), carbomers, galactomannans, hydroxypropyl methylcellulose, hydroxypropyl cellulose, polyethylene glycols, polyvinyl alcohol, polyvinylpyrrolidone, sodium carboxymethyl chitin, sodium carboxymethyl dextran, sodium carboxymethyl starch, microcrystalline cellulose, mixtures of microcrystalline cellulose and carboxymethylcellulose sodium (commercially available as Avicel® RC-591 from FMC Corporation, Philadelphia, Pa), xanthan gum, and combinations thereof. Suitable levels of rheology-modifying agents can be from about 0.5% to about 15%, alternatively from about 1% to about 12%, alternatively from about 2% to about 6%, all by weight of the composition. Rheology-modifying agents can not only provide viscosity benefits but can also coat the nose and throat longer to sooth and/or deliver an agent of choice.

The personal health care composition may further comprise a humectant. Humectants, which can be hygroscopic materials such as glycerin, a polyethylene or other glycol, a polysaccharide, aloe, and the like, act to inhibit water loss from the composition and may add moisturizing qualities.

The personal health care composition can comprise an acidic agent. The acidic agent can comprise organic acids, pyroglutamic acid, and combinations thereof. Suitable organic acid can include, but are not limited to, ascorbic acid, monocarboxylic acids, dicarboxylic acids, tricarboxylic acids, and mixtures thereof. Specific non-limiting examples of suitable monocarboxylic, dicarboxylic, or tricarboxylic acids include salicylic, fumaric, benzoic, glutaric, lactic, citric, malonic, acetic, glycolic, malic, adipic, succinic, aspartic, phthalic, tartaric, glutamic, gluconic, and mixtures thereof. Without being limited by theory, it is believed that incorporating acids in a nasal composition can create a hostile environment for viruses without significantly irritating specific areas of the respiratory tract such as the nasal tissues. The composition can comprise from about 0.01% to about 10% organic acid, alternatively from about 0.05% to about 5%, alternatively from about 0.10% to about 2.5%, all by weight of the composition.

The personal health care composition can comprise a surfactant spreading aid such as polyoxyethylene (20) sorbitan mono-oleate, commercially sold as Polysorbate 80, Polyoxyethylene (20) sorbitan monolaurate, commercially sold as Polysorbate 20, Polyoxyl 400 stearate, polyethylene glycol, Polyethylene-polypropylene glycol, commercially sold as Poloxamer 407, and combinations thereof. The surfactants can be included in the composition at concentrations ranging from about 0.001% to about 10%, alternatively from about 0.01% to about 5%, alternatively from about 0.1% to about 3%, by weight of the composition.

### Additional Components

The personal health care composition described herein may optionally comprise one or more additional components known for use in personal health care products, provided that the additional components are physically and chemically compatible with the components described herein, or do not otherwise unduly impair product stability, aesthetics, or performance. Optional components suitable for use herein include materials such as preservatives, pH adjusting agents, chelating agents, metal compounds, pharmaceutical active ingredients, vitamins, herbal ingredients, sweeteners, sensates, flavoring agents, natural honey, volatile oils, aromatic components such as camphor, eucalyptol, menthol, fragrances and the like, antioxidants, amino acids, energy boosting ingredients, sleep aids, sodium chloride, and combinations thereof. The optional components can be included in the personal health care composition at concentrations ranging from about 0.001% to about 20%, alternatively from about 0.01% to about 10%, alternatively from about 0.1% to about 5%, all by weight of the composition.

In one aspect, the personal health care composition can comprise a preservative. Preservatives can optionally be included to prevent microbial contamination. Non-limiting examples of preservatives can include benzalkonium chloride, chlorhexidine gluconate, phenyl ethyl alcohol, phenoxyethanol, benzyl alcohol, sorbic acid, thimerosal, phenylmercuric acetate, methylparaben, propylparaben, butylparaben, chlorobutanol, and mixtures thereof.

In one aspect, the personal health care composition can comprise a pH adjusting agent. Non-limiting examples of pH adjusting agents can include sodium bicarbonate, sodium phosphate, sodium hydroxide, ammonium hydroxide, sodium stannate, triethanolamine, sodium citrate, disodium succinate, and mixtures thereof. Optional pH adjusting agents can be included in the composition to adjust the pH to a value of from about 2 to about 8, alternatively from about 2 to about 5. If present, the pH adjusting agents are generally included at concentrations ranging from about 0.01 to about 5.0%, by weight of the composition.

In one aspect, the personal health care composition can comprise a chelating agent. Non-limiting examples of suitable optional chelating agents can include phytic acid, disodium and calcium salts of ethylene diamine tetraacetic acid (EDTA), tetrasodium EDTA, sodium hexametaphosphate (SHMP), di(hydroxyethyl)glycine, 8-hydroxyquinoline, and mixtures thereof. The chelating agents can be included at concentrations ranging from about 0.001% to 10%, preferably from about 0.005% to about 5%, more preferably from about 0.01% to about 2%, by weight of the composition.

The personal health care composition can comprise a metal compound. Metal compounds suitable for use herein include those metal compounds containing a metal ion selected from the group consisting of manganese (Mn), silver (Ag), zinc (Zn), tin (Sn), iron (Fe), copper (Cu), aluminum (Al), nickel (Ni), cobalt (Co), and mixtures thereof. Non-limiting examples of a metal compound suitable for use herein include zinc acetate, zinc chloride, zinc ascorbate, zinc gluconate, zinc pidolate, zinc succinate, zinc sulphate, zinc edetate, and mixtures thereof. Zinc acetate is the most preferred metal compound.

When the personal health care composition comprises a metal compound containing a zinc ion, it is believed that the zinc ion provides for antiviral properties. Zinc ions have been shown to be both antiviral and antibacterial. They are believed to inhibit cleavage of rhinovirus polypeptides, preventing replication and formation of infective virions. Zinc ions reduce the ability of rhinoviruses to penetrate cell membranes, partly by lowering expression of intercellular adhesion molecule ICAM. Zinc ions have also been shown to stimulate T-cell lyphocytes, including production of the natural antiviral, interferon-gamma. They stabilize cell plasma membranes, protecting cells from cytotoxic agents, and preventing cell leakage. Furthermore, it is known that metal ions such as iron, silver, copper, and zinc can provide antiviral properties for the prevention and treatment of cold and influenza-like symptoms. The concentration of the metal compound in the personal health care compositions can range from about 0.001% to about 20%, alternatively from about 0.01% to about 10%, alternatively from about 0.05% to about 5%, alternatively from about 0.1% to about 2%, alternatively from 0.2% to about 1%, all by weight of the composition.

Non-limiting examples of pharmaceutical active ingredients can include menthol; anesthetics such as benzocaine and lidocaine; decongestants such as phenylephrine, pseudoephedrine, xylometazoline, and oxymetazoline; antihistamines such as doxylamine, diphenhydramine, loratadine, and cetirizine; expectorants such as guaifenesin, ambroxol, and bromhexine; pain relievers such as acetaminophen (APAP), ibuprofen, ketoprofen, diclofenac, naproxen, and aspirin; antitussives such as dextromethorphan, codeine, chlophedianol, and levodropropizine; the free and addition salts thereof; and combinations thereof. Pharmaceutical active ingredients can be present at a level from about 0.01% to about 25%, alternatively from about 0.05% to about 15%, alternatively from about 0.1% to about 10%, from about 1% to about 5%, all by weight of the composition. In one aspect, the personal healthcare composition can comprise at least one aptamer and one or more pharmaceutical active ingredients to provide relief of one or more symptoms and inhibit rhinovirus binding.

Non-limiting examples of vitamins can include Vitamin A, Vitamin C, Vitamin D2, Vitamin D3, Vitamin E, Vitamin K1, Vitamin K3, Vitamin B1, vitamin B3, folic acid, Vitamin B12, Vitamin B3, Vitamin B7, and combinations thereof. In some aspects, the composition can comprise from about 0.1 to about 10% vitamins, alternatively from about 1 to about 8%, alternatively from about 2 to about 6%, all by weight of the composition.

Non-limiting examples of herbal ingredients can include rosemary (leaf), ginger, lemon balm, green tea, holy basil, oregano, thyme, ashwagandha, bacopa, chamomile, valerian, rosemary, turmeric, grapeseed, blueberry, coffee, curcumin, elderberry, marshmallow root, ivy leaf, black tea, white tea, oolong tea, green tea, and combinations thereof. In some aspects, the herbal ingredient can be whole herbs or plant parts, extracts, powders, concentrates, or combinations thereof. In some aspects, the composition can comprise from about 0.1 to about 10% herbal ingredients, alternatively from about 1 to about 8%, alternatively from about 2 to about 6%, all by weight of the composition.

In one aspect, the sweetener can be selected from the group comprising sugar alcohols, synthetic sweeteners, high intensity natural sweeteners, and combinations thereof.

Non-limiting examples of nutritive sweeteners can include sucrose, dextrose, glucose, fructose, lactose, tagatose, maltose, trehalose, high fructose corn syrup, and combinations thereof. Nutritive sweeteners can be present in an amount from about 1% to about 99%, by weight of the composition, alternatively from about 4% to about 95%, alternatively from about 10% to about 70%, alternatively from about 15% to about 60%, alternatively from about 25% to about 50%, in another example about 35% to about 45%.

Non-limiting examples of sugar alcohols can include xylitol, sorbitol, mannitol, maltitol, lactitol, isomalt, erythritol, and combinations thereof. Sugar alcohols can be present in an amount from about 5% to about 70%, by weight of the composition, alternatively from about 10% to about 60%, alternatively from about 15% to about 55%, alternatively from about 25% to about 50%, alternatively from about 30% to about 45%.

Non-limiting examples of synthetic sweeteners can include aspartame, acesulfame potassium, alitame, sodium saccharin, sucralose, neotame, cyclamate, and combinations thereof. Synthetic sweeteners can be present in an amount from about 0.01% to about 10%, by weight of the composition, alternatively from about 0.05% to about 5%, alternatively about 0.1% to about 3%, alternatively from about 0.2% to about 1%, alternatively from about 0.1% to about 0.5%.

Non-limiting examples of high intensity natural sweeteners can include neohesperidin dihydrochalcone, stevioside, rebaudioside A, rebaudioside C, dulcoside, monoammonium glycyrrhizinate, thaumatin, and combinations thereof. High intensity natural sweeteners can be present in an amount from about 0.01% to about 10% by weight of the composition, alternatively about 0.05% to about 5%, alternatively from about 0.1% to about 3%, alternatively from about 0.5% to about 1%.

The personal health care composition can comprise a flavoring system comprising sensates, flavoring agents, salivating agents, and combinations thereof.

The personal health care composition can comprise a sensate. Non-limiting examples of sensates can include cooling sensates, warming sensates, tingling sensates, and combinations thereof. Sensates can deliver sensory signals to the mouth, throat, nasal, and/or sinus passages so that the personal health care composition may be perceived by the user as immediately acting to alleviate an ailment and/or to provide a soothing sensation.

Non-limiting examples of cooling sensates can include WS-23 (2-Isopropyl-N,2,3-trimethylbutyramide), WS-3 (N-ethyl-p-menthane-3-carboxamide), WS-30 (1-glyceryl-p-menthane-3-carboxylate), WS-4 (ethyleneglycol-p-methane-3-carboxylate), WS-14 (N-t-butyl-p-menthane-3-carboxamide), WS-12 (N-(4-,ethoxyphenyl)-p-menthane-3-carboxamide), WS-5 (ethyl 3-(p-menthane-3-carboxamido)acetate), menthol, levomenthol, 1-menthone glycerol ketal (sold as Frescolat® MGA by Symrise, Holzminden, Germany), (−)-Menthyl lactate (sold as Frescolat® ML by Symrise, Holzminden, Germany), (−)-Menthoxypropane-1,2-diol (sold as Coolact® 10 by Vantage Specialty Ingredients, Inc., Warren, NJ), 3-(1-menthoxy)-2-methylpropane-1,2-diol, (−)-Isopulegol (sold as Coolact P® by Takasago International, Tokyo, Japan), cis & trans p-Menthane-3,8-diols (sold Coolact® 38D by Takasago International), menthyl pyrrolidone carboxylate (sold as Questice® by Givaudan Active Beauty, Verbuer, Switzerland), (lR,3R,4S)-3-menthyl-3,6-dioxaheptanoate (available from Firmenich, Geneva, Switzerland), (lR,2S,5R)-3-menthyl methoxyacetate (available from Firmenich), (lR,2S,5R)-3-menthyl 3,6,9-trioxadecanoate (available from Firmenich), (lR,2S,5R)-menthyl 11-hydroxy-3,6,9-trioxaundecanoate (available from Firmenich), (lR,2S,5R)-3-menthyl (2-hydroxyethoxy)acetate (available from Firmenich), Icilin also known as AG-3-5 (chemical name 1-(2-hydroxyphenyl)-4-(3-nitrophenyl)-3,6-dihydropyrimidin-2-one), 4-methyl-3-(1-pyrrolidinyl)-2[5H]-furanone, Peppermint oil, Spearmint oil, L-Monomenthyl succinate, L-monomenthyl glutarate, 2-1-menthoxyethanol (Coolact® 5), 3-1-Menthoxy propane-1,2-diol (sold as TK10 by Takasago International), N-(4-cyanomethylphenyl)-p-menthanecarboxamide (sold as Evercool™ 180 by Givaudan), and combinations thereof. Cooling sensates can be present from about 0.001% to about 1%, by weight of the composition, alternatively from about 0.01% to about 0.5%, alternatively from about 0.02% to about 0.25%, alternatively from about 0.03% to about 0.10%.

Non-limiting examples of warming sensates can include vanillyl alcohol n-butyl ether (sold as TK-1000 by Takasago International), Heatenol™ (available from Sensient Pharmaceutical, St. Louis, MO), Optaheat (sold by Symrise, Holzminden, Germany), ginger extract, capsicum tincture, cinnamon, capsaicin, curry, Isobutavan, Nonivamide, vanillyl butyl ether (commercially available as Hotact® VBE), piperine, and combinations thereof. Warming sensates can be present from about 0.005% to about 2%, by weight of the composition, alternatively from about 0.01% to about 1%, and alternatively from about 0.1% to about 0.5%.

Non-limiting examples of flavoring agents can include natural flavoring agents, artificial flavoring agents, artificial extracts, natural extracts and combination thereof. Non-limiting examples of flavoring agents can include vanilla, honey, lemon, lemon honey, cherry vanilla, peach, honey ginger, chamomile, cherry, cherry cream, mint, vanilla mint, dark berry, black berry, raspberry, peppermint, spearmint, honey peach, acai berry, cranberry, honey cranberry, tropical fruit, dragon fruit, wolf berry, red stem mint, pomegranate, black current, strawberry, lemon, lime, peach ginger, orange, orange cream, apricot, anethole, ginger, jack fruit, star fruit, blueberry, fruit punch, lemon grass, banana, strawberry banana, grape, blue raspberry, lemon lime, wintergreen mint, bubble gum, tart honey lemon, green apple, apple, tangerine, grapefruit, kiwi, pear, tangerine, tangerine lime, menthol, and combinations thereof. Flavoring agents can be present from about 0.05% to about 10%, by weight of the composition, alternatively from about 0.1% to about 8%, alternatively from about 0.2% to about 6%, alternatively from about 0.4% to about 3%, alternatively from about 0.6% to about 1.5%.

Also described herein is a kit comprising the personal health care composition described herein. In one aspect, the kit can comprise a delivery device and the personal health care composition contained in the delivery device. In one aspect, the kit can optionally comprise at least one additional component, such as a supplement or a vitamin composition.

Also described herein is a method of providing one or more health benefits comprising administering a personal health care composition as described herein comprising an aptamer to a subject in need thereof, wherein the aptamer has a binding affinity for ICAM-1. Non-limiting examples of the one or more health benefits can include providing a physical barrier to block rhinovirus binding and entering cells, helping to stop a cold caused by rhinovirus from forming, reducing the severity and/or duration of a cold caused by rhinovirus, reducing the chances of getting a cold, and combinations thereof.

### EXAMPLES

The following examples illustrate non-limiting examples of the invention described herein. The exemplified personal health care compositions can be prepared by conventional formulation and mixing techniques. It will be appreciated that other modifications of the personal health care compositions within the skill of those in the formulation art can be undertaken without departing from the spirit and scope of this invention.

The following are non-limiting examples of personal health care compositions described herein.

### ORAL COMPOSITION EXAMPLES

### Throat Spray

| | Ex. 1 | Ex. 2 |
|---|---|---|
| | (Wt %) | (Wt %) |
| Benzocaine | 5.0 | 0 |
| Menthol | 1.0 | 1.0 |
| Glycerin | 17.0 | 17.0 |
| Flavoring system | 0.15 | 0.15 |
| Propylene Glycol | 65.0 | 65.0 |
| Ethyl Alcohol 95% | 7.99 | 7.99 |
| Saccharin Sodium | 0.13 | 0.13 |
| Sucralose | 0.18 | 0.18 |
| Color | 0.005 | 0.005 |
| Aptamer | 0.001-1.0 | 0.001-1.0 |
| Water | Q.S. | Q.S. |

### Orally Dissolving Tablet Formula

| | Ex. 3 | Ex. 4 | Ex. 5 | Ex. 6 | Ex. 7 |
|---|---|---|---|---|---|
| | (Wt %) | (Wt %) | (Wt %) | (Wt %) | (Wt %) |
| Mannitol | 59.5 | 49.5 | 39.5 | 39.5 | 39.5 |
| Sucrose | 4.0 | 4.0 | 4.0 | 4.0 | 4.0 |
| Crospovidone | 4.0 | 4.0 | 4.0 | 4.0 | 4.0 |
| ProSolv ® | Q.S. | Q.S. | Q.S. | Q.S. | Q.S. |
| SMCC 90 | | | | | |
| Diphenhydramine | 0 | 12.5 | 12.5 | 12.5 | 12.5 |
| HCl (Active) | | | | | |
| Sodium Caprate | 0 | 0 | 0 | 1.0 | 0 |
| Cetylpyridinium | 0 | 0 | 0 | 0 | 1.0 |
| Chloride | | | | | |
| Magnesium | 1.0 | 1.0 | 1.0 | 1.0 | 1.0 |
| Stearate | | | | | |
| Aptamer | 0.001-1.0 | 0.001-1.0 | 0.001-1.0 | 0.001-1.0 | 0.001-1.0 |

### Liquid Composition

| | Ex. 8 | Ex. 9 |
|---|---|---|
| | (Wt %) | (Wt %) |
| Phenylephrine HCl | 0.031 | 0 |
| Acetaminophen | 2.01 | 0 |
| Dextromethorphan | 0.06 | 0 |
| Guaifenesin | 1.24 | 0 |
| Propylene glycol | 23.02 | 23.02 |
| Glycerin Solution (96%) | 8.00 | 8.00 |
| Sorbitol Solution (70%) | 13.15 | 13.15 |
| Xanthan gum | 0.15 | 0.15 |
| Sodium citrate dihydrate | 0.20 | 0.20 |
| Citric acid USP | 0.22 | 0.22 |
| Sodium benzoate | 0.10 | 0.10 |
| Saccharin sodium | 0.20 | 0.20 |
| Sucralose | 0.20 | 0.20 |
| Flavor | 0.001-0.6 | 0.001-0.6 |
| Color | 0.02 | 0.02 |
| Water | Q.S. | Q.S. |
| Aptamer | 0.001-1.0 | 0.001-1.0 |

### Throat Lozenge Composition

| | Ex. 10 |
|---|---|
| | (Wt %) |
| Menthol | 0.2882 |
| Color | 0.1 |
| Ascorbic Acid | 0.26 |
| Sucrose | Q.S. |
| Liquid Glucose | 33.26 |
| Flavor | 0-0.6 |
| Aptamer | 0.001-1.0 |

### Nasal Compositions

### Saline Nasal Spray Composition

| | Ex. 11 |
|---|---|
| | (Wt %) |
| Water | Q.S. |
| Sodium Chloride | 2.0 |
| Aloe | 0-1.0 |
| Sodium Bicarbonate | 0-2.0 |
| Eucalyptus Oil | 0-0.3 |
| Aptamer | 0.001-1.0 |

### Nasal Spray Compositions

| | Ex. 12 | Ex. 13 |
|---|---|---|
| Ingredient | (Wt %) | (Wt %) |
| Water | Q.S. | Q.S. |
| Avicel ™ 591 | 3 | 3 |
| Polyvinylpyrrolidone | 3 | 3 |
| Carbowax ™ PEG 1450 | 5 | 5 |
| Sodium phosphate, dibasic | 0.0975 | 0.0975 |
| Sodium phosphate, monobasic | 0.5525 | 0.5525 |
| Levomenthol | 0.027 | 0.027 |
| Eucalyptol | 0.009 | 0.009 |
| Camphor | 0.009 | 0.009 |
| Benzalkonium Chloride | 0.1471 | 0.1471 |
| 50% Solution | | |
| Benzyl Alcohol | 0.35 | 0.35 |
| Disodium EDTA | 0.03 | 0.03 |
| Oxymetazoline HCl | 0.05 | 0 |
| Aptamer | 0.001-1.0 | 0.001-1.0 |

### Additional Nasal Spray Compositions

| | Ex. 14 | Ex. 15 | Ex. 16 |
|---|---|---|---|
| | (Wt %) | (Wt %) | (Wt %) |
| Pyroglutamic Acid | 0.35 | 0.70 | 1.00 |
| Succinic Acid | 1.00 | 0.70 | 0.35 |
| Zinc Acetate Dihydrate | 0.12 | 0.012 | 0.12 |
| Polysorbate 80 | 0.05 | 0.05 | 0.05 |
| Carbopol 980 | — | — | 1.20 |
| Hydroxypropyl methyl | 1.20 | — | — |
| cellulose | | | |
| Poloxamer 407 | — | 15.8 | — |
| Sodium Saccharin | — | 0.025 | 0.025 |
| Sucralose | 0.025 | — | — |
| Phenyl ethyl alcohol | 0.37 | 0.37 | 0.35 |
| Sodium chloride | 0.20 | 0.20 | 0.50 |
| Camphor | — | 0.03 | — |
| Menthol | 0.02 | 0.06 | 0.02 |
| Eucalyptol | — | 0.02 | — |
| Aromatic System | 0.05 | 0.38 | 0.05 |
| Sodium Hydroxide (30%) | — | — | 0.10 |
| Disodium succinate | 1.00 | 0.50 | — |
| Water | Q.S. | Q.S. | Q.S. |
| Aptamer | 0.001-1.0 | 0.001-1.0 | 0.001-1.0 |

### V. Examples

### Example 1. Aptamer Selection and Next Generation Sequence Characterization

### A. Selection Strategy

One objective of this invention was to develop aptamers that would not just specifically bind to ICAM-1 receptors but would do so in a way that would block or inhibit the binding of virus particles to the receptor protein. The selection of aptamers against the extracellular domain of the ICAM-1 receptor alone would not necessarily be sufficient to block virus binding to the same protein as aptamers are relatively small and their blocking footprint will be limited to the epitopes that they bind to. If the epitopes that the aptamer binds to are not involved in virus binding to the ICAM-1 receptor, they will not inhibit binding of the virus particles.

This objective was consciously incorporated into the selection strategy, first by including several rounds of positive selection against the exo-cellular domain of the ICAM-1 protein (SEQ ID NO: 214); secondly, by imposing a double positive selection such that aptamers would be enriched for binding to the ICAM-1 extra-cellular domain in the context of nasal cells; thirdly, by imposing counter selection against HEK293 cells that carry similar receptor proteins (ICAM-3 and ICAM-5); and fourthly, by performing selection channels against specific desirable and undesirable aptamer binding outcomes including, specific elution of bound aptamers from nasal cells with the addition of rhinovirus particles, blocking of aptamer binding to ICAM-1 cells by the pre-application of rhinovirus particles, positive selection against HEK293 cells, positive selection against the extra-cellular domain of ICAM-1, and double positive selection against the extra-cellular domain of ICAM-1 and nasal cells.

Double positive selection (extra-cellular domain of ICAM-1 and nasal cells) ensures that enriched aptamers are favored that bind to the ICAM-1 receptor as it is presented on nasal cells. If selection was only performed against the extra-cellular domain of ICAM-1, it is possible that epitopes would be present that are not present in vivo. If selection was only performed against nasal cells, it is possible that aptamers would be enriched for binding targets other than ICAM-1 on the surface of such cells.

The counter selection against HEK293 cells was implemented to drive enrichment of aptamers that bound to the N-terminus of the ICAM-1 extracellular domain. HEK293 cells express other members of the ICAM receptor family, ICAM-3 and ICAM-5. These receptor proteins differ in their extracellular domain from ICAM-1 predominantly at their N-terminus. The N-terminus of the ICAM-1 receptor is the region of the extra-cellular domain that rhinovirus particles bind to. Thus, this counter selection step was included to drive aptamer selection towards those aptamers that will block or inhibit rhinovirus binding to nasal cells.

Finally, once the aptamer library was enriched with double positive selection against the extra-cellular domain of ICAM-1 and nasal cells, and counter selection against HEK293 cells, the enriched library was separated into aliquots and applied to several different targets, including continued double positive selection, positive selection against HEK293 cells, positive selection against the extra-cellular domain alone, selection based on rhinovirus particle elution of aptamers bound to nasal cells, and selection based on blocking aptamer binding to nasal cells through pre-treatment with rhinovirus particles.

Each of these selected libraries was characterized by next generation sequencing. Aptamers that exhibit higher levels of enrichment against the double positive selection, the extracellular domain selection, and either of the rhinovirus particle enabled selection processes and lower enrichment against HEK293 alone would be desirable sequences for the blocking or inhibition of rhinovirus binding to nasal cells.

### B. Growth of Human Cells

### B.1. Human Nasal Epithelial Cells Growth Conditions

Primary human nasal epithelial cells (HNepC; PromoCell, Catalog #C-21060) were grown in airway epithelial cell growth medium (PromoCell, Catalog #C-21160) at 37° C. and 5% CO₂.

### B.2. Growth of HEK293 Cells

HEK293 cells purchased from ATCC (CRL-1573) were grown in Eagle's Minimum Essential Medium (EMEM)+10% Fetal Bovine Serum (FBS) at 37° C. and 5% CO₂.

### B.3. Human Rhinovirus A16 Suspension

UV inactivated HRV16 virus particles were purchased (Zeptometrix Corporation) and stored at −80° C. until use. The concentration of the virus particles (VPs) was calculated to be 98,700 vp/mL.

### C. Aptamer Selection

### C.1. Library Preparation

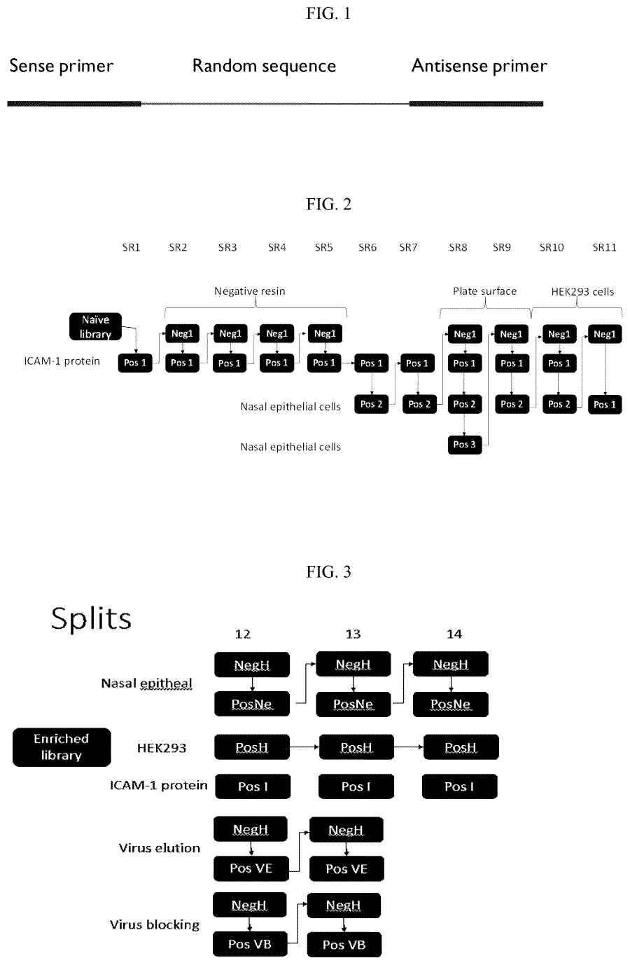

In the first step, a DNA library of about 10¹⁵ different sequences (TriLink BioTechnologies), containing a random region of 40 nucleotides flanked by two conserved regions, forward primer recognition sequence (5′-GGGTGCATCGTTTACGC-3′; SEQ ID No 224) and a 3′ reverse primer recognition sequence (5′-CTGCTGCTGAGGAAGGATATGAG-3′ SEQ ID No 225) (see FIG. 1), was transcribed to RNA using a mixture of 2′-fluoro pyrimidines nucleotides (2F-UTP and 2F-CTP) and natural purine nucleotides.

In brief, about 1.66 nmoles of single stranded DNA were amplified in 390×50 μL PCR reactions for 4 cycles using the primers Lib7_T7 Fwd primer (sequence: 5′-TAATACGACTCACTATAGGGTGCATCGTTTACGC-3′, (SEQ ID No 226) with transcription starting at the first G underlined) and Lib7_Rvs primer (sequence 5′-CTCATATCCTTCCTCAGCAGCAG-3′ SEQ ID No 227). The amplified DNA was purified using the Genejet PCR purification kit (Fisher Scientific, Catalog #K0701). This amplification of the ssDNA library created a dsDNA library with a T7 promoter, which was used as a templated to generate a modified RNA library for selection.

Post DNA amplification, 52 μg of purified dsDNA was transcribed in 26×20 μL transcription reactions by using a mutant T7 polymerase (T7 R&DNA polymerase, Lucigen, Catalog #D7P9205K) polymerase and a mixture of rATP, rGTP and the modified nucleotides 2F-UTP and 2F-CTP. The NTPs were mixed together at a ratio of 3:1 modified to non-modified. Each reaction mixture contained 4 μL 5×T7 R&D polymerase, 1 μL NTP 3:1 mix, 2 μL DTT (0.1M), 0.7 μL T7 R&D polymerase, 1.2 L inorganic pyrophosphatase, 0.5 μL Rnase inhibitor, and 10.6 μL DNA template. The reactions were incubated at 37° C. for 16 hours.

The transcribed library was subjected to Dnase treatment by setting up reaction mixtures consisting of 10 μL 10× Dnase buffer, 4 μL Dnase I, 66 μL Rnase free water, and 20 μL transcription reaction. The reaction mixtures were then incubated at 37° C. for 30 min, 1 μL of 0.5 M EDTA was added and mixed, further incubated at 75° C. for 10 minutes and purified using Monarch RNA cleanup kit (New England Biolabs, Catalog #T2040L).

C.2. Immobilization of ICAM-1 onto His-Pur Ni-NTA Resin

Lyophilized ICAM-1 protein (50 μg Ray-Biotech, Catalog #: 228-21751-2) with a His-tag on the C-terminus region was resuspended in 100 μL of sH₂O (final concentration of 0.5 μg/μL or 9.88 μM). The solution was aliquoted and stored at −20° C. until use. The protein sequence was:

(SEQ ID No 228)
QTSVSPSKVILPRGGSVLVTCSTSCDQPKLLGIETPLPKKELL
LPGNNRKVYELSNVQEDSQPMCYSNCPDGQSTAKTFLTVYWTP
ERVELAPLPSWQPVGKNLTLRCQVEGGAPRANLTVVLLRGEKE
LKREPAVGEPAEVTTTVLVRRDHHGANFSCRTELDLRPQGLEL
FENTSAPYQLQTFVLPATPPQLVSPRVLEVDTQGTVVCSLDGL
FPVSEAQVHLALGDQRLNPTVTYGNDSFSAKASVSVTAEDEGT
QRLTCAVILGNQSQETLQTVTIYSFPAPNVILTKPEVSEGTEV
TVKCEAHPRAKVTLNGVPAQPLGPRAQLLLKATPEDNGRSFSC
SATLEVAGQLIHKNQTRELRVLYGPRLDERDCPGNWTWPENSQ
QTPMCQAWGNPLPELKCLKDGTFPLPIGESVTVTRDLEGTYLC
RARSTQGEVTRKVTVNVLSPRYEVDHHHHHH.

An aliquot of His-Pur Ni-NTA (Fisher Scientific, Catalog #PI88221) resin was transferred to a 0.6 mL tube and centrifuged at 700×g for 2 minutes. The supernatant was removed, and the resin was washed 3 times with 500 μL of PBS buffer (pH 7.4). Then, aliquots of ICAM-1 protein in 1×PBS buffer (pH 7.4) were incubated with the His-Pur Ni-NTA resin overnight at 4° C. while mixing. For selection round 1, 300 pmoles of ICAM-1 protein was immobilized onto 50 μL of resin. For subsequent rounds, 50 pmoles of ICAM-1 protein was immobilized onto 25 μL of resin. After protein immobilization, the resin was transferred to a 1 mL cartridge with a frit filter and washed with 2 mL of 1×PBS buffer. Finally, aliquots of 0.5-1 mM imidazole in 1×PBS buffer were added and incubated with the resin for 30 minutes at 4° C. to block unreacted binding sites on the resin. The resin was washed three times with 1 mL aliquots of 1×PBS buffer.

For negative selections with imidazole blocked resin, aliquots of the His-Pur Ni-NTA resin were incubated with an appropriate concentration of imidazole in 1×PBS buffer for 30 minutes to block unreacted binding sites on the resin, followed by washing with 1× selection buffer. The selection buffer used for all the examples in this application was Dulbecco's PBS buffer supplemented with calcium chloride (CaCl2), 0.9 mM), magnesium chloride (MgCl2 0.49 mM), potassium chloride (KCl, 2.67 mM), potassium phosphate monobasic (KH2PO4, 1.47 mM), sodium chloride (NaCl, 137.93 mM), and sodium phosphate dibasic (Na2HPO4, 8.06 mM).

### C.3. Aptamer Selection Overview

The aptamer selection was performed in fourteen selection rounds (“SR”), which are illustrated in FIG. 2. The selection rounds 1 to 5 enrich the sequences in the aptamer library that bind to ICAM-1 immobilized onto the Ni-NTA Resin. In selection rounds 6 to 9, the aptamer library was subjected to the same ICAM-1 immobilized Ni-NTA Resin procedure and the eluted aptamers were further enriched towards sequences that bind to human nasal epithelial cells (HNepC), this is referred to as double positive selection. In selection rounds 10 to 11, counter selection against HEK293 cells and positive selections against HNepC were performed. Selection rounds 12 to 14, illustrated in FIG. 3, break out to different selection conditions and are referred to as splits. Five different splits were performed: split A: nasal epithelial cells, split B: HEK293 cells, split C: ICAM-1 protein, split D: human rhinovirus A16 (HRV16) elution, and split E: HRV16 blocking.

### C.4. Aptamer Selection Process

### C.4.1 Selection Round 1

The aptamer selection round 1 was completed by performing a positive selection against ICAM-1 immobilized Ni-NTA resin. The RNA library (produced as described in section C.1) was heated to 45° C. for 10 minutes and allowed to cool to room temperature for 10 minutes. Then, the prepared aptamer library was added to 300 pmol of the ICAM-1 immobilized on Ni-NTA resin (prepared as described in section C.2) and incubated with rotation at room temperature for 30 minutes. Unbound RNA was washed off the resin with 500 μL of selection buffer (pH 7.4).

The bound RNA was then eluted twice by adding aliquots of 200 μL of 6 M urea to the resin and incubating the suspension at 85° C. for 5 minutes. The recovered RNA library was collected and purified using Monarch RNA cleanup kit.

The collected aptamer library was reverse transcribed following the Protoscript II Reverse Transcriptase manufacturer's protocol. The number of reverse transcription reactions varied depending on the amount of RNA going into that specific round of selection.

Then, the reverse transcribed aptamer library was amplified by polymerase chain reaction (PCR) using a standard PCR protocol and the following amplification steps:
Step 1: 95° C.—5 minutes
Step 2: 95° C.—10 seconds
Step 3: 56° C.—15 seconds
Step 4: 72° C.—30 seconds
Repeat steps 2 to 4 for 4 cycles
Step 5: 95° C.—10 seconds
Step 6: 59° C.—15 seconds
Step 7: 72° C.—30 seconds
Repeat steps 5 to 7 for up to 26 cycles.

The PCR amplified dsDNA aptamer library was then transcribed back into RNA and subjected to Dnase treatment using the protocols described in section C.1.

### C.4.2 Selection Rounds 2 to 5

Selection rounds 2 to 5 incorporate two selection strategies: negative selection against imidazole blocked Ni-NTA resin and positive selection with ICAM-1 immobilized Ni-NTA resin (see FIG. 2). The negative selection was performed to select aptamer sequences that do not bind to the imidazole blocked Ni-NTA resin (prepared as described in Section C.2). First, an aliquot of 50 μL of imidazole blocked resin was transferred to a 1 mL cartridge fitted with a 20 μm frit and washed twice with 1 mL aliquots of selection buffer. Then, the prepared RNA library from the previous selection round was heated to 45° C. for 10 minutes and allowed to cool to room temperature for 10 minutes. The RNA library was added to the cartridge and incubated at room temperature for 30 minutes with the imidazole blocked Ni-NTA resin. Following incubation, the flow through solution was collected. Then, the cartridge was washed using an aliquot of 500 μL of selection buffer and the solution was collected. The flow through solution and column wash collections were pooled together and purified with Monarch RNA cleanup kit following manufacture protocols.

The RNA library that was obtained from the negative selection was then subjected to the positive selection, which selects for sequences that bind to ICAM-1 immobilized Ni-NTA resin (prepared as described in Section C.2). In brief, the RNA library was heated to 45° C. for 10 minutes and allowed to cool to room temperature for 10 minutes. Then, the RNA library was added to 50 pmoles of the ICAM-1 immobilized on Ni-NTA resin (prepared as described in Section C.2) and incubated with rotation at room temperature for 30 minutes. Unbound RNA was washed off the resin with aliquots of 500 μL of selection buffer. The number of washes varied depending on the selection round and the number of positive selections completed and was pre-determined by selection modelling. Then, the bound RNA library was eluted twice by adding aliquots of 200 μL of 6 M urea to the resin and incubating the suspension at 85° C. for 5 minutes. The eluted RNA library was collected and purified with the Monarch RNA cleanup kit, followed by reverse transcription, PCR amplification, transcription, and DNAse treatment as described in sections C.1 and C.4.1.

### C.4.3. Selection Rounds 6 to 9

The RNA aptamer library that was enriched from selection rounds 1 to 5 was further enriched in selection rounds 6 to 9, which utilizes two selection strategies: a positive selection with ICAM-1 immobilized Ni-NTA resin and another positive selection against human nasal epithelial cells (HNepC) that express the ICAM-1 receptor. This group of selection rounds is referred to as “double positive selection”. In selection round 8, two positive selections against HNepC were performed (i.e. “triple positive selection”).

In selection rounds 6 and 7, the RNA library was resuspended in 500 μL of 1× selection buffer. The first positive selection (selecting against ICAM-1 immobilized Ni-NTA resin) started by adding the resuspended RNA to the ICAM-1 immobilized on Ni-NTA resin, followed by incubation at 37° C. for 30 minutes. The unbound RNA was discarded and the resin was washed with aliquots of 500 μL of 1× selection buffer. For the elution step, an aliquot of 200 μL of 6 M urea was added to the resin and incubated at 85° C. for 5 minutes and the elution solution was collected. The elution step was repeated and the eluants were pooled together and cleaned up using a Monarch RNA clean up kit.

The second positive selection started by preparing the HNepC cells by aspirating the medium from the 6-well plate (˜3 mL) where the cells were grown, followed by washing the cells three times with 3 mL of prewarmed 1× selection buffer. A solution of 1 mL of RNA library in 1× selection buffer was immediately applied to the washed cells and incubated for 30 minutes at 37° C. and 50 revolutions per minute (rpm). After the 30 minute incubation, the supernatant containing ˜50% of the cells was collected, the cells were pelleted at 500×g for 2 minutes and washed twice with 200 μL prewarmed 1× selection buffer. The cell pellet was collected, and the bound RNA was eluted from the cells by the addition of 6 M urea, followed by incubation at 85° C. and RNA purification.

The adhered cells (i.e. remaining ˜50% cells) were washed twice with 1 mL of preheated 1× selection buffer. Then, an aliquot of 1 mL of 10 mM EDTA was added and allowed to incubate with the cells at 37° C. for 15 minutes at 50 rpm. The EDTA treated cells were pelleted at 500×g for 2 minutes. Then, an aliquot of 200 μL of 6 M urea was added to the pellet and the suspension was heated to 85° C. for 5 minutes, followed by centrifugation at 13,000 rpm to recover the RNA aptamers in the supernatant. The elution step was repeated one more time, the eluants were combined, and the RNA aptamers were purified. The reverse transcription, PCR amplification, and transcription following the protocol in sections C.1 and C.4.1 was performed on the purified samples.

In selection rounds 8 and 9, the EDTA lifting of the cells was removed from the protocol and the RNA bound to the cells was eluted using 6 M urea while they were still attached to the 6-well plate. Additionally, a negative selection step was included in both rounds to remove any RNA sequences that bind to the plastic of the 6-well culture plate. For the negative selection, the RNA library was resuspended in 1 mL of 1× selection buffer, followed by heating to 37° C. for at least 10 minutes. One well in a 6-well culture plate was pre-washed twice with 1 mL of 1× selection buffer. Then, the heated RNA library was added to the well and incubated at 37° C. and 50 rpm for 30 minutes. The solution in the well was collected and brought up to 1 mL volume with selection buffer. The resulting 1 mL solution of RNA library was incubated with HNepC, grown in a 6-well plate, at 37° C. at 50 rpm for 1 hour. The unbound RNA was removed from the cells and the cells were washed twice with 1 mL of 1× selection buffer (prewarmed to 37° C.). The bound RNA was eluted by adding 1 mL of 6 M urea and incubating the cells at 85° C. for 5 minutes. The elution step was repeated. The eluants were pooled together and the RNA was purified using the Monarch RNA clean up kit. The selected RNA was reverse transcribed, PCR amplified, transcribed and DNAse treated as previously described.

### C.4.4. Selection Rounds 10 and 11

In selection rounds 10 and 11, a negative selection against HEK293 cells was introduced (see FIG. 2). HEK293 cells do not express the ICAM-1 receptor, which allows for the counterselection of sequences that bind elsewhere on the cell surface that is not ICAM-1.

The HEK293 cells were grown in a 6-well culture plate and were used at 80% confluency or greater. The cells were prepared by removing and discarding all media from the well and by washing the cells three times with 3 mL of pre-warmed 1× selection buffer. Then, the prepared RNA library was added to the cells and the library and cell solution were incubated for 1 hour at 37° C. with gentle shaking (50 rpm). After incubation, the supernatant with the unbound RNA library was removed and collected. Then, the cells were washed with 1 mL of pre-warmed 1× selection buffer and the solution was also collected. The collected RNA solutions were combined and purified with a Monarch RNA Cleanup Kit. This purified RNA library was then subjected to a positive selection round against HNepC, following the same protocol as described on selection rounds 8 and 9 (see section C.4.3). Two positive selections were performed in selection round 10, while a single positive selection was completed in selection round 11.

### C.4.5. Selection Rounds 12 to 14: Nasal Epithelial Cell Split

In the nasal epithelial cell split of selection rounds 12 to 14 (see FIG. 3), the RNA library collected from selection round 11 was further subjected to the negative selection against the HEK293 cells followed by the positive selection with the HNepC, using the protocol described in section C.4.4.

### C.4.6. Selection Rounds 12 to 14: HEK293 Cell Split

In the HEK293 cell split of selection rounds 12 to 14 (see FIG. 3), the RNA library collected from selection round 11 was enriched towards sequences that bind to HEK293 cells. The protocol for this selection round followed the procedure of selection rounds 10 to 11 described in section C.4.4, excluding the selection with the HNepC.

### C.4.7. Selection Rounds 12 to 14: ICAM-1 Protein Split

In the ICAM-1 split of selection rounds 12 to 14 (see FIG. 3), the RNA library collected from selection round 11 was enriched towards sequences that bind to ICAM-1 immobilized onto the Ni-NTA Resin. The protocol for this selection round followed the procedure of selection round 1 described in sections C.1 and C.4.1.

### C.4.8. Selection Rounds 12 to 13: Human Rhinovirus A16 (HRV16) Elution Split

The HRV16 elution split only occurred during selection rounds 12 and 13 (see FIG. 3). The RNA library collected in selection round 11 was further enriched by a negative selection against HEK293 cells followed by a positive selection on HNepC using Human Rhinovirus A16 (HRV16) particles to elute the aptamer library. The negative selection on HEK293 cells followed the same protocol of selection rounds 10 and 11 described in section C.4.4 but excluding the selection against the HNepC.

Following the negative selection with the HEK293 cells, the collected RNA was diluted in 1× selection buffer and heated to 37° C. for 15 minutes. The HNepC cells were washed three times with 1 mL of prewarmed selection buffer and the heated RNA library was added to the cells and incubated for 1 hour at 37° C. and 50 rpm. After incubation, the unbound RNA was removed and discarded. The recovered cells were washed ten times with 1 mL of preheated 1× selection buffer. Then, a suspension of 50% (v/v) virus particles (VPs) (see Section B.3) in 1× selection buffer were mixed with the cells and incubated for 1 hour at 37° C. with 50 rpm mixing. The supernatant was collected, and the RNA was purified and reverse transcribed following the protocol described in sections C.1 and C.4.1.

### C.4.9. Selection Rounds 12 and 13: HRV16 Blocking Split

The HRV16 blocking split was performed during selection rounds 12 and 13 (see FIG. 3). The RNA library of selection round 11 was further enriched by a negative selection against HEK293 cells followed by a positive selection on HNepC with HRV16 bound to the ICAM-1 receptor before exposing the cells to the RNA library. The HEK293 negative selection followed the same protocol of selection rounds 10 and 11 described in section C.4.4, excluding the selection with the HNepC.

Following the negative selection on the HEK293 cells, a suspension of 50% (v/v) virus particles (VPs) in 1× selection buffer was prepared. Then, the suspension was heated to 37° C. for 15 minutes and mixed with prewashed HNepC cells, followed by incubation for 1 hour at 37° C. and 50 rpm. After incubation, all unbound VPs were removed and discarded. Then, the RNA library recovered from the negative selection was resuspended in 1× selection buffer, added to the cells, and incubated at 37° C. for 1 hour. The supernatant containing the unbound RNA was collected, purified and reverse transcribed following the protocols described in sections C.1 and C.4.1.

### D. Aptamers Sequencing

After 14 selection rounds, the aptamer libraries were sequenced. In summary, the selection libraries from rounds 10 to 14 were prepared for next generation sequencing (NGS) through a two-step PCR process. In the first step, a different hex code (6 base sequence) and a portion of a universal sequencing primer was added to the 5′ end of each aptamer library. In the second step, complete universal sequencing primers were added to both ends. After the second PCR step, the libraries were purified through acrylamide electrophoresis and balanced for relative quantity. These libraries were then pooled and sent to the Hospital for Sick Children in Toronto for NGS with an Illumina HiSeq 2500 instrument.

The sequencing data was tabulated and analyzed. A total of 16,116,086 sequences were analyzed and each library contained more than 200,000 sequences. The sequences from selection round 14 (nasal epithelial cell split) were sorted by copy number and named in descending order with the highest copy number sequence being named Nas.R-1. These top sequences are listed in Table 3.

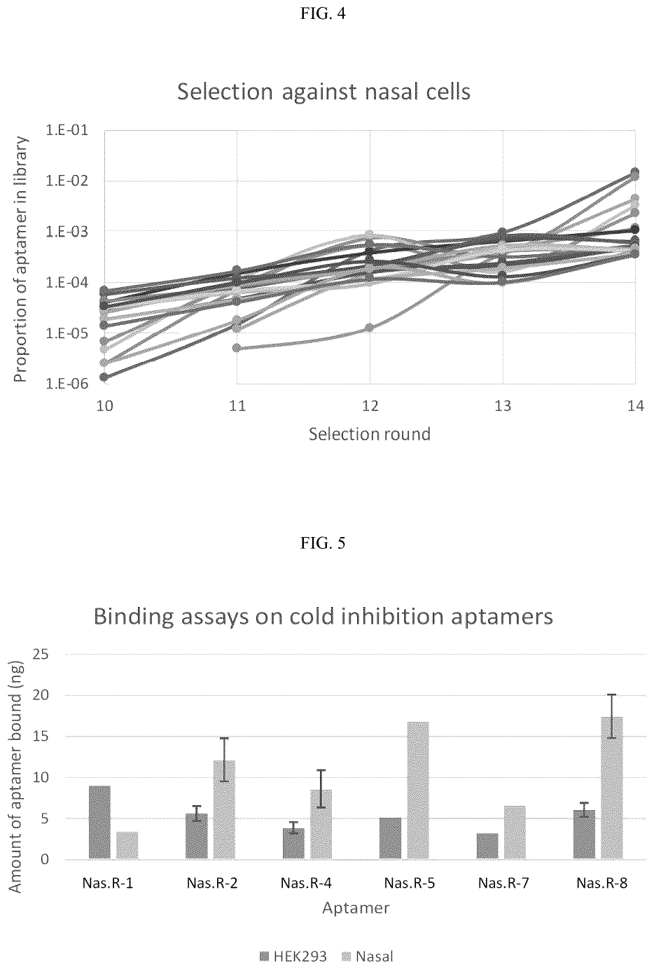

The copy numbers of the top sequences of selection round 14 were determined on the libraries obtained from the other selection rounds. Finally, the frequency was computed for each sequence by dividing observed copy number by the total number of sequences observed in the particular selection library. Enrichment trajectories of the top 20 sequences in terms of frequency across different selection rounds were plotted (see FIG. 4). During the selection, these sequences were enriching at a similar rate.

### Example 2. Aptamer Binding Specificity

It was desired to identify aptamer sequences that bind specifically to the ICAM-1 receptor and block the ability of the rhinovirus from infecting human nasal epithelial cells. The previous section, Example 1, detailed the protocol on the selection process of determining sequences that enriched in the presence ICAM-1. This section will highlight the protocols that were used to determine the sequences discovered in Example 1 that have the highest affinity and specificity towards the ICAM-1 receptor target.

Multiple strategies were implemented to determine the top sequences from selection process for RNA aptamers that bind specifically and with high affinity towards human epithelial cells (HNepC), but not towards HEK293 cells that do not express the ICAM-1 target. The first protocol included exposing HNepC and HEK293 cells to some of the selected aptamer sequences, followed by incubation, elution, and quantification of the concentration of aptamers that bound to each cell type. Another strategy implemented included the visualization and identification of fluorescently labeled RNA aptamers that bind to HNepC, but do not visually bind to HEK293 cells. A final strategy included immobilizing the top RNA aptamer sequences, followed by flowing the exo-cellular domain of the ICAM-1 protein and other various proteins across the aptamer and using plasmon resonance to determine binding affinity. The following section describes in detail the strategies that are summarized above.

A. Detecting Binding Specificity and Affinity via qPCR

### A.1. Synthesis of Aptamer RNA Sequences

DNA oligos that corresponded to the RNA aptamer sense and antisense sequences plus the T7 RNA polymerase promoter were purchased (Integrated DNA Technologies). Each of the oligos were mixed at equimolar concentrations in 10 mM Tris buffer (pH 8.3) containing 50 mM KCl and 1.5 mM MgCl2, followed by incubation at 95° C. for 5 minutes. Then, the modified RNA aptamers were synthesized by transcription of the dsDNA template, followed by DNAse treatment, and purification as described in Example 1 Sections C.1 and C.4.1.

### A.2. RNA Aptamers, HNepC and Hek293 Cell Preparation

The modified RNA aptamers were dissolved at a concentration of 28.2 nM in 1× selection buffer. HNepC or HEK293 cells were grown in a well of a 24-well plate at densities ranging from 70-75% (HNepC) or 90-95% (HEK293 cells) following the protocol outlined in Example 1 Sections B.1 and B.2.

A.3. qPCR Analysis Procedure

For each sample, two 20 μL qPCR reactions were prepared using the Luna qPCR universal mastermix (New England Biolabs, Catalog #M3003L), 0.2 μM of each primer (forward primer: 5′-TAATACGACTCACTATAGGGTGCATCGTTTACGC-3′ (SEQ ID No 226), reverse primer: 5′-CTCATATCCTTCCTCAGCAGCAG 3 (SEQ ID No 227)), and 5 μL of the cDNA sample. qPCR reactions containing known amounts of the sense DNA template were also prepared. The PCR reactions were performed using the following conditions:
Step 1: 95° C. for 3 minutes
Step 2: 95° C. for 15 seconds
Step 3: 56° C. for 15 seconds
Step 4: 60° C. for 30 seconds
Steps 2 to 4 were repeated for 40 cycles.

The Ct values of the binding assay samples were compared to the Ct values of the known amounts of DNA samples to determine the amount of RNA that bound to the cells.

### A.4. Human Nasal Epithelial and HEK293 Aptamer Binding Assay

Six of the top aptamer sequences (Nas.R-1, Nas.R-2, Nas.R-4, Nas.R-5, Nas.R-7 and Nas.R-8) that were identified in the selection process (Example 1) were tested for their binding specificity and affinity towards HNepC or HEK293 cells. The RNA aptamers, HNepC, and HEK293 cells were prepared as described in Section A.2.

The aptamers were incubated with the HNepC for 1 hour at 37° C. and 5% CO₂ with gentle shaking every 15 minutes. The unbound RNA was removed and the cells were washed four times with 150 μL of 1× selection buffer prewarmed at 37° C. To elute the bound RNA aptamers, aliquots of 200 L of 6 M urea were added to the cells, followed by incubation at 85° C. for 5 minutes. The elution step was repeated, the eluants were combined, and the RNA aptamers were purified using a Monarch RNA clean up kit following the manufacture's protocol. Each RNA sample was reverse transcribed in a 20 μL M-MμLV (New England Biolabs, M0253L) reverse transcriptase reaction following the manufacturer's protocol. The reverse transcribed sequences were quantified using qPCR analysis following the protocol described in section A.3. The same procedure was followed for the HEK293 cells. The results are illustrated in FIG. 5. For aptamers Nas.R-2, Nas.R-4, Nas.R-5, Nas.R-7, and Nas.R-8, the binding affinity towards HNepC was higher than for HEK293 cells.

### B.1. Visualizing Aptamer Bound to ICAM-1 on HNepC and HEK293 by Fluorescence

### B.1.1. Preparation of Fluorescently Tagged RNA Aptamers

Modified RNA aptamer Nas.R-4 with a spacer (AAACAAACAAAC; SEQ ID No 235) and a sense binding sequence (GUAUGGCGGUCUCCAACAGG: SEQ ID No 236) at the 3′ end was synthesized, as previously described in section A.1.

(SEQ ID No 229)
5′-GGGUGCAUCGUUUACGCGCAACAUAAA
AAUUUAAAGUGCUCAGUUGUCAAUCUAUG
ACUGCUGCUGAGGAAGGAUAUGAG AAAC
AAACAAAC GUAUGGCGGUCUCCAACAGG-3′

The sense binding sequence was added to anneal to a 6-FAM labelled fluorescent antisense oligonucleotide. Before each binding assay, the NAS-FAM antisense oligo (5′ 6-FAM/CCTGTT GGAGACCGCCATAC-3′ (SEQ ID No 230)) was mixed with the modified RNA aptamer at equimolar concentrations in 1× selection buffer, followed by incubation at 37° C. for 15 minutes.

### B.1.2. HNepC and Hek293 Cell Preparation

HNepC and HEK293 cells were prepared following the procedure outlined in Section A.2 but were seeded at densities of about 50% one to two days before the assay, onto 12 mm glass coverslips (Fisher Scientific, Catalog #12-545-82) submersed in medium in wells of 24-well plates.

### B.1.3. Binding of the Fluorescently Labelled Aptamers to Cells

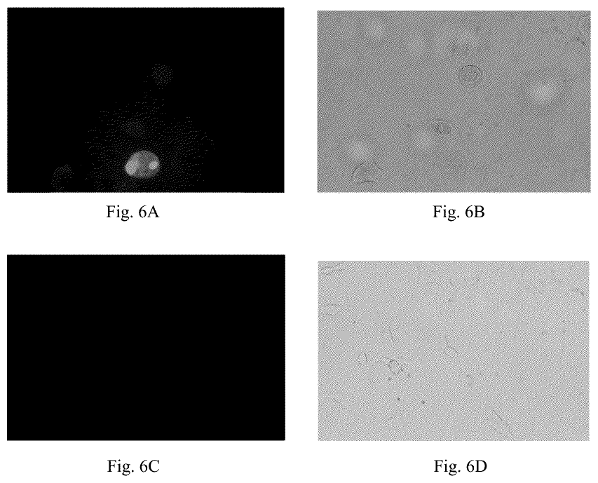

The medium was aspirated from the HNepC culture. Then, an aliquot of 150 μL of the aptamer/NAS-FAM antisense mixture, prepared as described in Section B.1, was applied to the cells, followed by incubation for 15 minutes at 37° C. and 5% CO₂ and with gentle agitation every 5 minutes. The unbound RNA aptamer was aspirated and the HNepC were washed three times with 150 μL of 1× selection buffer prewarmed at 37° C. The coverslip was removed and submersed into a drop of selection buffer on a glass microscope slide. Fluorescence of the cells was monitored for up to about 1 hour using a Nikon inverted fluorescent microscope and a FITC fluorescence filter. Images (see FIGS. 6A-6D) were taken using a Nikon D7500 camera at 1/30 sec exposure. The same process was followed using HEK293 cells (see FIGS. 6C and 6D). As illustrated in FIGS. 6A and 6B, significant fluorescence was observed when the labelled aptamers were incubated with HNepC, while no fluorescence was detected with HEK293 cells, confirming the stronger binding affinity of the aptamers towards surface markers on the surface of HNepC (e.g. ICAM-1) compared to markers on HEK293 cells.

### B.2. Visualizing Virus Inhibition on H1-HeLa Cells by a Viral Inhibition Assay Using Fluorescence

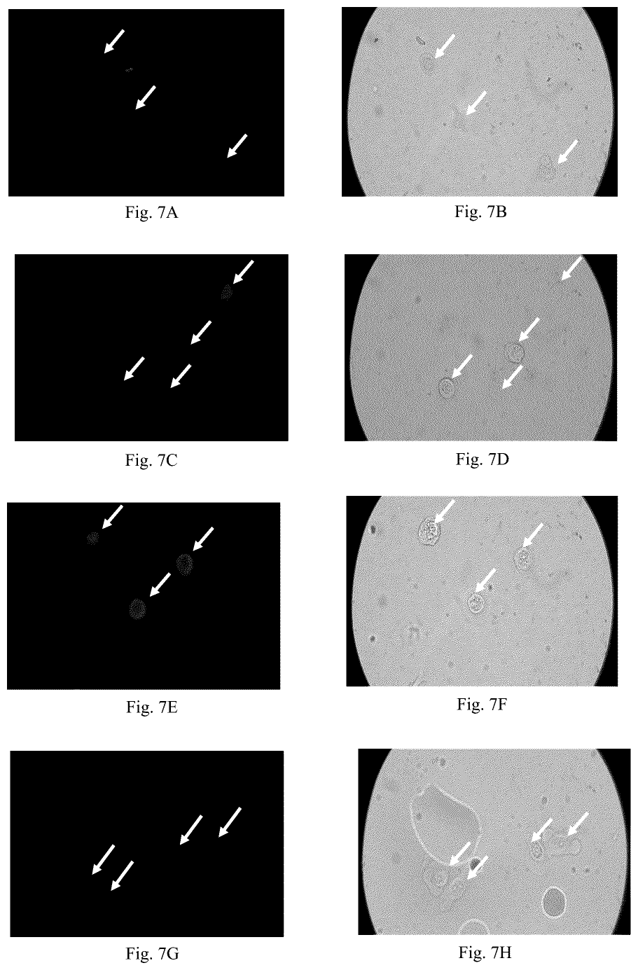

DNA aptamers Nas.R-2 and Nas.R-8 that bind to ICAM-1 were tested in a viral inhibition assay compared to a negative control aptamer to demonstrate their efficacy in blocking Rhinovirus infection (FIG. 7).

### B.2.1. Aptamer Incubation and Viral Infection

H1-HeLa cells in RPMI+2% Fetal Bovine Serum were seeded onto 24-well plates at 1×10⁵ cells/mL and 1.0 mL/well. The seed medium was aspirated, and 0.5 mL of each aptamer at 40 μM was added to the host cell wells. The host cells were incubated for 30±5 minutes at 33±2° C. with 5±3% CO₂. 0.5 mL of Rhinovirus Type 14 at 10³ TCID₅₀/well was added to the host cell wells without aspiration. The host cell wells were incubated 120±10 minutes at 33±2° C. with 5±3% CO₂. The host cells wells were aspirated and refed with 1.0 mL of each aptamer in cell culture medium and returned to incubation at 33±2° C. with 5±3% CO₂. After 18±1 hours, the cells were refed with 1.0 mL of a 2× concentration of aptamer in cell culture medium and incubated for 12±1 hours at 33±2° C. with 5±3% CO₂.

### B.2.2. Quantification of Viral Inhibition

After the total incubation period the host cell plates were frozen at −60 to −90° C. overnight and then thawed at ambient temperature. The contents of each well were individually harvested and centrifuged at 2,000 rpm for 10 minutes. The supernatant of each harvest was collected, serially diluted in cell culture medium and inoculated onto fresh H1-HeLa cells to determine the quantity of infectious virus using a Tissue Culture Infectious Does 50% (TCID₅₀) assay. The average yield of virus from control wells with cells treated with cell culture medium only were used to calculate the viral inhibitory activity (Log₁₀ reduction) by each aptamer.

TABLE 1
B.2.3.: Results

| | Log Viral | |
|---|---|---|
| Aptamer | Titer Reduction | Reduction (%) |
| Nas.R-2 | 2.08 | 99.2 |
| Nas.R-8 | 1.33 | 95.3 |

FIGS. 7A-7H show the result as images. Red labelled cells can be seen in the fluorescent image, if the TRITC-labelled virus was able to infect the cells. The position of the cells in the fluorescent images was marked with an arrow based on the corresponding position in the brightfield image. No infection can be seen using the Nas.R-2 aptamer (FIG. 7A fluorescent image; FIG. 7B brightfield image). Nearly no infection can be seen using the Nas.R-8 aptamer (FIG. 7C fluorescent image; FIG. 7D brightfield image). The cells were infected and appear red (FIG. 7E) using the negative control aptamer (FIG. 7F brightfield image) and FIGS. 7G and H show the control cells which were not infected with the virus (FIG. 7G fluorescent image FIG. 7H brightfield image).

### C. Determination of Binding Affinity by Surface Plasmon Resonance (SPR)

### C.1. Immobilization of RNA Aptamers in Gold Chips

RNA aptamers Nas.R-1, Nas.R-2, Nas.R-4, Nas.R-8, and a negative control were immobilized on the surface of gold chips. In brief, the RNA aptamer was dissolved in 1×PBS buffer supplemented with 10 mM EDTA. Then, an aliquot of 20 μL of this solution was added to 3.375 mg of 1-ethyl-3-(3-dimethylaminopropyl)carbodiimide (EDC) in a 1.5 mL tube. Next, an aliquot of 13.5 μL of cystamine-imidazole solution was added to the RNA aptamer and EDC solution, followed by mixing and centrifugation. The supernatant was removed and an additional aliquot of 54 μL of 100 μM imidazole (pH 6.0) was added. The solution was incubated at room temperature overnight. Finally, an RNA cleanup column was used to remove unincorporated cystamine and imidazole.

After conjugation of the cystamine moeities to phosphoramidate bonds at the 5′ phosphate group, the aptamer was immobilized on a gold chip by depositing an aliquot of 10 nL of aptamer solution at a concentration of 10 μM onto the surface of the chip. The gold reduces the cystamine to a pair of thiols and then catalyzes the reduction reaction that results in the covalent bond between the gold surface and the thiol groups of the modified aptamers.

### C.2. Surface Plasmon Resonance (SPR) Procedure

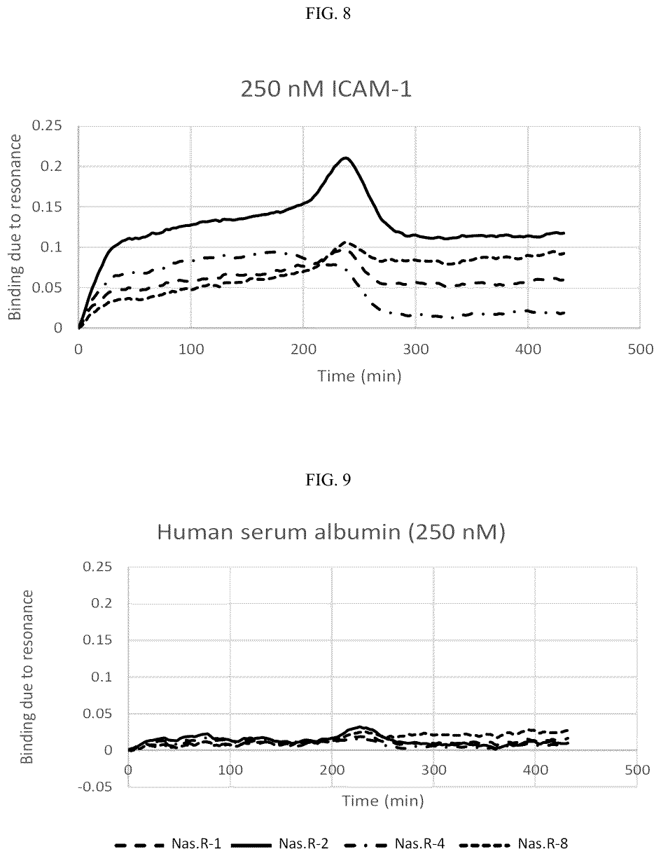

Solutions of 200 μL of ICAM-1 protein or human serum albumin were flown over the gold chip at a concentration of 250 nM and a flow rate of 50 μL/min using an Openplex Surface Plasmon Resonance System (Horiba, Kyoto, Kyoto, Japan). Thus, the association phase lasted for 4 minutes after injection and was immediately followed by the disassociation phase (see FIGS. 8 and 9). The total resonance of the negative control aptamer was subtracted from the total resonance observed for each of the candidate aptamers. The result corresponds to the resonance contribution due to the binding of the protein to the aptamer.

The kd (koff) value was calculated by fitting the curve to equation [1]:

$${x}^{′} ∼ - kd * x \qquad [ 1 ]$$

wherein x is the resonance due to binding and x′ is the derivative of this value at each time point captured on the disassociation curve. The kd value is then used to determine the ka value by using equation [2]:

x′˜ka*Rmax*c−(ka*c+kd)*x [2]

where Rmax is the maximum resonance due to binding observed, and c is the concentration of the injectant. Finally, the dissociation equilibrium constant kD was calculated as the ratio of kd over ka (see Table 2). The low nanomolar kD values obtained for the different aptamers confirm the strong binding affinity of such molecules towards ICAM-1 and validate the aptamer selection process described in Example 1. As used herein, “kd” refers to the dissociation rate, “ka” refers to the association rate, and “kD” refers to the dissociation equilibrium constant.

TABLE 2
Binding Coefficients of Nas.R-1, Nas.R-2, Nas.R-4,
and Nas.R-8 on 250 nM Exogenous ICAM-1.

| Aptamer | Nas.R-1 | Nas.R-2 | Nas.R-4 | Nas.R-8 |
|---|---|---|---|---|
| kd, [1/s] | 1.27E−02 | 1.42E−02 | 2.25E−02 | 2.63E−03 |
| ka, [1/M · s] | 1.97E+05 | 2.02E+05 | 5.08E+05 | 9.27E+04 |
| kD, [M] | 6.44E−08 | 7.02E−08 | 4.43E−08 | 2.84E−08 |

### D. Aptamer Binding Specificity

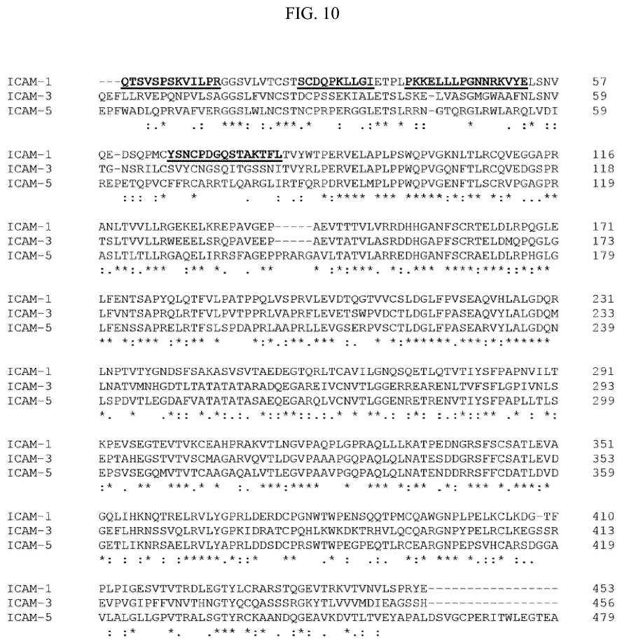

As described in Example 1, in the selection process, a counter selection was performed against with HEK293 cells. HEK293 cells do not express the ICAM-1 receptor, but they do express the related receptor proteins ICAM-3 and ICAM-5. For certain sequences, for instance Nas.R-2 (SEQ ID NO: 2), substantially higher affinity to nasal cells compared to HEK293 cells was observed. Not wishing to be bound by theory, given the presence of ICAM-5 and ICAM-3 on the HEK293 cells, it stands to reason that the selected aptamers are binding to epitopes from regions of the ICAM-1 receptor protein that are different in sequence from those of the ICAM-5 or ICAM-3 receptors. FIG. 10 illustrates the sequence alignment of ICAM-1, ICAM-3, and ICAM-5 and the regions that are likely to give rise to ICAM-1 specific binding are highlighted.

Rhinoviruses bind to the N-terminal Ig-like C2-type 1 domain of ICAM-1 receptor. Given the selection strategy, including elution with human rhinovirus particles, and counter selection against HEK293 cells, it is clear to one trained in the art that the mature selected aptamer library would be enriched in aptamer sequences that not only bind to the extracellular domain of the ICAM-1 receptor but do so specifically to the Ig-like C2-type 1 domain at the N-terminus.

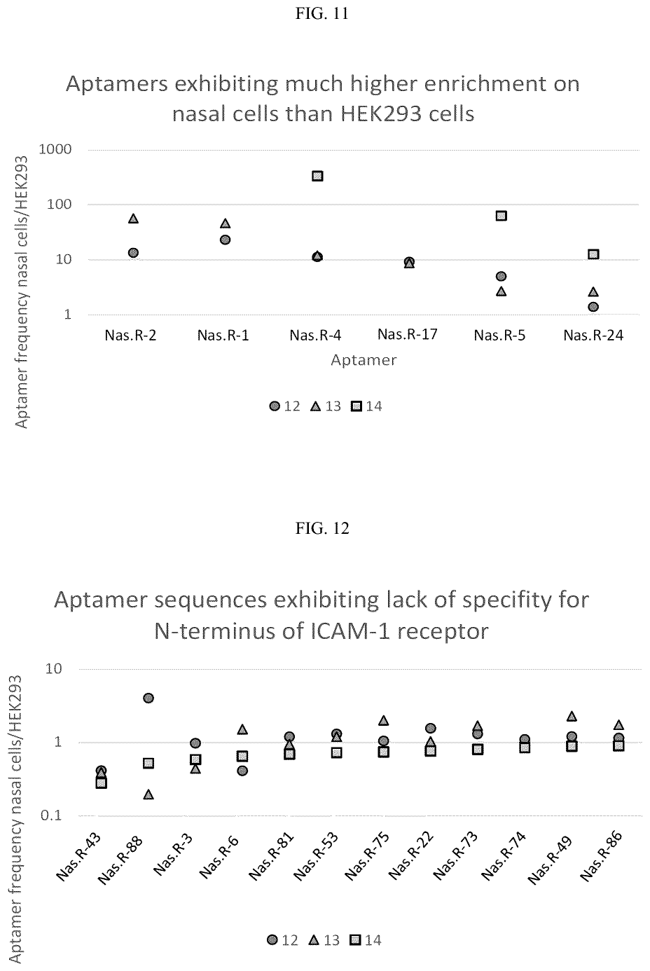

FIG. 11 illustrates a fold comparison in aptamer frequency over the final three selection rounds applied in the aptamer selection process. The data is presented as the frequency of the individual aptamer sequence as selected against nasal cells divided by the frequency of the same sequence observed in selection against HEK293 cells. For aptamers Nas.R-2, Nas.R-1, and Nas.R-17, the sequences were not observed in the selections against HEK293 cells (the legend refers to the selection round). That is, at least in terms of the subsample of sequences observed in the next generation sequencing process, these sequences were observed at high frequency in selection round 14 against the nasal cells but not observed at all in the selections against HEK293 cells.

Not wishing to be bound by theory, aptamers that did not exhibit enrichment in frequency when selected on nasal cells compared to HEK293 cells should be considered as aptamers that likely would not block HRV binding. FIG. 12 depicts sequences that in selection round 14 all exhibited higher enrichment levels with HEK293 positive selection than with positive selection against nasal cells. These aptamers would be expected to bind to regions of the ICAM-1 receptor that are not in the N-terminus and that have considerable sequence identity with regions of ICAM-3 or ICAM-5.

### Example 3. Analysis of Sequences Similarity

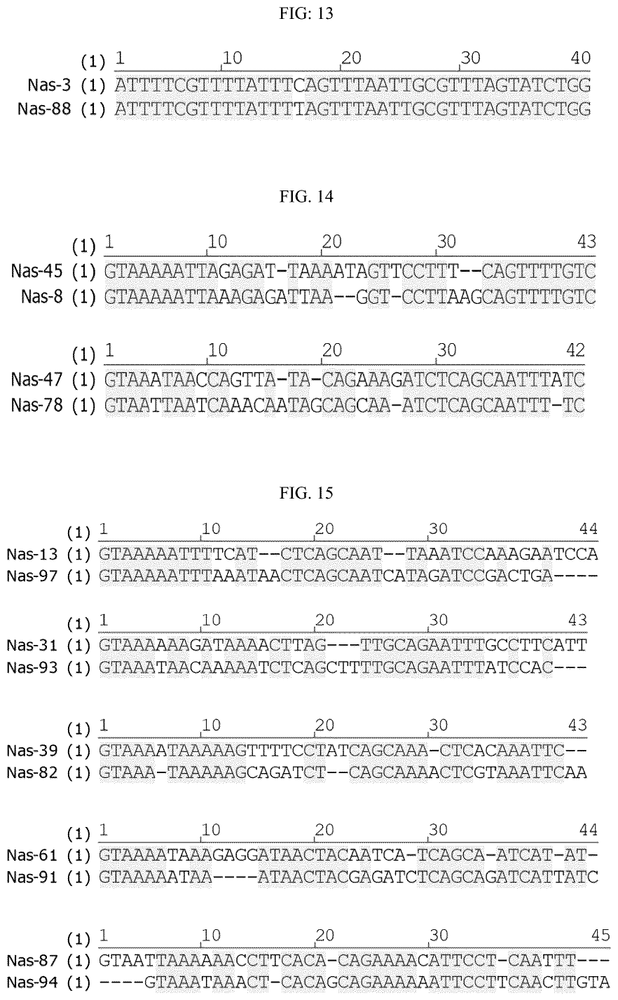

Alignment of SEQ ID NO: 1 to SEQ ID NO: 100 was performed using the software Align X, a component of Vector NTI Advanced 11.5.4 by Invitrogen. Several groups of sequences have at least 90%, at least 70%, or at least 50% nucleotide sequence identity as illustrated in the alignments of FIGS. 13, 14, and 15. In these alignments, only the central variable region of the aptamers was included for simplicity. Thus, oligonucleotides with at least 50%, at least 70%, or at least 90% nucleotide sequence identity to sequences selected from the group consisting of SEQ ID NO: 1 to SEQ ID NO: 200 are included as part of the current invention.

### Example 4. Motif Analysis and Predicted Secondary Structure

Aptamers bind to target molecules on the basis of the lowest free-energy shape that they form. The lowest free energy shape is a function of homology between regions within the single stranded sequence. These regions of homology fold back onto each other and thus create the secondary and tertiary shape of the aptamer that is crucial to enable binding. We characterized the core characteristics of these aptamers through a combined analysis of conserved motif sequences and their effect on the predicted structure of the whole aptamer. A motif in this context is defined as a contiguous sequence of nucleotides of a defined length. For this example, we considered each possible overlapping six nucleotide motif within the random region of each aptamer characterized.

The frequency of motifs of six nucleotides from the random regions of the top aptamers (Nas.R-1, Nas.R-2, Nas.R-4, and Nas.R-8) within all the sequences of selection round 14—Nasal Epithelial Cell Split library was determined. Then, the average motif frequency was subtracted from the frequency of each motif and this value was divided by the standard deviation of all the motifs frequencies in that selection round, resulting in a Z value for every motif. It stands to reason that sequences containing high frequency motifs also bind to the target molecule and are part of the present invention.

The prediction of the secondary structures of the aptamers was performed with The Vienna RNA Websuite. (http://rna.tbi.univie.ac.at//cgi-bin/RNAWebSuite/RNAfold.cgi. Gruber A R, Lorenz R, Bernhart S H, Neuböck R, Hofacker I L; Nucleic Acids Research, Volume 36, Issue suppl_2, 1 Jul. 2008, Pages W70-W74, DOI: 10.1093/nar/gkn188) and the motifs are highlighted within these structures.

A. Analysis of the Role of Conserved Motifs on Structure within the Aptamer Nas.R-1:

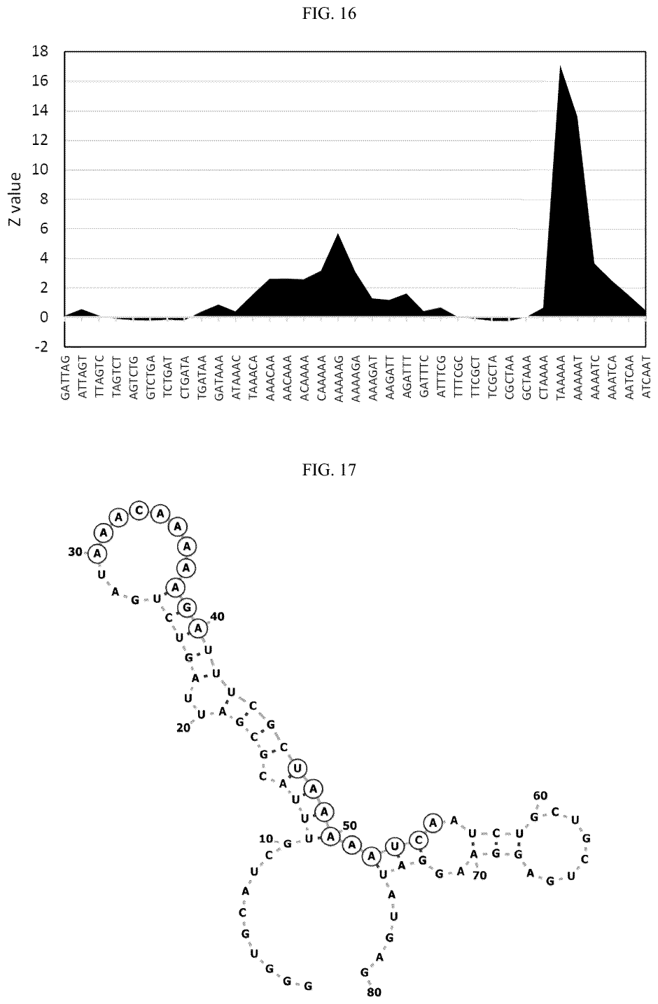

The results of motif analysis are presented in FIG. 16. The overlapping six nucleotide motifs comprising the random region of the aptamer are provided consecutively along the x axis in this figure. The y axis provides a statistical significance (Z value) for each motif in the library. The Z value was computed as the observed frequency of this motif in the library minus the average of the frequency for all motifs in the library and this subtractant was divided by the standard deviation of all motifs in the library to provide the Z value. Thus, a Z value of 2 represents a frequency of this motif in the library that is two standard deviations greater than the average value for all motifs.

In FIG. 16, it is clear that the sequences AAACAAAAAGA (see, e.g., SEQ ID NO: 201) and UAAAAAUCA (see, e.g., SEQ ID NO: 202) were conserved at a level that represented more than two standard deviations from the average. The lowest free energy predicted structure of the Nas.R-1 aptamer and the consensus sequences are shown in FIG. 17.

SEQ ID NO: 201:
5′-AAACAAAAAGA-3′
SEQ ID NO: 202:
5′-UAAAAAUCA-3′

Sequences containing any of these motifs are also expected to bind to ICAM-1 and are included as embodiments of the present invention. The conclusions arrived at within this example regarding conserved motifs in the RNA sequence would apply to the DNA sequence as well. Thus, any sequences containing the corresponding deoxyribonucleotide motif

SEQ ID NO: 203:
5′-AAACAAAAAGA-3′
SEQ ID NO: 204:
5′-TAAAAATCA-3′

are also included as embodiments.
B. Analysis of the Role of Conserved Motifs on Structure within the Aptamer Nas.R-4:

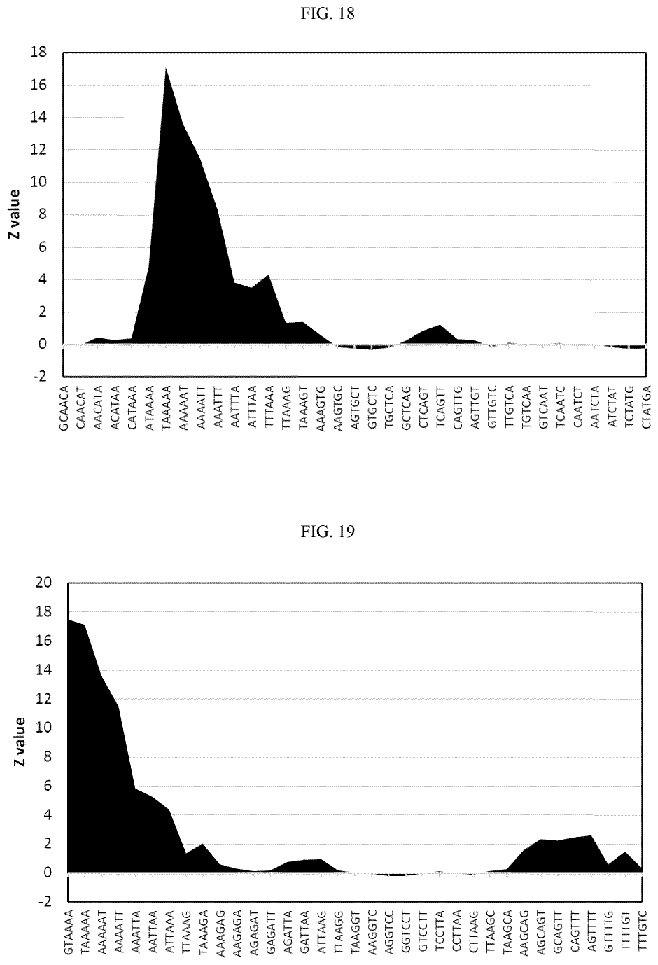

The analysis of the role of conserved motifs on structure within aptamer Nas.R-4 was performed in a manner identical to that described for Nas.R-1. FIG. 18 provides a summary of the motif analysis for aptamer Nas.R-4. There is a thirteen-nucleotide motif present at a frequency that was more than two standard deviations from the overall average motif frequency in the selected libraries,

SEQ ID NO 205:
5′-AUAAAAAUUUAAA-3′.

Sequences containing this motif are also expected to bind to ICAM-1 and are included as embodiments of the present invention. Any sequences containing the corresponding deoxyribonucleotide motif:

SEQ ID NO 206:
5′-ATAAAAATTTAAA-3′.

are also expected to bind to ICAM-1 and are included as embodiments of the present invention.
C. Analysis of the Role of Conserved Motifs on Structure within the Aptamer Nas.R-8:

The analysis of the role of conserved motifs on structure within aptamer Nas.R-8 was performed in a manner identical to that described for Nas.R-1 and Nas.R-4. FIG. 19 provides a summary of the motif analysis for aptamer Nas.R-8. There is a twelve-nucleotide motif present at a frequency that was more than two standard deviations from the overall average motif frequency in the selected libraries,

SEQ ID NO: 207:
5′-GUAAAAAUUAAA-3′

Sequences containing this motif are also expected to bind to ICAM-1 and are included as embodiments of the present invention. Furthermore, any sequences containing the corresponding deoxyribonucleotide motif:

SEQ ID NO 208:
5′-GTAAAAATTAAA-3′

are also expected to bind to ICAM-1 and are included as embodiments.
D. Analysis of Common Motifs within Aptamer Library:

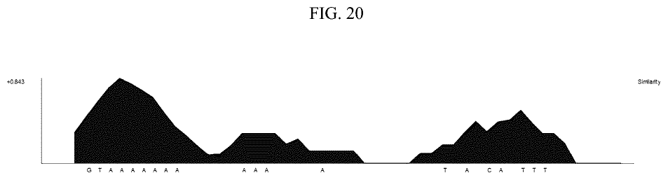

A search for common motifs within the top 100 sequences in terms of frequency was performed (see FIG. 20). The lead motifs identified in terms of significant deviation from random distribution were SEQ ID NO: 209 and SEQ IP NO: 210.

SEQ ID NO: 209:
5′-GUAAAAAAA-3′
SEQ ID NO: 210:
5′-UNAGCANUUU-3′

Oligonucleotides comprising the motifs SEQ ID NO: 209, SEQ ID NO: 210, or both are included as an embodiment of the current invention. Similarly, any sequences containing the corresponding deoxyribonucleotide motifs

SEQ ID NO: 211:
5′-GTAAAAAAA-3′
SEQ ID NO: 212:
5′-TNAGCANTTT-3′

are also expected to bind to ICAM-1 and are included as embodiments of the present invention.

TABLE 3
List of top sequences from selection
experiment. All the pyrimidine nucleotides
are fluorinated at the 2′ position of the
pentose group.

| SEQ | | |
|---|---|---|
| ID | | |
| NO | Name | Sequence |
| 1 | Nas.R-1 | GGGUGCAUCGUUUACGCGAUUAGUC |
| | | UGAUAAACAAAAAGAUUUCGCUAAA |
| | | AAUCAAUCUGCUGCUGAGGAAGGAU |
| | | AUGAG |
| 2 | Nas.R-2 | GGGUGCAUCGUUUACGCAGAUAGCA |
| | | GCAGGAAUCAAGCGGUAGGAGUCUA |
| | | GCAGAAGCUGCUGCUGAGGAAGGAU |
| | | AUGAG |
| 3 | Nas.R-3 | GGGUGCAUCGUUUACGCAUUUUCGU |
| | | UUUAUUUCAGUUUAAUUGCGUUUAG |
| | | UAUCUGGCUGCUGCUGAGGAAGGAU |
| | | AUGAG |
| 4 | Nas.R-4 | GGGUGCAUCGUUUACGCGCAACAUA |
| | | AAAAUUUAAAGUGCUCAGUUGUCAA |
| | | UCUAUGACUGCUGCUGAGGAAGGAU |
| | | AUGAG |
| 5 | Nas.R-5 | GGGUGCAUCGUUUACGCGUAAAUGG |
| | | UCCGCUAUUAAAAGAAAAGAAUGAA |
| | | GUCUCAGCUGCUGCUGAGGAAGGAU |
| | | AUGAG |
| 6 | Nas.R-6 | GGGUGCAUCGUUUACGCUAUUUUCA |
| | | UUUGUUUUUUUAAUUUACUAGUGUA |
| | | AACAAUCCUGCUGCUGAGGAAGGAU |
| | | AUGAG |
| 7 | Nas.R-7 | GGGUGCAUCGUUUACGCGUAAAUAA |
| | | GUAGAUAAAGUGGCAGUUUGUUUUC |
| | | CUUGGAACUGCUGCUGAGGAAGGAU |
| | | AUGAG |
| 8 | Nas.R-8 | GGGUGCAUCGUUUACGCGUAAAAAU |
| | | UAAAGAGAUUAAGGUCCUUAAGCAG |
| | | UUUUGUCCUGCUGCUGAGGAAGGAU |
| | | AUGAG |
| 9 | Nas.R-9 | GGGUGCAUCGUUUACGCGUAAAAAA |
| | | AUCAAAACUUCAGCAAAUUAUUUAU |
| | | CAACGUCCUGCUGCUGAGGAAGGAU |
| | | AUGAG |
| 10 | Nas.R-10 | GGGUGCAUCGUUUACGCGUAAAAUA |
| | | AAUUAAAAAGAACUUCUUCAGCAAU |
| | | CAAUAUCCUGCUGCUGAGGAAGGAU |
| | | AUGAG |
| 11 | Nas.R-11 | GGGUGCAUCGUUUACGCGUAAAUAA |
| | | AAAUGAAAAAUUGUCUCUCAGCUUU |
| | | CAAAGUCCUGCUGCUGAGGAAGGAU |
| | | AUGAG |
| 12 | Nas.R-12 | GGGUGCAUCGUUUACGCGUAAAAAA |
| | | AAAAUAUCUUCGGAGAAUUCAGCAA |
| | | UUUUAUCCUGCUGCUGAGGAAGGAU |
| | | AUGAG |
| 13 | Nas.R-13 | GGGUGCAUCGUUUACGCGUAAAAAU |
| | | UUUCAUCUCAGCAAUUAAAUCCAAA |
| | | GAAUCCACUGCUGCUGAGGAAGGAU |
| | | AUGAG |
| 14 | Nas.R-14 | GGGUGCAUCGUUUACGCGUAAAAUA |
| | | UAUCAGCAAAGUAGUUUAAGCCUCC |
| | | UCAGUUUCUGCUGCUGAGGAAGGAU |
| | | AUGAG |
| 15 | Nas.R-15 | GGGUGCAUCGUUUACGCGUAAAUUA |
| | | UGAAAAAUACAGCAAGGAUUUAACC |
| | | UCAGUUUCUGCUGCUGAGGAAGGAU |
| | | AUGAG |
| 16 | Nas.R-16 | GGGUGCAUCGUUUACGCGUAAAAUA |
| | | AAUAAAUCUUCAAAGUACAGACCUC |
| | | GAUUUUUCUGCUGCUGAGGAAGGAU |
| | | AUGAG |
| 17 | Nas.R-17 | GGGUGCAUCGUUUACGCUUAUAGGU |
| | | AUUAGACAUUUUCAAUUAAAGUGAA |
| | | UUAGUGUCUGCUGCUGAGGAAGGAU |
| | | AUGAG |
| 18 | Nas.R-18 | GGGUGCAUCGUUUACGCGUAAAAUG |
| | | UGACAGCAGGAUAAUAAAAUAAGUA |
| | | CUCAGUACUGCUGCUGAGGAAGGAU |
| | | AUGAG |
| 19 | Nas.R-19 | GGGUGCAUCGUUUACGCGUAAUUAA |
| | | GAAAAAUAAAAGUACUCUGCAGUUU |
| | | UUAUCCACUGCUGCUGAGGAAGGAU |
| | | AUGAG |
| 20 | Nas.R-20 | GGGUGCAUCGUUUACGCGUAAAAAU |
| | | AAAAUUUUCCCAGACCAGUUAUCUG |
| | | CCUUAAACUGCUGCUGAGGAAGGAU |
| | | AUGAG |
| 21 | Nas.R-21 | GGGUGCAUCGUUUACGCGUAAAGAA |
| | | AAAAAUCAGCUUUUAGUCGCCUUCC |
| | | AUUUUGACUGCUGCUGAGGAAGGAU |
| | | AUGAG |
| 22 | Nas.R-22 | GGGUGCAUCGUUUACGCGUAAAUAA |
| | | AUAAUCAAAAUUACACUCAGUGGCA |
| | | AUUUCCUCUGCUGCUGAGGAAGGAU |
| | | AUGAG |
| 23 | Nas.R-23 | GGGUGCAUCGUUUACGCGUAAAAUA |
| | | CAGGAUACGACAAUAACUCAGCAGA |
| | | UUUUAUCCUGCUGCUGAGGAAGGAU |
| | | AUGAG |
| 24 | Nas.R-24 | GGGUGCAUCGUUUACGCGUUAAAAA |
| | | UUGUGCACUGAGAUGACGCAGCAUU |
| | | AACUACACUGCUGCUGAGGAAGGAU |
| | | AUGAG |
| 25 | Nas.R-25 | GGGUGCAUCGUUUACGCGUAAAUAA |
| | | AAAUUAAUCAGCAAUUUUCCACUCA |
| | | GUUGUACCUGCUGCUGAGGAAGGAU |
| | | AUGAG |
| 26 | Nas.R-26 | GGGUGCAUCGUUUACGCGUAAAAAU |
| | | AAAAAAUCUCGAUCACUGCAGUUUU |
| | | AUUCCGGCUGCUGCUGAGGAAGGAU |
| | | AUGAG |
| 27 | Nas.R-27 | GGGUGCAUCGUUUACGCGUAAACAA |
| | | AUAUCGAUUAAAAUAAAAUCUCAGC |
| | | AAGAAUCCUGCUGCUGAGGAAGGAU |
| | | AUGAG |
| 28 | Nas.R-28 | GGGUGCAUCGUUUACGCGUAAAAUA |
| | | AAUAAAAUUAUCCCAGGAGCAAAUU |
| | | UUCUUCGCUGCUGCUGAGGAAGGAU |
| | | AUGAG |
| 29 | Nas.R-29 | GGGUGCAUCGUUUACGCGUAGAAGA |
| | | AUUAAUAGUGGACAUAUCAAUAGCA |
| | | GUUUAUCCUGCUGCUGAGGAAGGAU |
| | | AUGAG |
| 30 | Nas.R-30 | GGGUGCAUCGUUUACGCGUAAACAU |
| | | AUUCAGCAGUUAAAAUUUAGUAGGU |
| | | UCAGUAGCUGCUGCUGAGGAAGGAU |
| | | AUGAG |
| 31 | Nas.R-31 | GGGUGCAUCGUUUACGCGUAAAAAA |
| | | GAUAAAACUUAGUUGCAGAAUUUGC |
| | | CUUCAUUCUGCUGCUGAGGAAGGAU |
| | | AUGAG |
| 32 | Nas.R-32 | GGGUGCAUCGUUUACGCGUAAAAAG |
| | | UUUGAUGGAAGCAGAUUAGUUUAGU |
| | | CAAAUUUCUGCUGCUGAGGAAGGAU |
| | | AUGAG |
| 33 | Nas.R-33 | GGGUGCAUCGUUUACGCGUAAAAUG |
| | | AAAUAAGGAAUCCUUCAGCAGUAUU |
| | | UAUCCUUCUGCUGCUGAGGAAGGAU |
| | | AUGAG |
| 34 | Nas.R-34 | GGGUGCAUCGUUUACGCGUAAAGAA |
| | | UAAAAAUGACAAAAUUCUCAGCUUU |
| | | UGUCAACCUGCUGCUGAGGAAGGAU |
| | | AUGAG |
| 35 | Nas.R-35 | GGGUGCAUCGUUUACGCGUAAAAAA |
| | | UGAAAUGAAAAAAUUCUCAGCUGUC |
| | | UAUCUUCCUGCUGCUGAGGAAGGAU |
| | | AUGAG |
| 36 | Nas.R-36 | GGGUGCAUCGUUUACGCGUAAAUAA |
| | | GUAAAAAACUCAGUUUUCAGUUAAG |
| | | UAUCCAACUGCUGCUGAGGAAGGAU |
| | | AUGAG |
| 37 | Nas.R-37 | GGGUGCAUCGUUUACGCGUAAAUUU |
| | | CAGCAGAGUAAUAAUAACACUUCUU |
| | | CAGUUUGCUGCUGCUGAGGAAGGAU |
| | | AUGAG |
| 38 | Nas.R-38 | GGGUGCAUCGUUUACGCGUAAAAUU |
| | | AAGAAGUAUUAUCAGUUAGCUUUUU |
| | | CUUCCAACUGCUGCUGAGGAAGGAU |
| | | AUGAG |
| 39 | Nas.R-39 | GGGUGCAUCGUUUACGCGUAAAAUA |
| | | AAAAGUUUUCCUAUCAGCAAACUCA |
| | | CAAAUUCCUGCUGCUGAGGAAGGAU |
| | | AUGAG |
| 40 | Nas.R-40 | GGGUGCAUCGUUUACGCGUAAAAUG |
| | | AAAUGUAAAAGAAUUGAACUUGGCA |
| | | GAUUUUCCUGCUGCUGAGGAAGGAU |
| | | AUGAG |
| 41 | Nas.R-41 | GGGUGCAUCGUUUACGCGUAAAUUA |
| | | AAGUAGCAGUAAUUUCAGCAGUUUU |
| | | UACCUCUCUGCUGCUGAGGAAGGAU |
| | | AUGAG |
| 42 | Nas.R-42 | GGGUGCAUCGUUUACGCGUAAAUAA |
| | | AGGAUAAAAUAAUUUCAGGGCAGUU |
| | | UCUCAUCCUGCUGCUGAGGAAGGAU |
| | | AUGAG |
| 43 | Nas.R-43 | GGGUGCAUCGUUUACGCAGGAUCGU |
| | | UUUAAGUAAAAUAAAAGAUUUCCUU |
| | | GGUAAUCCUGCUGCUGAGGAAGGAU |
| | | AUGAG |
| 44 | Nas.R-44 | GGGUGCAUCGUUUACGCGUAAAAUA |
| | | AAGAUCAAUUAAAGGCUUUGAUCGA |
| | | UUUUCCUCUGCUGCUGAGGAAGGAU |
| | | AUGAG |
| 45 | Nas.R-45 | GGGUGCAUCGUUUACGCGUAAAAAU |
| | | UAGAGAUUAAAAUAGUUCCUUUCAG |
| | | UUUUGUCCUGCUGCUGAGGAAGGAU |
| | | AUGAG |
| 46 | Nas.R-46 | GGGUGCAUCGUUUACGCGUAAAAUU |
| | | GACAAUGUGAAAAGCAGACAGCAAA |
| | | UAUUCCUCUGCUGCUGAGGAAGGAU |
| | | AUGAG |
| 47 | Nas.R-47 | GGGUGCAUCGUUUACGCGUAAAUAA |
| | | CCAGUUAUACAGAAAGAUCUCAGCA |
| | | AUUUAUCCUGCUGCUGAGGAAGGAU |
| | | AUGAG |
| 48 | Nas.R-48 | GGGUGCAUCGUUUACGCUUACAGAA |
| | | GGAUUGCACCACAUGCGUACUCGAU |
| | | GAAACACCUGCUGCUGAGGAAGGAU |
| | | AUGAG |
| 49 | Nas.R-49 | GGGUGCAUCGUUUACGCGUAAAAUA |
| | | AUAAUUAAACUCAGCAAAUUCAAUC |
| | | CAACUUUCUGCUGCUGAGGAAGGAU |
| | | AUGAG |
| 50 | Nas.R-50 | GGGUGCAUCGUUUACGCGUAAACAA |
| | | GAAUAAAUUCAGCAGUGGUUUUGAU |
| | | CCUUUGACUGCUGCUGAGGAAGGAU |
| | | AUGAG |
| 51 | Nas.R-51 | GGGUGCAUCGUUUACGCGUAAAUUA |
| | | AUCAGAUUGAACAAAAGUUUUCCCU |
| | | CAGUUUUCUGCUGCUGAGGAAGGAU |
| | | AUGAG |
| 52 | Nas.R-52 | GGGUGCAUCGUUUACGCGUAAAGAA |
| | | AAACAUCAGAGCAGUUAUAAUAGUC |
| | | CUUUUUCCUGCUGCUGAGGAAGGAU |
| | | AUGAG |
| 53 | Nas.R-53 | GGGUGCAUCGUUUACGCGUAAAGAA |
| | | AAUAAACUUGAUCAAACUUAGCAGU |
| | | UUUUAUCCUGCUGCUGAGGAAGGAU |
| | | AUGAG |
| 54 | Nas.R-54 | GGGUGCAUCGUUUACGCAUUUUCGU |
| | | UAUAUUUCUGGUUUUUAUGCGUGAG |
| | | AAUCCUGCUGCUGCUGAGGAAGGAU |
| | | AUGAG |
| 55 | Nas.R-55 | GGGUGCAUCGUUUACGCGUAAAAAU |
| | | AAGAUCUCACAGCGACAAAUUUUUC |
| | | UUCCAGUCUGCUGCUGAGGAAGGAU |
| | | AUGAG |
| 56 | Nas.R-56 | GGGUGCAUCGUUUACGCGUAAAUUU |
| | | AAGACAUGACAGCAGACAUUUUAUC |
| | | UUCAGACCUGCUGCUGAGGAAGGAU |
| | | AUGAG |
| 57 | Nas.R-57 | GGGUGCAUCGUUUACGCGUAAUAAC |
| | | AGAAAUAUAACUCAGCUGAAUUAAU |
| | | UUUUCCGCUGCUGCUGAGGAAGGAU |
| | | AUGAG |
| 58 | Nas.R-58 | GGGUGCAUCGUUUACGCGUAAAAAU |
| | | AAAUUCCAAAAUAUUCAGCAGAAAU |
| | | CCUCGAACUGCUGCUGAGGAAGGAU |
| | | AUGAG |
| 59 | Nas.R-59 | GGGUGCAUCGUUUACGCGUAAAAAU |
| | | AAUAGGUUCCAAUCAAGCAGUACAA |
| | | AAUUCCUCUGCUGCUGAGGAAGGAU |
| | | AUGAG |
| 60 | Nas.R-60 | GGGUGCAUCGUUUACGCGUAAAAAA |
| | | UCUAAAAAGAUAUCAGCAGGCAAAU |
| | | UUUCCUUCUGCUGCUGAGGAAGGAU |
| | | AUGAG |
| 61 | Nas.R-61 | GGGUGCAUCGUUUACGCGUAAAAUA |
| | | AAGAGGAUAACUACAAUCAUCAGCA |
| | | AUCAUAUCUGCUGCUGAGGAAGGAU |
| | | AUGAG |
| 62 | Nas.R-62 | GGGUGCAUCGUUUACGCGUAAAUUU |
| | | AGUAGAAAGGAAAGACGAAGUUUCC |
| | | UCAGUUUCUGCUGCUGAGGAAGGAU |
| | | AUGAG |
| 63 | Nas.R-63 | GGGUGCAUCGUUUACGCGUAAAAAU |
| | | AAUAGAUCUCAGAAUAUGAAAGCAG |
| | | UUCUUUCCUGCUGCUGAGGAAGGAU |
| | | AUGAG |
| 64 | Nas.R-64 | GGGUGCAUCGUUUACGCGUAACAAG |
| | | AUAUUCACAGCAGAUUUUAAAAAAU |
| | | UCCUCGUCUGCUGCUGAGGAAGGAU |
| | | AUGAG |
| 65 | Nas.R-65 | GGGUGCAUCGUUUACGCGUAAAAAG |
| | | UUGACAAUUAAUAAAAUCUUCUUAG |
| | | CAUUUUCCUGCUGCUGAGGAAGGAU |
| | | AUGAG |
| 66 | Nas.R-66 | GGGUGCAUCGUUUACGCGUAAAACA |
| | | AAAUGAAACUUAUAGCUCAGCAUAU |
| | | UUUGAUCCUGCUGCUGAGGAAGGAU |
| | | AUGAG |
| 67 | Nas.R-67 | GGGUGCAUCGUUUACGCGUAAAUUA |
| | | UCAAAAAAGCAGAUUUAAGUAUACC |
| | | UCAGUUACUGCUGCUGAGGAAGGAU |
| | | AUGAG |
| 68 | Nas.R-68 | GGGUGCAUCGUUUACGCGUAAAUAA |
| | | AAUAGCUCAGCAAGGAAGUUUUUUU |
| | | CCUCAAACUGCUGCUGAGGAAGGAU |
| | | AUGAG |
| 69 | Nas.R-69 | GGGUGCAUCGUUUACGCGUAAAUUU |
| | | GAGAAAAGAACAGCAGACUCAAAUC |
| | | UUUUUAACUGCUGCUGAGGAAGGAU |
| | | AUGAG |
| 70 | Nas.R-70 | GGGUGCAUCGUUUACGCGUAACAGA |
| | | AAAUUAAGCUCAGCAAUAGUAAUUA |
| | | UCCUAGUCUGCUGCUGAGGAAGGAU |
| | | AUGAG |
| 71 | Nas.R-71 | GGGUGCAUCGUUUACGCGUAAUGAA |
| | | AAUAAAUCAGUCUCACAGCAUUUUA |
| | | AAACUUCCUGCUGCUGAGGAAGGAU |
| | | AUGAG |
| 72 | Nas.R-72 | GGGUGCAUCGUUUACGCGUAUUUAC |
| | | AAGCAACAAAGUUACAAUCAGCAGA |
| | | AUUUAUCCUGCUGCUGAGGAAGGAU |
| | | AUGAG |
| 73 | Nas.R-73 | GGGUGCAUCGUUUACGCGUAAAAAA |
| | | UUGUCUAUAGCACUUUUAGAUUCCC |
| | | AAACUAACUGCUGCUGAGGAAGGAU |
| | | AUGAG |
| 74 | Nas.R-74 | GGGUGCAUCGUUUACGCGUAAAAAA |
| | | AUCAGCAAAAUCGAAAACUCAUGCA |
| | | GUUUGUCCUGCUGCUGAGGAAGGAU |
| | | AUGAG |
| 75 | Nas.R-75 | GGGUGCAUCGUUUACGCGUAAAAAA |
| | | UUCCUUAAAAAUUUAACUAACUGGA |
| | | UAGGUCUCUGCUGCUGAGGAAGGAU |
| | | AUGAG |
| 76 | Nas.R-76 | GGGUGCAUCGUUUACGCGUAAAACA |
| | | AAAUUUCUGACAGCAAUUCCUUCGU |
| | | UAAAAAUCUGCUGCUGAGGAAGGAU |
| | | AUGAG |
| 77 | Nas.R-77 | GGGUGCAUCGUUUACGCGUAAAUUA |
| | | UUAAAAAAAUCAGCAAAGUUUAUUU |
| | | CCCACGGCUGCUGCUGAGGAAGGAU |
| | | AUGAG |
| 78 | Nas.R-78 | GGGUGCAUCGUUUACGCGUAAUUAA |
| | | UCAAACAAUAGCAGCAAAUCUCAGC |
| | | AAUUUUCCUGCUGCUGAGGAAGGAU |
| | | AUGAG |
| 79 | Nas.R-79 | GGGUGCAUCGUUUACGCGUAAUUUG |
| | | AAAGUCUCAUAAAUUUUUUUUUUUU |
| | | UUUCAAUCUGCUGCUGAGGAAGGAU |
| | | AUGAG |
| 80 | Nas.R-80 | GGGUGCAUCGUUUACGCGUAAAAAU |
| | | UCAGCAUGAUUUCAAUUACUCCUUU |
| | | CAUUGAUCUGCUGCUGAGGAAGGAU |
| | | AUGAG |
| 81 | Nas.R-81 | GGGUGCAUCGUUUACGCGUAAAAUA |
| | | AAUAAAAAUCAGUAGCAAUCUUUCU |
| | | CACAGUGCUGCUGCUGAGGAAGGAU |
| | | AUGAG |
| 82 | Nas.R-82 | GGGUGCAUCGUUUACGCGUAAAUAA |
| | | AAAGCAGAUCUCAGCAAAACUCGUA |
| | | AAUUCAACUGCUGCUGAGGAAGGAU |
| | | AUGAG |
| 83 | Nas.R-83 | GGGUGCAUCGUUUACGCGUAAAUAA |
| | | UGAAGGACUCAGACAGUUAAAAGAU |
| | | GCAUUAACUGCUGCUGAGGAAGGAU |
| | | AUGAG |
| 84 | Nas.R-84 | GGGUGCAUCGUUUACGCGUAAAAAA |
| | | GAUCAAUAUGAAAAUCAGCAGUUAA |
| | | UAUCUUCCUGCUGCUGAGGAAGGAU |
| | | AUGAG |
| 85 | Nas.R-85 | GGGUGCAUCGUUUACGCGUAAAAAU |
| | | AACAAACUUCUCAGCUGUUUAAUAU |
| | | CUCCUGACUGCUGCUGAGGAAGGAU |
| | | AUGAG |
| 86 | Nas.R-86 | GGGUGCAUCGUUUACGCGUAAAAUU |
| | | AAACAAAUAGCUCAGCACGAAAAUU |
| | | UGCGUAACUGCUGCUGAGGAAGGAU |
| | | AUGAG |
| 87 | Nas.R-87 | GGGUGCAUCGUUUACGCGUAAUUAA |
| | | AAAACCUUCACACAGAAAACAUUCC |
| | | UCAAUUUCUGCUGCUGAGGAAGGAU |
| | | AUGAG |
| 88 | Nas.R-88 | GGGUGCAUCGUUUACGCAUUUUCGU |
| | | UUUAUUUUAGUUUAAUUGCGUUUAG |
| | | UAUCUGGCUGCUGCUGAGGAAGGAU |
| | | AUGAG |
| 89 | Nas.R-89 | GGGUGCAUCGUUUACGCGUAAAAAG |
| | | UAUAAAGGUUAGAAAUUCAGCAGUU |
| | | UGAUAUCCUGCUGCUGAGGAAGGAU |
| | | AUGAG |
| 90 | Nas.R-90 | GGGUGCAUCGUUUACGCGUAAAAAG |
| | | GAGAAUUAGUACUCACCAGUCGUUU |
| | | AAAAUUUCUGCUGCUGAGGAAGGAU |
| | | AUGAG |
| 91 | Nas.R-91 | GGGUGCAUCGUUUACGCGUAAAAAU |
| | | AAAUAACUACGAGAUCUCAGCAGAU |
| | | CAUUAUCCUGCUGCUGAGGAAGGAU |
| | | AUGAG |
| 92 | Nas.R-92 | GGGUGCAUCGUUUACGCGUAAAAUG |
| | | GUUUUUCAGCAGUUAACAUAAUGCC |
| | | UCAGUUUCUGCUGCUGAGGAAGGAU |
| | | AUGAG |
| 93 | Nas.R-93 | GGGUGCAUCGUUUACGCGUAAAUAA |
| | | CAAAAAUCUCAGCUUUUGCAGAAUU |
| | | UAUCCACCUGCUGCUGAGGAAGGAU |
| | | AUGAG |
| 94 | Nas.R-94 | GGGUGCAUCGUUUACGCGUAAAUAA |
| | | ACUCACAGCAGAAAAAAUUCCUUCA |
| | | ACUUGUACUGCUGCUGAGGAAGGAU |
| | | AUGAG |
| 95 | Nas.R-95 | GGGUGCAUCGUUUACGCAGUAGUUA |
| | | AUAACAAAUAGUCAGCAGUUUUGUC |
| | | CUUCAUUCUGCUGCUGAGGAAGGAU |
| | | AUGAG |
| 96 | Nas.R-96 | GGGUGCAUCGUUUACGCGUAAAAAU |
| | | AGCAGUAGAUAGCGGCAGUUUUGUA |
| | | UUUGUUACUGCUGCUGAGGAAGGAU |
| | | AUGAG |
| 97 | Nas.R-97 | GGGUGCAUCGUUUACGCGUAAAAAU |
| | | UUAAAUAACUCAGCAAUCAUAGAUC |
| | | CGACUGACUGCUGCUGAGGAAGGAU |
| | | AUGAG |
| 98 | Nas.R-98 | GGGUGCAUCGUUUACGCGUAAAGAA |
| | | CAGCUGACAAGAAAUUCAAACCUUC |
| | | AGAUUUUCUGCUGCUGAGGAAGGAU |
| | | AUGAG |
| 99 | Nas.R-99 | GGGUGCAUCGUUUACGCGUAAAGAU |
| | | AAUAAGCAGUAUUCAGCAGAUUUGU |
| | | AAGGUUUCUGCUGCUGAGGAAGGAU |
| | | AUGAG |
| 100 | Nas.R-100 | GGGUGCAUCGUUUACGCGUAAAUAA |
| | | GAGGCAGACAGUAUUACAAAUAUCC |
| | | UAAAAUACUGCUGCUGAGGAAGGAU |
| | | AUGAG |

TABLE 4
List of deoxyribonucleotides aptamers
based on the top sequences from
selection experiments.

| SEQ | | |
|---|---|---|
| ID NO | Name | Sequence |
| 101 | Nas.D-1 | GGGTGCATCGTTTACGCGAT |
| | | TAGTCTGATAAACAAAAAGA |
| | | TTTCGCTAAAAATCAATCTG |
| | | CTGCTGAGGAAGGATATGAG |
| 102 | Nas.D-2 | GGGTGCATCGTTTACGCAGA |
| | | TAGCAGCAGGAATCAAGCGG |
| | | TAGGAGTCTAGCAGAAGCTG |
| | | CTGCTGAGGAAGGATATGAG |
| 103 | Nas.D-3 | GGGTGCATCGTTTACGCATT |
| | | TTCGTTTTATTTCAGTTTAA |
| | | TTGCGTTTAGTATCTGGCTG |
| | | CTGCTGAGGAAGGATATGAG |
| 104 | Nas.D-4 | GGGTGCATCGTTTACGCGCA |
| | | ACATAAAAATTTAAAGTGCT |
| | | CAGTTGTCAATCTATGACTG |
| | | CTGCTGAGGAAGGATATGAG |
| 105 | Nas.D-5 | GGGTGCATCGTTTACGCGTA |
| | | AATGGTCCGCTATTAAAAGA |
| | | AAAGAATGAAGTCTCAGCTG |
| | | CTGCTGAGGAAGGATATGAG |
| 106 | Nas.D-6 | GGGTGCATCGTTTACGCTAT |
| | | TTTCATTTGTTTTTTTAATT |
| | | TACTAGTGTAAACAATCCTG |
| | | CTGCTGAGGAAGGATATGAG |
| 107 | Nas.D-7 | GGGTGCATCGTTTACGCGTA |
| | | AATAAGTAGATAAAGTGGCA |
| | | GTTTGTTTTCCTTGGAACTG |
| | | CTGCTGAGGAAGGATATGAG |
| 108 | Nas.D-8 | GGGTGCATCGTTTACGCGTA |
| | | AAAATTAAAGAGATTAAGGT |
| | | CCTTAAGCAGTTTTGTCCTG |
| | | CTGCTGAGGAAGGATATGAG |
| 109 | Nas.D-9 | GGGTGCATCGTTTACGCGTA |
| | | AAAAAATCAAAACTTCAGCA |
| | | AATTATTTATCAACGTCCTG |
| | | CTGCTGAGGAAGGATATGAG |
| 110 | Nas.D-10 | GGGTGCATCGTTTACGCGTA |
| | | AAATAAATTAAAAAGAACTT |
| | | CTTCAGCAATCAATATCCTG |
| | | CTGCTGAGGAAGGATATGAG |
| 111 | Nas.D-11 | GGGTGCATCGTTTACGCGTA |
| | | AATAAAAATGAAAAATTGTC |
| | | TCTCAGCTTTCAAAGTCCTG |
| | | CTGCTGAGGAAGGATATGAG |
| 112 | Nas.D-12 | GGGTGCATCGTTTACGCGTA |
| | | AAAAAAAAATATCTTCGGAG |
| | | AATTCAGCAATTTTATCCTG |
| | | CTGCTGAGGAAGGATATGAG |
| 113 | Nas.D-13 | GGGTGCATCGTTTACGCGTA |
| | | AAAATTTTCATCTCAGCAAT |
| | | TAAATCCAAAGAATCCACTG |
| | | CTGCTGAGGAAGGATATGAG |
| 114 | Nas.D-14 | GGGTGCATCGTTTACGCGTA |
| | | AAATATATCAGCAAAGTAGT |
| | | TTAAGCCTCCTCAGTTTCTG |
| | | CTGCTGAGGAAGGATATGAG |
| 115 | Nas.D-15 | GGGTGCATCGTTTACGCGTA |
| | | AATTATGAAAAATACAGCAA |
| | | GGATTTAACCTCAGTTTCTG |
| | | CTGCTGAGGAAGGATATGAG |
| 116 | Nas.D-16 | GGGTGCATCGTTTACGCGTA |
| | | AAATAAATAAATCTTCAAAG |
| | | TACAGACCTCGATTTTTCTG |
| | | CTGCTGAGGAAGGATATGAG |
| 117 | Nas.D-17 | GGGTGCATCGTTTACGCTTA |
| | | TAGGTATTAGACATTTTCAA |
| | | TTAAAGTGAATTAGTGTCTG |
| | | CTGCTGAGGAAGGATATGAG |
| 118 | Nas.D-18 | GGGTGCATCGTTTACGCGTA |
| | | AAATGTGACAGCAGGATAAT |
| | | AAAATAAGTACTCAGTACTG |
| | | CTGCTGAGGAAGGATATGAG |
| 119 | Nas.D-19 | GGGTGCATCGTTTACGCGTA |
| | | ATTAAGAAAAATAAAAGTAC |
| | | TCTGCAGTTTTTATCCACTG |
| | | CTGCTGAGGAAGGATATGAG |
| 120 | Nas.D-20 | GGGTGCATCGTTTACGCGTA |
| | | AAAATAAAATTTTCCCAGAC |
| | | CAGTTATCTGCCTTAAACTG |
| | | CTGCTGAGGAAGGATATGAG |
| 121 | Nas.D-21 | GGGTGCATCGTTTACGCGTA |
| | | AAGAAAAAAATCAGCTTTTA |
| | | GTCGCCTTCCATTTTGACTG |
| | | CTGCTGAGGAAGGATATGAG |
| 122 | Nas.D-22 | GGGTGCATCGTTTACGCGTA |
| | | AATAAATAATCAAAATTACA |
| | | CTCAGTGGCAATTTCCTCTG |
| | | CTGCTGAGGAAGGATATGAG |
| 123 | Nas.D-23 | GGGTGCATCGTTTACGCGTA |
| | | AAATACAGGATACGACAATA |
| | | ACTCAGCAGATTTTATCCTG |
| | | CTGCTGAGGAAGGATATGAG |
| 124 | Nas.D-24 | GGGTGCATCGTTTACGCGTT |
| | | AAAAATTGTGCACTGAGATG |
| | | ACGCAGCATTAACTACACTG |
| | | CTGCTGAGGAAGGATATGAG |
| 125 | Nas.D-25 | GGGTGCATCGTTTACGCGTA |
| | | AATAAAAATTAATCAGCAAT |
| | | TTTCCACTCAGTTGTACCTG |
| | | CTGCTGAGGAAGGATATGAG |
| 126 | Nas.D-26 | GGGTGCATCGTTTACGCGTA |
| | | AAAATAAAAAATCTCGATCA |
| | | CTGCAGTTTTATTCCGGCTG |
| | | CTGCTGAGGAAGGATATGAG |
| 127 | Nas.D-27 | GGGTGCATCGTTTACGCGTA |
| | | AACAAATATCGATTAAAATA |
| | | AAATCTCAGCAAGAATCCTG |
| | | CTGCTGAGGAAGGATATGAG |
| 128 | Nas.D-28 | GGGTGCATCGTTTACGCGTA |
| | | AAATAAATAAAATTATCCCA |
| | | GGAGCAAATTTTCTTCGCTG |
| | | CTGCTGAGGAAGGATATGAG |
| 129 | Nas.D-29 | GGGTGCATCGTTTACGCGTA |
| | | GAAGAATTAATAGTGGACAT |
| | | ATCAATAGCAGTTTATCCTG |
| | | CTGCTGAGGAAGGATATGAG |
| 130 | Nas.D-30 | GGGTGCATCGTTTACGCGTA |
| | | AACATATTCAGCAGTTAAAA |
| | | TTTAGTAGGTTCAGTAGCTG |
| | | CTGCTGAGGAAGGATATGAG |
| 131 | Nas.D-31 | GGGTGCATCGTTTACGCGTA |
| | | AAAAAGATAAAACTTAGTTG |
| | | CAGAATTTGCCTTCATTCTG |
| | | CTGCTGAGGAAGGATATGAG |
| 132 | Nas.D-32 | GGGTGCATCGTTTACGCGTA |
| | | AAAAGTTTGATGGAAGCAGA |
| | | TTAGTTTAGTCAAATTTCTG |
| | | CTGCTGAGGAAGGATATGAG |
| 133 | Nas.D-33 | GGGTGCATCGTTTACGCGTA |
| | | AAATGAAATAAGGAATCCTT |
| | | CAGCAGTATTTATCCTTCTG |
| | | CTGCTGAGGAAGGATATGAG |
| 134 | Nas.D-34 | GGGTGCATCGTTTACGCGTA |
| | | AAGAATAAAAATGACAAAAT |
| | | TCTCAGCTTTTGTCAACCTG |
| | | CTGCTGAGGAAGGATATGAG |
| 135 | Nas.D-35 | GGGTGCATCGTTTACGCGTA |
| | | AAAAATGAAATGAAAAAATT |
| | | CTCAGCTGTCTATCTTCCTG |
| | | CTGCTGAGGAAGGATATGAG |
| 136 | Nas.D-36 | GGGTGCATCGTTTACGCGTA |
| | | AATAAGTAAAAAACTCAGTT |
| | | TTCAGTTAAGTATCCAACTG |
| | | CTGCTGAGGAAGGATATGAG |
| 137 | Nas.D-37 | GGGTGCATCGTTTACGCGTA |
| | | AATTTCAGCAGAGTAATAAT |
| | | AACACTTCTTCAGTTTGCTG |
| | | CTGCTGAGGAAGGATATGAG |
| 138 | Nas.D-38 | GGGTGCATCGTTTACGCGTA |
| | | AAATTAAGAAGTATTATCAG |
| | | TTAGCTTTTTCTTCCAACTG |
| | | CTGCTGAGGAAGGATATGAG |
| 139 | Nas.D-39 | GGGTGCATCGTTTACGCGTA |
| | | AAATAAAAAGTTTTCCTATC |
| | | AGCAAACTCACAAATTCCTG |
| | | CTGCTGAGGAAGGATATGAG |
| 140 | Nas.D-40 | GGGTGCATCGTTTACGCGTA |
| | | AAATGAAATGTAAAAGAATT |
| | | GAACTTGGCAGATTTTCCTG |
| | | CTGCTGAGGAAGGATATGAG |
| 141 | Nas.D-41 | GGGTGCATCGTTTACGCGTA |
| | | AATTAAAGTAGCAGTAATTT |
| | | CAGCAGTTTTTACCTCTCTG |
| | | CTGCTGAGGAAGGATATGAG |
| 142 | Nas.D-42 | GGGTGCATCGTTTACGCGTA |
| | | AATAAAGGATAAAATAATTT |
| | | CAGGGCAGTTTCTCATCCTG |
| | | CTGCTGAGGAAGGATATGAG |
| 143 | Nas.D-43 | GGGTGCATCGTTTACGCAGG |
| | | ATCGTTTTAAGTAAAATAAA |
| | | AGATTTCCTTGGTAATCCTG |
| | | CTGCTGAGGAAGGATATGAG |
| 144 | Nas.D-44 | GGGTGCATCGTTTACGCGTA |
| | | AAATAAAGATCAATTAAAGG |
| | | CTTTGATCGATTTTCCTCTG |
| | | CTGCTGAGGAAGGATATGAG |
| 145 | Nas.D-45 | GGGTGCATCGTTTACGCGTA |
| | | AAAATTAGAGATTAAAATAG |
| | | TTCCTTTCAGTTTTGTCCTG |
| | | CTGCTGAGGAAGGATATGAG |
| 146 | Nas.D-46 | GGGTGCATCGTTTACGCGTA |
| | | AAATTGACAATGTGAAAAGC |
| | | AGACAGCAAATATTCCTCTG |
| | | CTGCTGAGGAAGGATATGAG |
| 147 | Nas.D-47 | GGGTGCATCGTTTACGCGTA |
| | | AATAACCAGTTATACAGAAA |
| | | GATCTCAGCAATTTATCCTG |
| | | CTGCTGAGGAAGGATATGAG |
| 148 | Nas.D-48 | GGGTGCATCGTTTACGCTTA |
| | | CAGAAGGATTGCACCACATG |
| | | CGTACTCGATGAAACACCTG |
| | | CTGCTGAGGAAGGATATGAG |
| 149 | Nas.D-49 | GGGTGCATCGTTTACGCGTA |
| | | AAATAATAATTAAACTCAGC |
| | | AAATTCAATCCAACTTTCTG |
| | | CTGCTGAGGAAGGATATGAG |
| 150 | Nas.D-50 | GGGTGCATCGTTTACGCGTA |
| | | AACAAGAATAAATTCAGCAG |
| | | TGGTTTTGATCCTTTGACTG |
| | | CTGCTGAGGAAGGATATGAG |
| 151 | Nas.D-51 | GGGTGCATCGTTTACGCGTA |
| | | AATTAATCAGATTGAACAAA |
| | | AGTTTTCCCTCAGTTTTCTG |
| | | CTGCTGAGGAAGGATATGAG |
| 152 | Nas.D-52 | GGGTGCATCGTTTACGCGTA |
| | | AAGAAAAACATCAGAGCAGT |
| | | TATAATAGTCCTTTTTCCTG |
| | | CTGCTGAGGAAGGATATGAG |
| 153 | Nas.D-53 | GGGTGCATCGTTTACGCGTA |
| | | AAGAAAATAAACTTGATCAA |
| | | ACTTAGCAGTTTTTATCCTG |
| | | CTGCTGAGGAAGGATATGAG |
| 154 | Nas.D-54 | GGGTGCATCGTTTACGCATT |
| | | TTCGTTATATTTCTGGTTTT |
| | | TATGCGTGAGAATCCTGCTG |
| | | CTGCTGAGGAAGGATATGAG |
| 155 | Nas.D-55 | GGGTGCATCGTTTACGCGTA |
| | | AAAATAAGATCTCACAGCGA |
| | | CAAATTTTTCTTCCAGTCTG |
| | | CTGCTGAGGAAGGATATGAG |
| 156 | Nas.D-56 | GGGTGCATCGTTTACGCGTA |
| | | AATTTAAGACATGACAGCAG |
| | | ACATTTTATCTTCAGACCTG |
| | | CTGCTGAGGAAGGATATGAG |
| 157 | Nas.D-57 | GGGTGCATCGTTTACGCGTA |
| | | ATAACAGAAATATAACTCAG |
| | | CTGAATTAATTTTTCCGCTG |
| | | CTGCTGAGGAAGGATATGAG |
| 158 | Nas.D-58 | GGGTGCATCGTTTACGCGTA |
| | | AAAATAAATTCCAAAATATT |
| | | CAGCAGAAATCCTCGAACTG |
| | | CTGCTGAGGAAGGATATGAG |
| 159 | Nas.D-59 | GGGTGCATCGTTTACGCGTA |
| | | AAAATAATAGGTTCCAATCA |
| | | AGCAGTACAAAATTCCTCTG |
| | | CTGCTGAGGAAGGATATGAG |
| 160 | Nas.D-60 | GGGTGCATCGTTTACGCGTA |
| | | AAAAATCTAAAAAGATATCA |
| | | GCAGGCAAATTTTCCTTCTG |
| | | CTGCTGAGGAAGGATATGAG |
| 161 | Nas.D-61 | GGGTGCATCGTTTACGCGTA |
| | | AAATAAAGAGGATAACTACA |
| | | ATCATCAGCAATCATATCTG |
| | | CTGCTGAGGAAGGATATGAG |
| 162 | Nas.D-62 | GGGTGCATCGTTTACGCGTA |
| | | AATTTAGTAGAAAGGAAAGA |
| | | CGAAGTTTCCTCAGTTTCTG |
| | | CTGCTGAGGAAGGATATGAG |
| 163 | Nas.D-63 | GGGTGCATCGTTTACGCGTA |
| | | AAAATAATAGATCTCAGAAT |
| | | ATGAAAGCAGTTCTTTCCTG |
| | | CTGCTGAGGAAGGATATGAG |
| 164 | Nas.D-64 | GGGTGCATCGTTTACGCGTA |
| | | ACAAGATATTCACAGCAGAT |
| | | TTTAAAAAATTCCTCGTCTG |
| | | CTGCTGAGGAAGGATATGAG |
| 165 | Nas.D-65 | GGGTGCATCGTTTACGCGTA |
| | | AAAAGTTGACAATTAATAAA |
| | | ATCTTCTTAGCATTTTCCTG |
| | | CTGCTGAGGAAGGATATGAG |
| 166 | Nas.D-66 | GGGTGCATCGTTTACGCGTA |
| | | AAACAAAATGAAACTTATAG |
| | | CTCAGCATATTTTGATCCTG |
| | | CTGCTGAGGAAGGATATGAG |
| 167 | Nas.D-67 | GGGTGCATCGTTTACGCGTA |
| | | AATTATCAAAAAAGCAGATT |
| | | TAAGTATACCTCAGTTACTG |
| | | CTGCTGAGGAAGGATATGAG |
| 168 | Nas.D-68 | GGGTGCATCGTTTACGCGTA |
| | | AATAAAATAGCTCAGCAAGG |
| | | AAGTTTTTTTCCTCAAACTG |
| | | CTGCTGAGGAAGGATATGAG |
| 169 | Nas.D-69 | GGGTGCATCGTTTACGCGTA |
| | | AATTTGAGAAAAGAACAGCA |
| | | GACTCAAATCTTTTTAACTG |
| | | CTGCTGAGGAAGGATATGAG |
| 170 | Nas.D-70 | GGGTGCATCGTTTACGCGTA |
| | | ACAGAAAATTAAGCTCAGCA |
| | | ATAGTAATTATCCTAGTCTG |
| | | CTGCTGAGGAAGGATATGAG |
| 171 | Nas.D-71 | GGGTGCATCGTTTACGCGTA |
| | | ATGAAAATAAATCAGTCTCA |
| | | CAGCATTTTAAAACTTCCTG |
| | | CTGCTGAGGAAGGATATGAG |
| 172 | Nas.D-72 | GGGTGCATCGTTTACGCGTA |
| | | TTTACAAGCAACAAAGTTAC |
| | | AATCAGCAGAATTTATCCTG |
| | | CTGCTGAGGAAGGATATGAG |
| 173 | Nas.D-73 | GGGTGCATCGTTTACGCGTA |
| | | AAAAATTGTCTATAGCACTT |
| | | TTAGATTCCCAAACTAACTG |
| | | CTGCTGAGGAAGGATATGAG |
| 174 | Nas.D-74 | GGGTGCATCGTTTACGCGTA |
| | | AAAAAATCAGCAAAATCGAA |
| | | AACTCATGCAGTTTGTCCTG |
| | | CTGCTGAGGAAGGATATGAG |
| 175 | Nas.D-75 | GGGTGCATCGTTTACGCGTA |
| | | AAAAATTCCTTAAAAATTTA |
| | | ACTAACTGGATAGGTCTCTG |
| | | CTGCTGAGGAAGGATATGAG |
| 176 | Nas.D-76 | GGGTGCATCGTTTACGCGTA |
| | | AAACAAAATTTCTGACAGCA |
| | | ATTCCTTCGTTAAAAATCTG |
| | | CTGCTGAGGAAGGATATGAG |
| 177 | Nas.D-77 | GGGTGCATCGTTTACGCGTA |
| | | AATTATTAAAAAAATCAGCA |
| | | AAGTTTATTTCCCACGGCTG |
| | | CTGCTGAGGAAGGATATGAG |
| 178 | Nas.D-78 | GGGTGCATCGTTTACGCGTA |
| | | ATTAATCAAACAATAGCAGC |
| | | AAATCTCAGCAATTTTCCTG |
| | | CTGCTGAGGAAGGATATGAG |
| 179 | Nas.D-79 | GGGTGCATCGTTTACGCGTA |
| | | ATTTGAAAGTCTCATAAATT |
| | | TTTTTTTTTTTTTCAATCTG |
| | | CTGCTGAGGAAGGATATGAG |
| 180 | Nas.D-80 | GGGTGCATCGTTTACGCGTA |
| | | AAAATTCAGCATGATTTCAA |
| | | TTACTCCTTTCATTGATCTG |
| | | CTGCTGAGGAAGGATATGAG |
| 181 | Nas.D-81 | GGGTGCATCGTTTACGCGTA |
| | | AAATAAATAAAAATCAGTAG |
| | | CAATCTTTCTCACAGTGCTG |
| | | CTGCTGAGGAAGGATATGAG |
| 182 | Nas.D-82 | GGGTGCATCGTTTACGCGTA |
| | | AATAAAAAGCAGATCTCAGC |
| | | AAAACTCGTAAATTCAACTG |
| | | CTGCTGAGGAAGGATATGAG |
| 183 | Nas.D-83 | GGGTGCATCGTTTACGCGTA |
| | | AATAATGAAGGACTCAGACA |
| | | GTTAAAAGATGCATTAACTG |
| | | CTGCTGAGGAAGGATATGAG |
| 184 | Nas.D-84 | GGGTGCATCGTTTACGCGTA |
| | | AAAAAGATCAATATGAAAAT |
| | | CAGCAGTTAATATCTTCCTG |
| | | CTGCTGAGGAAGGATATGAG |
| 185 | Nas.D-85 | GGGTGCATCGTTTACGCGTA |
| | | AAAATAACAAACTTCTCAGC |
| | | TGTTTAATATCTCCTGACTG |
| | | CTGCTGAGGAAGGATATGAG |
| 186 | Nas.D-86 | GGGTGCATCGTTTACGCGTA |
| | | AAATTAAACAAATAGCTCAG |
| | | CACGAAAATTTGCGTAACTG |
| | | CTGCTGAGGAAGGATATGAG |
| 187 | Nas.D-87 | GGGTGCATCGTTTACGCGTA |
| | | ATTAAAAAACCTTCACACAG |
| | | AAAACATTCCTCAATTTCTG |
| | | CTGCTGAGGAAGGATATGAG |
| 188 | Nas.D-88 | GGGTGCATCGTTTACGCATT |
| | | TTCGTTTTATTTTAGTTTAA |
| | | TTGCGTTTAGTATCTGGCTG |
| | | CTGCTGAGGAAGGATATGAG |
| 189 | Nas.D-89 | GGGTGCATCGTTTACGCGTA |
| | | AAAAGTATAAAGGTTAGAAA |
| | | TTCAGCAGTTTGATATCCTG |
| | | CTGCTGAGGAAGGATATGAG |
| 190 | Nas.D-90 | GGGTGCATCGTTTACGCGTA |
| | | AAAAGGAGAATTAGTACTCA |
| | | CCAGTCGTTTAAAATTTCTG |
| | | CTGCTGAGGAAGGATATGAG |
| 191 | Nas.D-91 | GGGTGCATCGTTTACGCGTA |
| | | AAAATAAATAACTACGAGAT |
| | | CTCAGCAGATCATTATCCTG |
| | | CTGCTGAGGAAGGATATGAG |
| 192 | Nas.D-92 | GGGTGCATCGTTTACGCGTA |
| | | AAATGGTTTTTCAGCAGTTA |
| | | ACATAATGCCTCAGTTTCTG |
| | | CTGCTGAGGAAGGATATGAG |
| 193 | Nas.D-93 | GGGTGCATCGTTTACGCGTA |
| | | AATAACAAAAATCTCAGCTT |
| | | TTGCAGAATTTATCCACCTG |
| | | CTGCTGAGGAAGGATATGAG |
| 194 | Nas.D-94 | GGGTGCATCGTTTACGCGTA |
| | | AATAAACTCACAGCAGAAAA |
| | | AATTCCTTCAACTTGTACTG |
| | | CTGCTGAGGAAGGATATGAG |
| 195 | Nas.D-95 | GGGTGCATCGTTTACGCAGT |
| | | AGTTAATAACAAATAGTCAG |
| | | CAGTTTTGTCCTTCATTCTG |
| | | CTGCTGAGGAAGGATATGAG |
| 196 | Nas.D-96 | GGGTGCATCGTTTACGCGTA |
| | | AAAATAGCAGTAGATAGCGG |
| | | CAGTTTTGTATTTGTTACTG |
| | | CTGCTGAGGAAGGATATGAG |
| 197 | Nas.D-97 | GGGTGCATCGTTTACGCGTA |
| | | AAAATTTAAATAACTCAGCA |
| | | ATCATAGATCCGACTGACTG |
| | | CTGCTGAGGAAGGATATGAG |
| 198 | Nas.D-98 | GGGTGCATCGTTTACGCGTA |
| | | AAGAACAGCTGACAAGAAAT |
| | | TCAAACCTTCAGATTTTCTG |
| | | CTGCTGAGGAAGGATATGAG |
| 199 | Nas.D-99 | GGGTGCATCGTTTACGCGTA |
| | | AAGATAATAAGCAGTATTCA |
| | | GCAGATTTGTAAGGTTTCTG |
| | | CTGCTGAGGAAGGATATGAG |
| 200 | Nas.D- | GGGTGCATCGTTTACGCGTA |
| | 100 | AATAAGAGGCAGACAGTATT |
| | | ACAAATATCCTAAAATACTG |
| | | CTGCTGAGGAAGGATATGAG |

TABLE 5
List of conserved motifs.

| SEQ ID NO | Sequence |
|---|---|
| 201 | AAACAAAAAGA |
| 202 | UAAAAAUCA |
| 203 | AAACAAAAAGA |
| 204 | TAAAAATCA |
| 205 | AUAAAAAUUUAAA |
| 206 | ATAAAAATTTAAA |
| 207 | GUAAAAAUUAAA |
| 208 | GTAAAAATTAAA |
| 209 | GUAAAAAAA |
| 210 | UNAGCANUUU |
| 211 | GTAAAAAAA |
| 212 | TNAGCANTTT |

TABLE 6
List of protein sequences

| SEQ | | |
|---|---|---|
| ID | | |
| NO | Description | Sequence |
| 213 | ICAM-1 | MAPSSPRPALPALLVLLGAL |
| | | FPGPGNAQTSVSPSKVILPR |
| | | GGSVLVTCSTSCDQPKLLGI |
| | | ETPLPKKELLLPGNNRKVYE |
| | | LSNVQEDSQPMCYSNCPDGQ |
| | | STAKTFLTVYWTPERVELAP |
| | | LPSWQPVGKNLTLRCQVEGG |
| | | APRANLTVVLLRGEKELKRE |
| | | PAVGEPAEVTTTVLVRRDHH |
| | | GANFSCRTELDLRPQGLELF |
| | | ENTSAPYQLQTFVLPATPPQ |
| | | LVSPRVLEVDTQGTVVCSLD |
| | | GLFPVSEAQVHLALGDQRLN |
| | | PTVTYGNDSFSAKASVSVTA |
| | | EDEGTQRLTCAVILGNQSQE |
| | | TLQTVTIYSFPAPNVILTKP |
| | | EVSEGTEVTVKCEAHPRAKV |
| | | TLNGVPAQPLGPRAQLLLKA |
| | | TPEDNGRSFSCSATLEVAGQ |
| | | LIHKNQTRELRVLYGPRLDE |
| | | RDCPGNWTWPENSQQTPMCQ |
| | | AWGNPLPELKCLKDGTFPLP |
| | | IGESVTVTRDLEGTYLCRAR |
| | | STQGEVTRKVTVNVLSPRYE |
| | | IVIITVVAAAVIMGTAGLST |
| | | YLYNRQRKIKKYRLQQAQKG |
| | | TPMKPNTQATPP |
| 214 | Extracellular | QTSVSPSKVILPRGGSVLVT |
| | domain of | CSTSCDQPKLLGIETPLPKK |
| | ICAM-1 | ELLLPGNNRKVYELSNVQED |
| | | SQPMCYSNCPDGQSTAKTFL |
| | | TVYWTPERVELAPLPSWQPV |
| | | GKNLTLRCQVEGGAPRANLT |
| | | VVLLRGEKELKREPAVGEPA |
| | | EVTTTVLVRRDHHGANFSCR |
| | | TELDLRPQGLELFENTSAPY |
| | | QLQTFVLPATPPQLVSPRVL |
| | | EVDTQGTVVCSLDGLFPVSE |
| | | AQVHLALGDQRLNPTVTYGN |
| | | DSFSAKASVSVTAEDEGTQR |
| | | LTCAVILGNQSQETLQTVTI |
| | | YSFPAPNVILTKPEVSEGTE |
| | | VTVKCEAHPRAKVTLNGVPA |
| | | QPLGPRAQLLLKATPEDNGR |
| | | SFSCSATLEVAGQLIHKNQT |
| | | RELRVLYGPRLDERDCPGNW |
| | | TWPENSQQTPMCQAWGNPLP |
| | | ELKCLKDGTFPLPIGESVTV |
| | | TRDLEGTYLCRARSTQGEVT |
| | | RKVTVNVLSPRYE |
| 215 | Ig-like C2- | GGSVLVTCSTSCDQPKLLGI |
| | type 1 | ETPLPKKELLLPGNNRKVYE |
| | domain | LSNVQEDSQPMCYSNCPDGQ |
| | | STA |
| 216 | Ig-like C2- | GKNLTLRCQVEGGAPRANLT |
| | type 2 | VVLLRGEKELKREPAVGEPA |
| | domain | EVTTTVLVRRDHHGANFSCR |
| | | TELDLR |
| 217 | Ig-like C2- | DTQGTVVCSLDGLFPVSEAQ |
| | type 3 | VHLALGDQRLNPTVTYGNDS |
| | domain | FSAKASVSVTAEDEGTQRLT |
| | | CAVILGNQ |
| 218 | Ig-like C2- | GTEVTVKCEAHPRAKVTLNG |
| | type 4 | VPAQPLGPRAQLLLKATPED |
| | domain | NGRSFSCSATLEVA |
| 219 | Ig-like C2- | NSQQTPMCQAWGNPLPELKC |
| | type 5 | LKDGTFPLPIGESVTVTRDL |
| | domain | EGTYLCRARSTQG |
| 220 | Fragment of | QTSVSPSKVILPR |
| | ICAM-1 | |
| 221 | Fragment of | SCDQPKLLGI |
| | ICAM-2 | |
| 222 | Fragment of | PKKELLLPGNNRKVYE |
| | ICAM-3 | |
| 223 | Fragment of | YSNCPDGQSTAKTFL |
| | ICAM-4 | |
| 231 | ICAM-3 | MATMVPSVLWPRACWTLLVC |
| | | CLLTPGVQGQEFLLRVEPQN |
| | | PVLSAGGSLFVNCSTDCPSS |
| | | EKIALETSLSKELVASGMGW |
| | | AAFNLSNVTGNSRILCSVYC |
| | | NGSQITGSSNITVYRLPERV |
| | | ELAPLPPWQPVGQNFTLRCQ |
| | | VEDGSPRTSLTVVLLRWEEE |
| | | LSRQPAVEEPAEVTATVLAS |
| | | RDDHGAPFSCRTELDMQPQG |
| | | LGLFVNTSAPRQLRTFVLPV |
| | | TPPRLVAPRFLEVETSWPVD |
| | | CTLDGLFPASEAQVYLALGD |
| | | QMLNATVMNHGDTLTATATA |
| | | TARADQEGAREIVCNVTLGG |
| | | ERREARENLTVFSFLGPIVN |
| | | LSEPTAHEGSTVTVSCMAGA |
| | | RVQVTLDGVPAAAPGQPAQL |
| | | QLNATESDDGRSFFCSATLE |
| | | VDGEFLHRNSSVQLRVLYGP |
| | | KIDRATCPQHLKWKDKTRHV |
| | | LQCQARGNPYPELRCLKEGS |
| | | SREVPVGIPFFVNVTHNGTY |
| | | QCQASSSRGKYTLVVVMDIE |
| | | AGSSHFVPVFVAVLLTLGVV |
| | | TIVLALMYVFREHQRSGSYH |
| | | VREESTYLPLTSMQPTEAMG |
| | | EEPSRAE |
| 232 | Extracellular | QEFLLRVEPQNPVLSAGGSL |
| | domain of | FVNCSTDCPSSEKIALETSL |
| | ICAM-3 | SKELVASGMGWAAFNLSNVT |
| | | GNSRILCSVYCNGSQITGSS |
| | | NITVYRLPERVELAPLPPWQ |
| | | PVGQNFTLRCQVEDGSPRTS |
| | | LTVVLLRWEEELSRQPAVEE |
| | | PAEVTATVLASRDDHGAPFS |
| | | CRTELDMQPQGLGLFVNTSA |
| | | PRQLRTFVLPVTPPRLVAPR |
| | | FLEVETSWPVDCTLDGLFPA |
| | | SEAQVYLALGDQMLNATVMN |
| | | HGDTLTATATATARADQEGA |
| | | REIVCNVTLGGERREARENL |
| | | TVFSFLGPIVNLSEPTAHEG |
| | | STVTVSCMAGARVQVTLDGV |
| | | PAAAPGQPAQLQLNATESDD |
| | | GRSFFCSATLEVDGEFLHRN |
| | | SSVQLRVLYGPKIDRATCPQ |
| | | HLKWKDKTRHVLQCQARGNP |
| | | YPELRCLKEGSSREVPVGIP |
| | | FFVNVTHNGTYQCQASSSRG |
| | | KYTLVVVMDIEAGSSH |
| 233 | ICAM-5 | MPGPSPGLRRALLGLWAALG |
| | | LGLFGLSAVSQEPFWADLQP |
| | | RVAFVERGGSLWLNCSTNCP |
| | | RPERGGLETSLRRNGTQRGL |
| | | RWLARQLVDIREPETQPVCF |
| | | FRCARRTLQARGLIRFQRPD |
| | | RVELMPLPPWQPVGENFTLS |
| | | CRVPGAGPRASLTLTLLRGA |
| | | QELIRRSFAGEPPRARGAVL |
| | | TATVLARREDHGANFSCRAE |
| | | LDLRPHGLGLFENSSAPREL |
| | | RTFSLSPDAPRLAAPRLLEV |
| | | GSERPVSCTLDGLFPASEAR |
| | | VYLALGDQNLSPDVTLEGDA |
| | | FVATATATASAEQEGARQLV |
| | | CNVTLGGENRETRENVTIYS |
| | | FPAPLLTLSEPSVSEGQMVT |
| | | VTCAAGAQALVTLEGVPAAV |
| | | PGQPAQLQLNATENDDRRSF |
| | | FCDATLDVDGETLIKNRSAE |
| | | LRVLYAPRLDDSDCPRSWTW |
| | | PEGPEQTLRCEARGNPEPSV |
| | | HCARSDGGAVLALGLLGPVT |
| | | RALSGTYRCKAANDQGEAVK |
| | | DVTLTVEYAPALDSVGCPER |
| | | ITWLEGTEASLSCVAHGVPP |
| | | PDVICVRSGELGAVIEGLLR |
| | | VAREHAGTYRCEATNPRGSA |
| | | AKNVAVTVEYGPRFEEPSCP |
| | | SNWTWVEGSGRLFSCEVDGK |
| | | PQPSVKCVGSGGATEGVLLP |
| | | LAPPDPSPRAPRIPRVLAPG |
| | | IYVCNATNRHGSVAKTVVVS |
| | | AESPPEMDESTCPSHQTWLE |
| | | GAEASALACAARGRPSPGVR |
| | | CSREGIPWPEQQRVSREDAG |
| | | TYHCVATNAHGTDSRTVTVG |
| | | VEYRPVVAELAASPPGGVRP |
| | | GGNFTLTCRAEAWPPAQISW |
| | | RAPPGALNIGLSSNNSTLSV |
| | | AGAMGSHGGEYECAATNAHG |
| | | RHARRITVRVAGPWLWVAVG |
| | | GAAGGAALLAAGAGLAFYVQ |
| | | STACKKGEYNVQEAESSGEA |
| | | VCLNGAGGGAGGAAGAEGGP |
| | | EAAGGAAESPAEGEVFAIQL |
| | | TSA |
| 234 | Extracellular | EPFWADLQPRVAFVERGGSL |
| | domain of | WLNCSTNCPRPERGGLETSL |
| | ICAM-5 | RRNGTQRGLRWLARQLVDIR |
| | | EPETQPVCFFRCARRTLQAR |
| | | GLIRTFQRPDRVELMPLPPW |
| | | QPVGENFTLSCRVPGAGPRA |
| | | SLTLTLLRGAQELIRRSFAG |
| | | EPPRARGAVLTATVLARRED |
| | | HGANFSCRAELDLRPHGLGL |
| | | FENSSAPRELRTFSLSPDAP |
| | | RLAAPRLLEVGSERPVSCTL |
| | | DGLFPASEARVYLALGDQNL |
| | | SPDVTLEGDAFVATATATAS |
| | | AEQEGARQLVCNVTLGGENR |
| | | ETRENVTIYSFPAPLLTLSE |
| | | PSVSEGQMVTVTCAAGAQAL |
| | | VTLEGVPAAVPGQPAQLQLN |
| | | ATENDDRRSFFCDATLDVDG |
| | | ETLIKNRSAELRVLYAPRLD |
| | | DSDCPRSWTWPEGPEQTLRC |
| | | EARGNPEPSVHCARSDGGAV |
| | | LALGLLGPVTRALSGTYRCK |
| | | AANDQGEAVKDVTLTVEYAP |
| | | ALDSVGCPERITWLEGTEAS |
| | | LSCVAHGVPPPDVICVRSGE |
| | | LGAVIEGLLRVAREHAGTYR |
| | | CEATNPRGSAAKNVAVTVEY |
| | | GPRFEEPSCPSNWTWVEGSG |
| | | RLFSCEVDGKPQPSVKCVGS |
| | | GGATEGVLLPLAPPDPSPRA |
| | | PRIPRVLAPGIYVCNATNRH |
| | | GSVAKTVVVSAESPPEMDES |
| | | TCPSHQTWLEGAEASALACA |
| | | ARGRPSPGVRCSREGIPWPE |
| | | QQRVSREDAGTYHCVATNAH |
| | | GTDSRTVTVGVEYRPVVAEL |
| | | AASPPGGVRPGGNFTLTCRA |
| | | EAWPPAQISWRAPPGALNIG |
| | | LSSNNSTLSVAGAMGSHGGE |
| | | YECAATNAHGRHARRITVRV |
| | | AGPW |

### Combinations

A. An aptamer composition comprising at least one oligonucleotide consisting of: deoxyribonucleotides, ribonucleotides, derivatives of deoxyribonucleotides, derivatives of ribonucleotides, and mixtures thereof; wherein the aptamer composition has a binding affinity for intercellular adhesion molecule 1 (ICAM-1); and wherein the aptamer is configured to reduce the binding of one or more human rhinoviruses to the intercellular adhesion molecule 1 (ICAM-1) and wherein the aptamer composition comprises
   i. at least one oligonucleotide selected from the group consisting of oligonucleotides with at least 80% nucleotide sequence identity to sequences selected from the group consisting of SEQ ID NO: 1 to SEQ ID NO: 200; and/or.
   ii. at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.
B. The aptamer composition according to Paragraph A, wherein the at least one oligonucleotide is selected from the group consisting of SEQ ID NO: 2, SEQ ID NO: 4, SEQ ID NO: 5, SEQ ID NO: 7, and SEQ ID NO: 8.
C. The aptamer composition according to Paragraph A-B, wherein the at least one oligonucleotide shows at least 90%, or 95%, or 96%, or 97%, or 98% or 99% nucleotide sequence identity to sequences selected from the group consisting of SEQ ID NO: 1 to SEQ ID NO: 200, or wherein the at least one oligonucleotide shows at least 90%, or 95%, or 96%, or 97%, or 98% or 99% nucleotide sequence identity to sequences selected from the group consisting of SEQ ID NO: 2, SEQ ID NO: 4, SEQ ID NO: 5, SEQ ID NO: 7, and SEQ ID NO: 8.
D. The aptamer composition according to Paragraph A-C, comprising at least one oligonucleotide selected from the group consisting of SEQ ID NO: 1 to SEQ ID NO: 200.
E. The aptamer composition according to Paragraph A-D, comprising at least one oligonucleotide selected from the group consisting of SEQ ID NO: 2, SEQ ID NO: 4, SEQ ID NO: 5, SEQ ID NO: 7, and SEQ ID NO: 8.
F. The aptamer composition according to Paragraph A-E, wherein the at least one oligonucleotide comprises natural or non-natural nucleobases; preferably wherein the non-natural nucleobases are selected from the group comprising hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, and mixtures thereof.
G. The aptamer composition according to Paragraph A-F, wherein the nucleosides of the at least one oligonucleotide are linked by a chemical motif selected from the group comprising natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, and mixtures thereof.
H. The aptamer composition according to Paragraph A-G, where the derivatives of ribonucleotides or the derivatives of deoxyribonucleotides are selected from the group comprising locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.
I. The aptamer composition according to Paragraph A-H, further comprising at least one polymeric material, wherein the at least one polymeric material is covalently linked to the at least one oligonucleotide; preferably wherein the at least one polymeric material is polyethylene glycol.
J. The aptamer composition according to Paragraph A-I wherein the nucleotides at the 5′- and 3′-ends of the at least one oligonucleotide are inverted.
K. The aptamer composition according to Paragraph A-J, wherein at least one nucleotide of the at least one oligonucleotide is fluorinated at the 2′ position of the pentose group; preferably wherein the pyrimidine nucleotides of the at least one oligonucleotide are fluorinated at the 2′ position of the pentose group.
L. The aptamer composition according to Paragraph A-K, wherein the at least one oligonucleotide is covalently or non-covalently attached to one or more active ingredients, wherein the one or more active ingredients are selected from the group consisting of: respiratory illness treatment agents, cold-treatment agents, flu-treatment agents, antiviral agents, antimicrobial agents, cooling agents, malodor absorbing agents, natural extracts, peptides, enzymes, pharmaceutical active ingredients, metal compounds, and combinations thereof.
M. An aptamer composition comprising at least one peptide or protein, wherein the peptide or protein is translated from at least one of the oligonucleotides of anyone of paragraphs A-L.
N. The aptamer composition according to Paragraph A-M wherein the aptamer has a binding affinity for the Ig-like C2-type 1 domain (SEQ ID NO: 215) of the intercellular adhesion molecule 1 (ICAM-1), any post-translationally modified versions of said domain, and mixtures thereof.
O. The aptamer composition according to Paragraph A-M, wherein the at least one oligonucleotide is covalently or non-covalently attached to one or more nanomaterials comprising one or more active ingredients.
P. A personal health care composition comprising the at least one aptamer composition according to paragraph A-O.
Q. The personal health care composition according to paragraph P, wherein the at least one nucleic acid aptamer is covalently or non-covalently attached to one or more active ingredients, wherein said one or more active ingredients are selected from the group comprising: respiratory illness treatment agents, cold-treatment agents, flu-treatment agents, antiviral agents, antimicrobial agents, cooling agents, malodor absorbing agents, natural extracts, peptides, enzymes, pharmaceutical active ingredients, metal compounds, and mixtures thereof.
R. The aptamer composition according to paragraph A-O or the personal health care composition according to paragraph P or Q for inhibiting human rhinovirus infection by inhibiting binding to the intercellular adhesion molecule 1 (ICAM-1) and thereby inhibiting entering into cells within the nasal cavity and throat and/or for preventing and treating symptoms associated with respiratory tract viral infections, preferably by delivering the composition to the upper respiratory tract.
S. A method for delivering a personal health care composition to the upper respiratory tract comprising administering to a subject in need thereof a personal health care composition comprising at least one nucleic acid aptamer, wherein the aptamer has a binding affinity for intercellular adhesion molecule 1 (ICAM-1) and wherein the aptamer is configured to reduce the binding of one or more human rhinoviruses to the intercellular adhesion molecule 1 (ICAM-1).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Values disclosed herein as ends of ranges are not to be understood as being strictly limited to the exact numerical values recited. Instead, unless otherwise specified, each numerical range is intended to mean both the recited values and any real numbers including integers within the range. For example, a range disclosed as “1 to 10” is intended to mean “1, 2, 3, 4, 5, 6, 7, 8, 9, and 10” and a range disclosed as “1 to 2” is intended to mean “1.1, 1.2, 1.3, 1.4, 1.5, 1.6, 1.7, 1.8, 1.9, and 2.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in

1. Aptamer composition comprising at least one oligonucleotide consisting of: deoxyribonucleotides, ribonucleotides, derivatives of deoxyribonucleotides, derivatives of ribonucleotides, and mixtures thereof, wherein the aptamer composition has a binding affinity for intercellular adhesion molecule 1 (ICAM-1) and wherein the aptamer is configured to reduce the binding of one or more human rhinoviruses to the intercellular adhesion molecule 1 (ICAM-1) and wherein the aptamer composition comprises
at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

2. The aptamer composition of claim 1, wherein the at least one oligonucleotide shows at least 95%, nucleotide sequence identity to sequences selected from the group consisting of SEQ ID NO: 1 to SEQ ID NO: 200.

3. The aptamer composition of claim 1, comprising at least one oligonucleotide selected from the group consisting of SEQ ID NO: 1 to SEQ ID NO: 200.

4. The aptamer composition of claim 3, comprising at least one oligonucleotide selected from the group consisting of SEQ ID NO: 2, SEQ ID NO: 4, SEQ ID NO: 5, SEQ ID NO: 7, and SEQ ID NO: 8.

5. The aptamer composition of claim 1, wherein the at least one oligonucleotide comprises natural or non-natural nucleobases.

6. The aptamer composition of claim 5, wherein the non-natural nucleobases are selected from the group comprising hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, and mixtures thereof.

7. The aptamer composition of claim 1, wherein the nucleosides of the at least one oligonucleotide are linked by a chemical motif selected from the group comprising natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, and mixtures thereof.

8. The aptamer composition of claim 1, where the derivatives of ribonucleotides or the derivatives of deoxyribonucleotides are selected from the group comprising locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.

9. The aptamer composition of claim 1, further comprising at least one polymeric material, wherein the at least one polymeric material is covalently linked to the at least one oligonucleotide.

10. The aptamer composition of claim 9, wherein the at least one polymeric material is polyethylene glycol.

11. The aptamer composition of claim 1, wherein the nucleotides at the 5′- and 3′-ends of the at least one oligonucleotide are inverted.

12. The aptamer composition of claim 1, wherein at least one nucleotide of the at least one oligonucleotide is fluorinated at the 2′ position of the pentose group.

13. The aptamer composition of claim 1, wherein the pyrimidine nucleotides of the at least one oligonucleotide are fluorinated at the 2′ position of the pentose group.

14. The aptamer composition of claim 1, wherein the at least one oligonucleotide is covalently or non-covalently attached to one or more active ingredients, wherein the one or more active ingredients are selected from the group consisting of: respiratory illness treatment agents, cold-treatment agents, flu-treatment agents, antiviral agents, antimicrobial agents, cooling agents, malodor absorbing agents, natural extracts, peptides, enzymes, pharmaceutical active ingredients, metal compounds, and combinations thereof.

15. Aptamer composition comprising at least one oligonucleotide consisting of: deoxyribonucleotides, ribonucleotides, derivatives of deoxyribonucleotides, derivatives of ribonucleotides, and mixtures thereof; wherein the aptamer has a binding affinity for the Ig-like C2-type 1 domain (SEQ ID NO: 215) of the intercellular adhesion molecule 1 (ICAM-1), any post-translationally modified versions of said domain, and mixtures thereof, wherein the aptamer composition comprises
at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

16. The aptamer composition of claim 15, comprising at least one oligonucleotide selected from the group consisting of oligonucleotides selected from the group consisting of SEQ ID NO: 1 to SEQ ID NO: 200.

17. The aptamer composition of claim 15, comprising at least one oligonucleotide selected from the group consisting of SEQ ID NO: 2, SEQ ID NO: 4, SEQ ID NO: 5, SEQ ID NO: 7, and SEQ ID NO: 8.

18. Aptamer composition comprising at least one peptide or protein, wherein the peptide or protein is translated from at least one of the oligonucleotides of claim 1.

19. Personal health care composition comprising at least one nucleic acid aptamer; wherein the nucleic acid aptamer has a binding affinity for intercellular adhesion molecule 1 (ICAM-1), and wherein the aptamer is configured to reduce the binding of one or more human rhinoviruses to the intercellular adhesion molecule 1 (ICAM-1) wherein the aptamer composition comprises
at least one oligonucleotide comprising one or more motifs selected from the group consisting of SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, and SEQ ID NO: 212.

20. The personal health care composition of claim 19, comprising at least one oligonucleotide selected from the group consisting of oligonucleotides selected from the group consisting of SEQ ID NO: 1 to SEQ ID NO: 200.

21. The personal health care composition of claim 20, comprising at least one oligonucleotide selected from the group consisting of SEQ ID NO: 2, SEQ ID NO: 4, SEQ ID NO: 5, SEQ ID NO: 7, and SEQ ID NO: 8.

22. The personal health care composition of claim 19, wherein the at least one nucleic acid aptamer is covalently or non-covalently attached to one or more active ingredients, wherein said one or more active ingredients are selected from the group comprising: respiratory illness treatment agents, cold-treatment agents, flu-treatment agents, antiviral agents, antimicrobial agents, cooling agents, malodor absorbing agents, natural extracts, peptides, enzymes, pharmaceutical active ingredients, metal compounds, and mixtures thereof.

23. Method for delivering a personal health care composition to the upper respiratory tract comprising administering to a subject in need thereof the personal health care composition of claim 19.

24. The aptamer composition of claim 15, wherein the at least one oligonucleotide shows at least 95%, nucleotide sequence identity to sequences selected from the group consisting of SEQ ID NO: 1 to SEQ ID NO: 200.

25. The personal health care composition of claim 19, wherein the at least one oligonucleotide shows at least 95%, nucleotide sequence identity to sequences selected from the group consisting of SEQ ID NO: 1 to SEQ ID NO: 200.
